(19) United States
(12) Patent Application Publication
(10) Pub. No.: US 2023/0405224 A1
(43) Pub. Date: Dec. 21, 2023

(54) AUTONOMOUS DOSE DETERMINATION FOR PEN DELIVERY OF MEDICAMENT

(71) Applicant: Beta Bionics, Inc., Concord, MA (US)

(72) Inventors: Michael J. Rosinko, Las Vegas, NV (US); Himanshu Patel, Rancho Santa Margarita, CA (US); Firas H. El-Khatib, Allston, MA (US)

(21) Appl. No.: 18/296,443

(22) Filed: Apr. 6, 2023

Related U.S. Application Data
(60) Provisional application No. 63/362,868, filed on Apr. 12, 2022.

(51) Int. Cl. A61M 5/172; G16H 20/17

(52) U.S. Cl. CPC A61M 5/1723; G16H 20/17; A61M 2230/201; A61M 2205/502

(57) ABSTRACT

A glucose level control system may be configured to determine a recommended medicament dose based at least in part on a subject's therapy data and/or user-specified medicament related inputs. The therapy data may include glucose level data and medicament delivery data. The user-specified medicament related inputs may include qualitative meal announcements. For example, the user can specify that the subject consumed a small, medium, or large meal. The glucose level control system may be configured to output an indication of the recommended medicament dose to a display. The glucose level control system may be configured to output a dose control signal to preconfigure a smart pen to deliver the recommended medicament dose. The glucose level control system may be configured to generate an alert if it determines, based at least in part on glucose level data and/or medicament delivery data associated with the subject, that trigger criteria have been satisfied.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

### TECHNICAL FIELD

The present disclosure relates to ambulatory medical devices, such as glucose level control systems, that provide therapy to a subject.

### BACKGROUND

Sustained delivery, pump driven medicament injection devices generally include a delivery cannula mounted in a subcutaneous manner through the skin of the patient at an infusion site. The pump draws medicine from a reservoir and delivers it to the patient via the cannula. The injection device typically includes a channel that transmits a medicament from an inlet port to the delivery cannula which results in delivery to the subcutaneous tissue layer where the delivery cannula terminates. Some infusion devices are configured to deliver one medicament to a patient while others are configured to deliver multiple medicaments to a patient.

### SUMMARY

The systems, methods, and devices of this disclosure each have several innovative aspects, no single one of which is solely responsible for all the desirable attributes disclosed herein. Details of one or more implementations of the subject matter described in this specification are set forth in the accompanying drawings and the description below.

Certain embodiments of the present disclosure relate to a glucose level control system configured to determine a long-acting insulin dose recommendation based on administration of fast-acting insulin during a first therapy period, and to generate a dose control signal that causes glucose control therapy using the fast-acting insulin to be provided to a subject during a second therapy period after a long-acting insulin dose is administered to the subject based on the long-acting insulin dose recommendation. The glucose level control system may include: a medicament delivery interface configured to operatively connect to an ambulatory medicament pump for infusing medicament into the subject; a memory configured to store specific computer-executable instructions; and a hardware processor in communication with the memory and configured to execute the specific computer-executable instructions. The computer-executable instructions may be configured to at least: cause first glucose control therapy to be delivered to the subject during the first therapy period, wherein the first glucose control therapy comprises dosing of the fast-acting insulin, wherein the first therapy period is at least a day; determine the long-acting insulin dose recommendation based at least in part on the dosing of the fast-acting insulin; output an indication of the long-acting insulin dose recommendation; and determine second glucose control therapy to be delivered to the subject during the second therapy period after the long-acting insulin dose is administered to the subject based on the long-acting insulin dose recommendation.

The system of the preceding paragraph can include any combination or sub-combination of the following features: where the hardware processor is further configured to use a control algorithm to generate the dose control signal that causes the glucose control therapy; where the hardware processor is further configured to autonomously cause the first glucose therapy to be delivered to the subject using a control algorithm to autonomously generate dose control signals that cause glucose control therapy to be provided to the subject; where the hardware processor is further configured to output the indication of the long-acting insulin dose recommendation by at least causing the indication of the long-acting insulin dose recommendation to be transmitted to an insulin pen; where the hardware processor is further configured to output the indication of the long-acting insulin dose recommendation by at least causing the indication of the long-acting insulin dose recommendation to be transmitted to an electronic device separate from the glucose level control system; where the long-acting insulin dose recommendation is zero units of long-acting insulin; where the long-acting insulin dose recommendation is determined as a percentage of the total fast-acting insulin administered during the first therapy period; where the hardware processor is further configured to determine the second glucose control therapy to be delivered during the second therapy period comprises zero units of fast-acting insulin; where the hardware processor is further configured to autonomously cause the second glucose therapy to be delivered to the subject using a control algorithm to autonomously generate dose control signals that cause glucose control therapy to be provided to the subject; where the hardware processor is further configured to determine the second glucose control therapy by adjusting dosing of the fast-acting insulin; where said adjusting the dosing of the fast-acting insulin accounts, at least in part, for the long-acting insulin dose administered to the subject; where the dosing of the fast-acting insulin comprises basal dosing of the fast-acting insulin and wherein said adjusting the dosing of the fast-acting insulin comprises adjusting the basal dosing of the fast-acting insulin; where said adjusting the dosing of the basal dosing of the fast-acting insulin comprising adjusting the basal dosing of the fast-acting insulin based at least in part on the long-acting insulin dose; where the basal dosing is based on a basal rate, and wherein said adjusting the basal dosing of the fast-acting insulin comprises adjusting the basal rate; where the basal rate is one of a plurality of basal rates applied during a day, and wherein adjusting the basal rate comprises adjusting at least one of the plurality of basal rates applied during the day; where said adjusting dosing of the fast-acting insulin comprises adjusting correction insulin dosing; where the long-acting insulin dose recommendation is determined based on cumulative dosing of the fast-acting insulin during the first therapy period; where the long-acting insulin dose recommendation is determined based on a fraction of the dosing of the fast-acting insulin during the first therapy period; where the long-acting insulin dose recommendation is determined based on basal dosing of the fast-acting insulin during the first therapy period; where the long-acting insulin dose recommendation is determined based on a total daily dose or a function of a total daily dose of fast-acting insulin administered during the first therapy period; where the long-acting insulin dose recommendation is determined based on a portion of the first therapy period; where the portion of the first therapy period may be associated with a lowest basal rate; where long-acting insulin has a duration of action of at least an absorption time period, and wherein the fast-acting insulin has a duration of action that is less than the absorption time period; where the absorption time period is 12 hours or more; where a length of the second therapy period is based on a duration of action of the long-acting insulin dose; where the first glucose control therapy further comprises dosing of one or more additional medicaments; where the one or more additional medicaments include at least a counter-regulatory agent; where the hardware processor is further configured to modify the long-acting insulin dose recommendation over time based at least in part on glucose level data; where the glucose level data is received from a glucose level sensor operatively connected to the subject; where the hardware processor is further configured to adjust glucose control therapy over time as the long-acting insulin dose recommendation is modified; where adjusting the glucose control therapy over time comprises adjusting a basal rate; where the hardware processor is further configured to receive an indication that the long-acting insulin dose has been or is being administered to the subject within a threshold period of time; where the indication is received in response to a user interaction with a user interface; where the indication is received via a wireless connection with an electronic device; where the wireless connection is a direct connection with the electronic device or a connection over a network; and where the hardware processor is further configured to receive an indication of a prior long-acting insulin dose of a first size received during the first therapy period, and wherein the long-acting insulin dose recommendation comprises a recommended adjustment of the long-acting insulin dose from the first size to a second size.

Certain embodiments of the present disclosure relate to a glucose level control system configured to operate using a selected operating mode selected from one of at least a first glycemic dysregulation control (“GDC1”) mode or a second glycemic dysregulation control (“GDC2”) mode, wherein the selected operating mode is associated with at least one control parameter used by a control algorithm to generate a dose control signal that causes glucose control therapy to be provided to a subject. The glucose level control system may include: a medicament delivery interface configured to operatively connect to a medicament pump for infusing medicament into the subject; a memory configured to store specific computer-executable instructions; and a hardware processor in communication with the memory and configured to execute the specific computer-executable instructions. The computer-executable instructions may be configured to at least: receive an indication of the selected operating mode, wherein the selected operating mode is one of the GDC1 mode or the GDC2 mode; configure the at least one control parameter of the control algorithm based at least in part on the selected operating mode. The at least one control parameter may comprise: a target setpoint parameter; a dosing aggressiveness parameter; a glucose level prediction parameter; a subject model parameter; an insulin pharmacokinetic parameter; or a dosing setting parameter. Further, the computer-executable instructions may be configured to cause the glucose control therapy to be delivered to the subject, wherein the glucose control therapy is delivered based at least in part on a value of the at least one control parameter of the control algorithm used to generate the dose control signal.

The system of the preceding paragraph can include any combination or sub-combination of the following features: where the first glycemic dysregulation control mode is a type one diabetes (“T1D”) mode and the second glycemic dysregulation control mode is a type two diabetes (“T2D”) mode; where the target setpoint parameter comprises a glucose level target or an insulin level target; where the dosing aggressiveness parameter differs based at least in part on the type of insulin; where the dosing aggressiveness parameter differs based at least in part on whether long-acting insulin, fast-acting insulin, or both long-acting insulin and fast-acting insulin is delivered as part of the glucose control therapy; where the indication of the selected operating mode is received in response to a user interaction with a user interface; and where the hardware processor is further configured to execute the specific computer-executable instructions to at least: generate an operating mode selection user interface; cause the operating mode selection user interface to be output for display to a user; and receive the indication of the selected operating mode in response to a user interaction with the operating mode selection user interface. Further, the system of the preceding paragraph can include any combination or sub-combination of the foregoing features and/or the following features: where the indication of the selected operating mode is received via a wireless network connection of an electronic device that hosts an operating mode selection user interface; where the hardware processor is further configured to configure the at least one control parameter by at least setting or adjusting a value of the control parameter; where the hardware processor is further configured to configure the target setpoint parameter by at least setting an upper limit on a target setpoint range; where the upper limit of the target setpoint range is set lower for the second glycemic dysregulation control mode than for the first glycemic dysregulation control mode; where the dosing aggressiveness parameter causes more aggressive glucose control therapy when the selected operating mode corresponds to the second glycemic dysregulation control mode than when the selected operating mode corresponds to the first glycemic dysregulation control mode; where more aggressive glucose control therapy comprises providing larger medicament doses in response to a glucose level excursion; where causing the glucose control therapy to be delivered to the subject comprises autonomously causing the glucose control therapy to be delivered to the subject; where the subject model parameter comprises a weight of the subject; where the insulin pharmacokinetic parameter comprises Tₘₐₓ or T_{1/2max}; where the insulin pharmacokinetic parameter comprises an insulin absorption rate for the subject; and where the dosing setting parameter comprises a food intake size or a carbohydrate ratio.

Certain embodiments of the present disclosure relate to a glucose level control system configured to adapt glucose level control therapy delivered to a subject based at least on a dose of long-acting insulin provided to the subject. The glucose level control system may include: a medicament delivery interface configured to operatively connect to a medicament pump for infusing medicament into the subject; a memory configured to store specific computer-executable instructions; and a hardware processor in communication with the memory and configured to execute the specific computer-executable instructions. The computer-executable instructions may be configured to at least: determine the dose of long-acting insulin provided to the subject; and modify glucose control therapy delivered to the subject during a therapy period based at least in part on the dose of long-acting insulin, wherein the glucose control therapy comprises dosing of fast-acting insulin delivered based at least in part on a control algorithm configured to autonomously generate dose control signals that cause the glucose control therapy to be provided to the subject.

The system of the preceding paragraph can include any combination or sub-combination of the following features: where the dose of long-acting insulin is determined based at least in part on a user input signal received in response to a user interacting with a user interface; where the dose of long-acting insulin is determined based at least in part on a user input signal received from an electronic device in communication with the glucose level control system; where the dose of long-acting insulin is determined based at least in part on a glucose level of the subject over a time period on the order of minutes to hours; where the glucose level of the subject is determined based at least in part on glucose sensor data obtained from a glucose sensor operatively coupled to the subject; where the dose of long-acting insulin is determined based at least in part on a measure of fast-acting insulin administered to the subject over the time period; where modifying the glucose control therapy comprises adapting the control algorithm based at least in part on the dose of long-acting insulin; and where the hardware processor is further configured to modify the glucose control therapy by at least: receiving a glucose level signal from a glucose level sensor operatively connected to the subject; and determining basal dosing of fast-acting insulin based at least in part on the glucose level signal, wherein the glucose level signal reflects a modification to a glucose level of the subject due in part to the dose of the long-acting insulin. Further, the system of the preceding paragraph can include any combination or sub-combination of the foregoing features and the following features: where the hardware processor is further configured to modify the glucose control therapy by at least: determining a basal dosing rate of fast-acting insulin for the subject; and reducing the basal dosing rate by a reduction factor, wherein the reduction factor is based at least in part on the dose of long-acting insulin; where the reduction factor comprises a percentage or a ratio; where the hardware processor is further configured to modify the glucose control therapy by at least providing an indication of the dose of long-acting insulin to an insulin dosing controller configured to use the control algorithm to generate an insulin dose control signal based at least in part on glucose level data and the indication of the dose of long-acting insulin; where the hardware processor is further configured to modify the glucose control therapy by at least reducing one or more fast-acting insulin doses based at least in part on a reduction factor, wherein the reduction factor is determined based at least in part on the dose of long-acting insulin; the hardware processor is further configured to determine a long-acting insulin dose recommendation based at least in part on the glucose control therapy delivered to the subject during the therapy period; where the long-acting dose recommendation comprises a recommended modification to the dose of long-acting insulin; and where the hardware processor is further configured to output an indication of the long-acting insulin dose recommendation.

Certain embodiments of the present disclosure relate to a glucose level control system configured to detect whether a dose of long-acting insulin has been administered to a subject. The glucose level control system may include: a medicament delivery interface configured to operatively connect to a medicament pump for infusing medicament into the subject; a memory configured to store specific computer-executable instructions; and a hardware processor in communication with the memory and configured to execute the specific computer-executable instructions. The computer-executable instructions may be configured to at least: autonomously cause first glucose control therapy to be delivered to the subject during a first therapy period, wherein the first glucose control therapy comprises dosing of fast-acting insulin delivered based at least in part on a control algorithm configured to autonomously generate dose control signals that cause glucose control therapy to be provided to the subject; receive a glucose level signal from a glucose level sensor operatively connected to the subject, wherein the glucose level signal corresponds to the first glucose control therapy; and determine based at least in part on the glucose level signal and the first glucose control therapy whether the dose of long-acting insulin was administered during the first therapy period.

The system of the preceding paragraph can include any combination or sub-combination of the following features: where the hardware processor is further configured to: generate an alert in response to determining that the dose of long-acting insulin was not administered during the first therapy period; and cause the alert to be output to a user; where causing the alert to be output to the user comprises outputting the alert on a display of the glucose level control system; where causing the alert to be output to the user comprises transmitting the alert to an electronic device in communication with the glucose level control system; where the alert comprises a reminder to administer the dose of long-acting insulin; where the hardware processor is further configured to modify second glucose control therapy to be delivered to the subject during a second therapy period in response to determining that the dose of long-acting insulin was not administered during the first therapy period; where modifying the second glucose control therapy comprises adapting the control algorithm based at least in part on the determination of an absence of the dose of long-acting insulin; and where modifying the second glucose control therapy comprises: determining a basal dosing rate of fast-acting insulin for the subject; and incrementing the basal dosing rate by an augmentation factor, wherein the augmentation factor is based at least in part on an absence of the dose of long-acting insulin. Further, the system of the preceding paragraph can include any combination or sub-combination of the foregoing features and the following features: where the augmentation factor comprises a percentage or a ratio; where modifying the second glucose control therapy comprises at least augmenting one or more fast-acting insulin doses based at least in part on an augmentation factor, wherein the augmentation factor is determined based at least in part on the dose of long-acting insulin; where the hardware processor is further configured to determine whether the dose of long-acting insulin was administered during the first therapy period by at least detecting an above-threshold increase in the dosing of fast-acting insulin during at least a portion of the first therapy period; where the hardware processor is further configured to determine whether the dose of long-acting insulin was administered during the first therapy period by at least comparing basal insulin delivery during the first therapy period to basal insulin delivery during an earlier therapy period; where the hardware processor is further configured to determine whether the dose of long-acting insulin was administered during the first therapy period by at least comparing basal insulin delivery during the first therapy period to a baseline basal profile for the subject; where the baseline basal profile is generated based on glucose therapy data associated with prior glucose control therapy of the subject; where the baseline basal profile is generated based on glucose therapy data of one or more users that share one or more physiological characteristics with the subject; where the hardware processor is further configured to determine based at least in part on the glucose level signal and the first glucose control therapy whether a physiological condition of the subject has changed; and where the hardware processor is further configured to: generate an alert in response to determining that the physiological condition of the subject has changed; and cause the alert to be output to a user. Further, the system of the preceding paragraph can include any combination or sub-combination of the foregoing features and the following features: where the alert comprises an indication of the physiological condition that has changed; where the hardware processor is further configured to modify second glucose control therapy to be delivered to the subject during a second therapy period in response to determining that the physiological condition of the subject has changed; where modifying the second glucose control therapy comprises modifying a control parameter of the control algorithm based at least in part on the physiological condition of the subject; where the hardware processor is further configured to determine whether a physiological condition of the subject has changed based at least in part on a glycemic response of the subject to the first glucose control therapy; where responsive to determining that the dose of long-acting insulin was administered, the hardware processor is further configured to determine whether the dose of long-acting insulin exceeds a long-acting insulin dose recommendation; where determining whether the dose of long-acting insulin exceeds a long-acting insulin dose recommendation comprises determining whether the dose of long-acting insulin exceeds the long-acting insulin dose recommendation by a threshold amount; where determining whether the dose of long-acting insulin was administered during the first therapy period comprises determining a probability that the dose of long-acting insulin was administered; where determining whether the dose of long-acting insulin was administered during the first therapy period comprises determining whether a basal rate during the first glucose control therapy corresponds to a threshold degree with an expected ratio of long-acting insulin to fast-acting insulin; where the hardware processor is further configured to account for a counter-regulatory agent dose in determining whether the dose of long-acting insulin was administered during the first therapy period; and where the hardware processor is further configured to account for the counter-regulatory agent dose by modifying an expected fast-acting insulin dosing regimen or an expected long-acting insulin to fast-acting insulin ratio.

### BRIEF DESCRIPTION OF THE DRAWINGS

Throughout the drawings, reference numbers are re-used to indicate correspondence between referenced elements. The drawings are provided to illustrate certain aspects of the subject matter described herein and not to limit the scope thereof.

FIG. 1A illustrates an example blood glucose control system that provides blood glucose control via an ambulatory medicament pump.

FIG. 1B illustrates another example blood glucose control system that provides blood glucose control via an ambulatory medicament pump.

FIG. 1C illustrates a further example blood glucose control system that provides blood glucose control via an ambulatory medicament pump.

FIG. 2A shows a block diagram of an example blood glucose control system.

FIG. 2B shows a block diagram of another example blood glucose control system.

FIG. 2C shows a block diagram of another example blood glucose control system.

FIG. 2D shows a block diagram of another example blood glucose control system.

FIG. 3 is a schematic of an example glucose control system that includes an electronic communications interface.

FIG. 4A shows a block diagram of an example blood glucose control system in online operation mode.

FIG. 4B shows a block diagram of an example blood glucose control system in offline operation mode.

FIG. 5 illustrates a block diagram of a glucose control system in accordance with certain embodiments.

FIG. 6 illustrates a block diagram of a controller system in accordance with certain embodiments.

FIG. 7 presents a flowchart of an example long-acting insulin dose recommendation process in accordance with certain embodiments.

FIG. 8 presents a flowchart of an example operating mode selection process in accordance with certain embodiments.

FIG. 9 presents a flowchart of an example glucose control therapy modification process in accordance with certain embodiments.

FIG. 10 presents a flowchart of an example long-acting insulin dosing determination process in accordance with certain embodiments.

FIG. 11 illustrates an example long-acting insulin recommendation user interface in accordance with certain embodiments.

FIG. 12 illustrates an example operating mode selection user interface in accordance with certain embodiments.

FIG. 13 illustrates an example glucose level control therapy modification user interface in accordance with certain embodiments.

FIG. 14 illustrates an example long-acting insulin administration reminder user interface in accordance with certain embodiments.

FIG. 15A illustrates simulations of the glucose level of a subject in accordance with certain embodiments.

FIG. 15B illustrates simulations of the glucose level of a subject in accordance with certain embodiments.

FIG. 15C illustrates simulations of the glucose level of a subject in accordance with certain embodiments.

FIG. 16 illustrates a block diagram of a glucose level control system in accordance with certain embodiments.

FIG. 17 illustrates an example glucose control system environment that provides blood glucose control via a smart pen in accordance with certain embodiments.

FIG. 18 presents a flowchart of an example medicament dose recommendation process in accordance with certain embodiments.

FIG. 19 presents a flowchart of an example recommendation process for a non-automated medicament dose in accordance with certain embodiments.

FIG. 20 presents a flowchart of an example medicament dose reminder process in accordance with certain embodiments.

FIG. 21 presents a flowchart of an example meal-based medicament dose recommendation process in accordance with certain embodiments.

FIG. 22 illustrates an example user interface for a recommendation process for a non-automated medicament dose in accordance with certain embodiments.

### DETAILED DESCRIPTION

Some embodiments described herein pertain to medicament infusion systems for one or more medicaments and the components of such systems (e.g., infusion pumps, medicament cartridges, cartridge connectors, lumen assemblies, infusion connectors, infusion sets, etc.). Some embodiments pertain to methods of manufacturing infusion systems and components thereof. Some embodiments pertain to methods of using any of the foregoing systems or components for infusing one or more medicaments (e.g., pharmaceutical, hormone, etc.) to a patient. As an exemplary illustration, an infusion system may include an infusion pump, which can include one or more medicament cartridges or can have an integrated reservoir of medicament. An infusion system may include medicament cartridges and cartridge connectors, but not a pump. An infusion system may include cartridge connectors and an infusion pump, but not medicament cartridges. An infusion system may include infusion connectors, a lumen assembly, cartridge connectors, an infusion pump, but not medicament cartridges or an infusion set. A blood glucose control system can operate in conjunction with an infusion system to infuse one or more medicaments, including at least one blood glucose control agent, into a subject. Any feature, structure, component, material, step, or method that is described and/or illustrated in any embodiment in this specification can be used with or instead of any feature, structure, component, material, step, or method that is described and/or illustrated in any other embodiment in this specification. Additionally, any feature, structure, component, material, step, or method that is described and/or illustrated in one embodiment may be absent from another embodiment.

Further, certain embodiments disclosed herein relate to an ambulatory medicament device, such as a glucose level control system, capable of recommending long-acting insulin (“LAI”) (e.g., insulin glargine, insulin detemir, insulin degludec) doses that may be provided to a subject based at least in part on fast-acting insulin (“FAI”) (e.g., insulin lispro, insulin aspart, insulin glulisine) therapy provided to the subject. Long-acting insulin may include insulin associated with a longer absorption time period and/or a longer duration of action (or time of activity) than fast-acting insulin (e.g., long-acting insulin may have a duration of action of 12 hours, 24 hours, 3 days, or more while fast-acting insulin may have a duration of action of 2-3 hours, 4 hours, 6 hours, or less than 12 hours). Additionally, some embodiments disclosed herein relate to a glucose level control system capable of determining whether long-acting insulin has been provided to a subject. Further, the system may modify the fast-acting insulin therapy provided to the subject in response to determining that long-acting insulin has or has not been administered to the subject.

In some cases, the amount of fast-acting insulin administered to a subject may be relatively high within a short period of time, requiring frequent changing of medicament cartridges at high financial cost. For example, some subjects with type 2 diabetes (“T2D”) may have relatively low insulin sensitivity. Consequently, some subjects with T2D may require larger insulin doses or more frequent insulin doses than some subjects with type 1 diabetes (“T1D”). Therefore, some subjects with T2D may need to more frequently change FAI medicament cartridges than some T1D subjects, resulting in higher costs.

Advantageously, the glucose level control system described herein allows a subject to combine fast-acting and long-acting insulin therapy for ease of use and reduction of cost. For example, providing a recommendation of long-acting insulin to the subject allows the subject to offset a portion of the subject's need for fast-acting insulin with long-acting insulin, offsetting a portion of the subject's FAI use with a dose of LAI can reduce the usage rate of FAI and reduce the need for frequent changing of medicament cartridges compared to therapy that relies exclusively on FAI.

In some non-limiting examples, the glucose level control system may be configured to administer both long-acting insulin and fast-acting insulin. In other examples, the glucose level control system may administer only fast-acting insulin. In some such cases, the glucose level control system may adjust the administering of fast-acting insulin based on a determination of administered long-acting insulin. Additionally, or alternatively, the glucose level control system may generate a long-acting insulin dose recommendation. The recommended long-acting insulin dose may be administered by a user via injection therapy or, if supported, by the glucose level control system. In some examples, the glucose level control system may receive glucose level data from a glucose level sensor capable of detecting the subject's glucose levels, from which the glucose level control system may determine that long-acting insulin was provided to the subject. In other examples, the user may enter the long-acting insulin dose into the glucose level control system manually (e.g., via a user interface).

For instance, the glucose level control system may provide fast-acting insulin therapy to the subject. The system may recommend, based in part on therapy data associated with the administered fast-acting insulin, that a particular dose of long-acting insulin be administered to the subject. The glucose level control system may determine that the subject has been provided the dose of long-acting insulin via a sensor or user input. The glucose level control system may modify later fast-acting insulin doses provided to the subject based in part on the determination that the subject has received a long-acting insulin dose. This process of recommending a long-acting insulin dose allows the glucose level control system to reduce the total amount of fast-acting insulin provided.

Moreover, certain embodiments disclosed herein relate to a glucose level control system that supports multiple operating modes. In some cases, at least one operating mode may be associated with a different glycemic dysregulation condition than at least one other operating mode. For example, one operating mode may be associated with T1D, while another operating mode may be associated with T2D. In some cases, the glucose level control system may provide different medicament therapy (e.g., insulin and/or counter-regulatory agent therapy) to the subject based on the selected operating mode.

Advantageously, in certain embodiments, supporting different operating modes enables a user (e.g., a healthcare provider, parent, guardian, the subject receiving treatment, etc.) to modify the operating mode of an ambulatory medicament device, such as a glucose level control system. In some cases, the operating mode may be modified automatically. Moreover, modifying the operating mode enables different dosing modes to be supported. Advantageously, supporting different dosing modes enables a glucose level control system to be used by different types of subjects, and/or a subject under different glycemic dysregulation conditions (e.g., type 1 diabetes, type 2 diabetes, etc.).

Furthermore, some embodiments disclosed herein relate to a glucose level control system capable of alerting a user or subject when an expected dose of long-acting insulin is determined to have not been administered to the subject. For instance, a subject or user that forgets or otherwise neglects to administer a dose of long-acting insulin may be alerted by the glucose level control system to administer a recommended dose of long-acting insulin dose. Further, in some cases, the glucose level control system may modify administered therapy upon determining that a dose of LAI was or was not administered to the subject. Advantageously, alerting a user to the lack of administering of a dose of LAI may facilitate the user monitoring the subject's condition and/or the subject's effectiveness at maintaining the subject's disease.

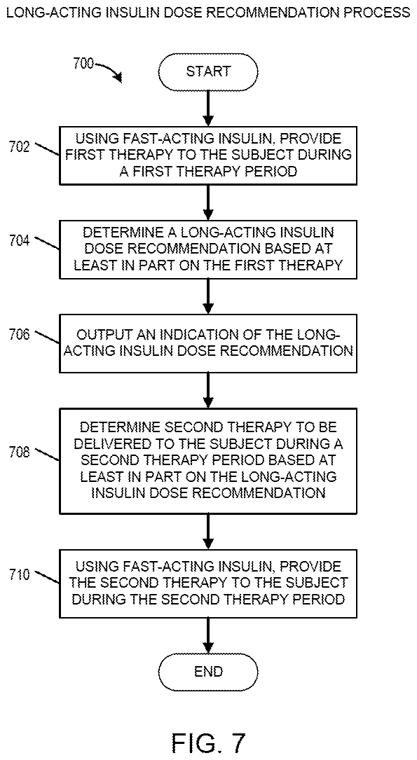

Detailed descriptions and examples of systems and methods according to one or more illustrative embodiments of the present disclosure may be found, at least, in the section entitled Long-Acting Insulin Dose Recommendation Process, and in FIGS. 7-15C and the related descriptions herein.

In some cases, a subject may manage his or her disease by using an ambulatory medicament device. The ambulatory medicament device may administer medicament based at least in part on a control algorithm. The control algorithm may determine a timing and a quantity of medicament to deliver. The ambulatory medicament device can adapt its control algorithm over time to better maintain the subject's disease. However, some subjects may not want to use the ambulatory medicament device, may not have access to the ambulatory medicament device, or may want to alternate between using the ambulatory medicament device and alternative treatment tools, such as injection therapy using a needle or a medicament pen (e.g., an insulin pen). For example, a subject may not want to be connected to an ambulatory medicament device or may not be able to afford an ambulatory medicament device.

In some cases, it may be desirable to use a glucose level control system regardless of whether the user is utilizing an ambulatory medicament device. The glucose level control system may execute a control algorithm to determine a recommended dose of medicament. In some cases, the glucose level control system generates a dose control signal to cause an ambulatory medicament device to administer the recommended dose of medicament. However, in cases where the ambulatory medicament pump is, at least temporarily, unavailable, the recommended dose of medicament may be output for display to a user enabling manual administration of the medicament via injection therapy and/or a smart pen or insulin pen. For example, the glucose level control system may output the indication of the recommended dose on a display of the glucose level control system. Thus, a user can use the glucose level control system together with injection therapy, regardless of whether the subject has access to an ambulatory medicament pump. More specifically, the user can configure a smart pen based on the displayed recommended medicament dose.

Further, the glucose level control system may transmit the indication of the recommended dose of medicament to an electronic device. For instance, the glucose level control system can transmit the recommended dose of medicament to a smart insulin pen or a smart bihormonal pen. The recommended dose can be displayed directly on the smart pen. The recommended dose may be used to automatically configure the smart pen. Additionally, in some embodiments, the glucose level control system can detect whether the recommended dose has been administered to the subject. Based on whether the recommended dose has been administered, subsequent recommended doses may be modified by the glucose level control system. In some embodiments, the glucose level control system enables a subject utilizing injection therapy or pen therapy to utilize features of the glucose level control system to improve disease management.

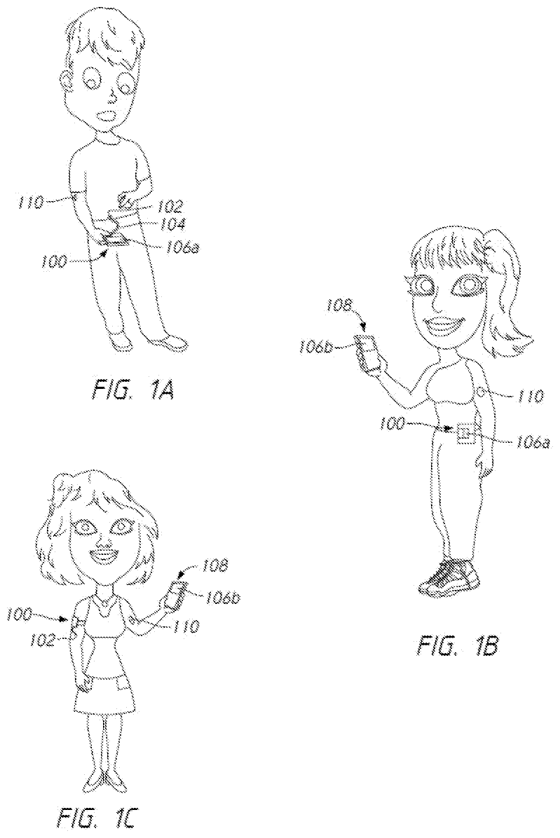

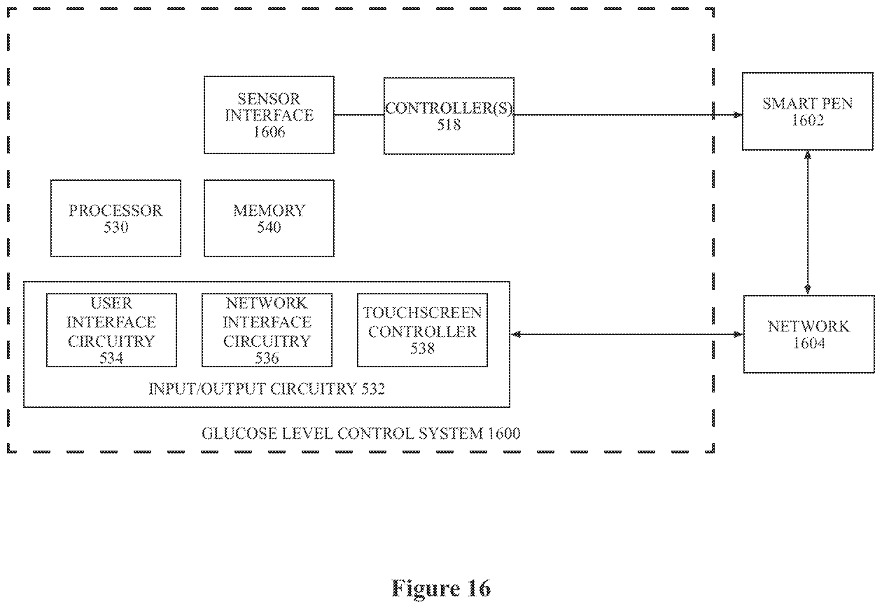

Detailed descriptions and examples of systems and methods according to one or more illustrative embodiments of the present disclosure may be found, at least, in the sections entitled Glucose Level Control System with Non-Automated Dosing Overview and Example Glucose Level Control System, and in FIGS. 16-22 and the related descriptions herein. Furthermore, components and functionality for supporting operation of an autonomous glucose level control system that may interact with a smart pen may be configured and/or incorporated into the systems and devices described with respect to FIGS. 1-6 and may be combined with one or more features described with respect to the FIGS. 7-15.

### Blood Glucose Control System Overview

Blood glucose control systems are used to control blood glucose level in a subject. Blood glucose control systems can include a controller configured to generate dose control signals for one or more glucose control agents that can be infused into the subject. Glucose control agents include regulatory agents that tend to decrease blood glucose level, such as insulin and insulin analogs, and counter-regulatory agents that tend to increase blood glucose level, such as glucagon or dextrose. A blood glucose control system configured to be used with two or more glucose control agents can generate a dose control signal for each of the agents. In some embodiments, a blood glucose control system can generate a dose control signal for an agent even though the agent may not be available for dosing via a medicament pump connected to the subject.

Glucose control agents can be delivered to a subject via subcutaneous injection, via intravenous injection, or via another suitable delivery method. In the case of blood glucose control therapy via an ambulatory medicament pump, subcutaneous injection is most common. An ambulatory medicament pump 100 is a type of ambulatory medical device, which is sometimes referred to herein as an ambulatory device, an ambulatory medicament device, a mobile ambulatory device, or an AMD. Ambulatory medical devices include ambulatory medicament pumps and other devices configured to be carried by a subject and to deliver therapy to the subject.

In some examples, the ambulatory medical device (AMD) is an electrical stimulation device, and therapy delivery includes providing electrical stimulation to a subject. An example of an electrical stimulation device is a cardiac pacemaker. A cardiac pacemaker generates electrical stimulation of the cardiac muscle to control heart rhythms. Another example of an electrical stimulation device is a deep brain stimulator to treat Parkinson's disease or movement disorders.

FIGS. 1A-1C show examples of blood glucose control systems that provide blood glucose control via an ambulatory medicament pump connected to a subject. In FIG. 1A, the medicament pump 100 is connected to an infusion site 102 using an infusion set 104. The medicament pump has integrated pump controls 106a that permit a user to view pump data and change therapy settings via user interaction with the pump controls 106a. A glucose level sensor 110 generates a glucose level signal that is received by the blood glucose control system.

In FIG. 1B, the medicament pump 100 communicates with an external electronic device 108 (such as, for example, a smartphone) via a wireless data connection. At least some of the pump controls 106a and 106b can be manipulated via user interaction with user interface elements of the external electronic device 108. The glucose level sensor 110 can also communicate with the medicament pump 100 via a wireless data connection.

In FIG. 1C, the medicament pump 100 includes an integrated cannula that inserts into the infusion site 102 without a separate infusion set. At least some of the pump controls 106b can be manipulated via user interaction with user interface elements of an external electronic device 108. In some instances, pump controls can be manipulated via user interaction with user interface elements generated by a remote computing environment (not shown), such as, for example, a cloud computing service, that connects to the medicament pump 100 via a direct or indirect electronic data connection.

Glucose control systems typically include a user interface configured to provide one or more of therapy information, glucose level information, and/or therapy control elements capable of changing therapy settings via user interaction with interface controls. The user interface can be implemented via an electronic device that includes a display and one or more buttons, switches, dials, capacitive touch interfaces, or touchscreen interfaces. In some embodiments, at least a portion of the user interface is integrated with an ambulatory medicament pump that can be tethered to a body of a subject via an infusion set configured to facilitate subcutaneous injection of one or more glucose control agents. In certain embodiments, at least a portion of the user interface is implemented via an electronic device separate from the ambulatory medicament pump, such as a smartphone.

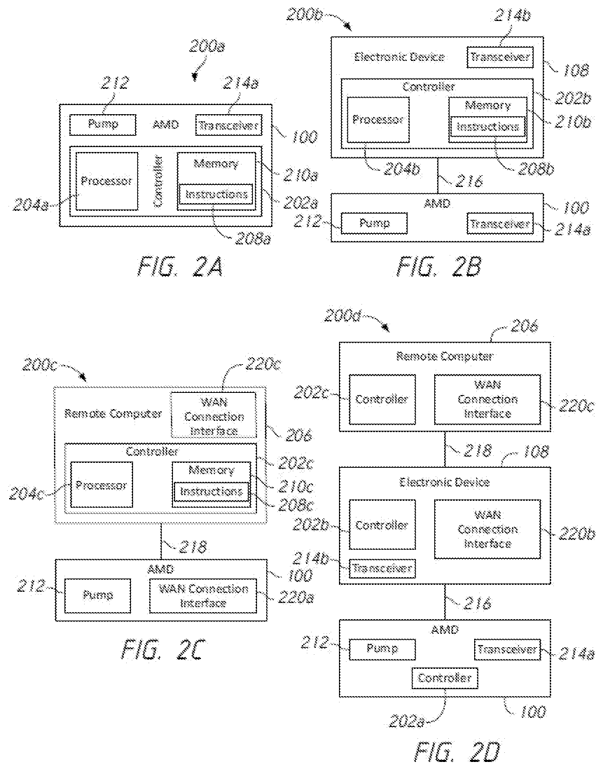

FIGS. 2A-2D illustrate block diagrams showing example configurations of a glucose control system 200. As shown in FIG. 2A, a glucose control system 200a can include a controller 202a having an electronic processor 204a and a memory 210a that stores instructions 208a executable by the processor 204a. The controller 202a and a pump 212 can be integrated with into an ambulatory medical device (AMD) 100. The AMD 100 can include a transceiver 214a for wireless digital data communications with external electronic devices. When the instructions 208a stored in memory 210a are executed by the electronic processor 204a, the controller 202a can implement at least a portion of a control algorithm that generates dose control signals for one or more glucose control agents based on time-varying glucose levels of the subject and one or more control parameters. The dose control signals, when delivered to the pump 212, result in dosing operations that control the blood glucose of a subject.

As shown in FIG. 2B, a glucose control system 200b can operate at least partially via execution of instructions 208b by an electronic processor 204b of an electronic device 108 separate from the ambulatory medical device 100. The electronic device 108 can include a transceiver 214b capable of establishing a wireless digital data connection to the AMD 100, and a controller 202b can implement at least a portion of a control algorithm via execution of instructions 208b stored in memory 210b. When the instructions 208b stored in memory 210b are executed by the electronic processor 204b, the controller 202b can implement at least a portion of a control algorithm that generates dose control signals for one or more glucose control agents based on time-varying glucose levels of the subject and one or more control parameters. The dose control signals, when delivered to the pump 212, result in dosing operations that control the blood glucose of a subject. In some embodiments, the dose control signals are transmitted from the device transceiver 214b to the AMD transceiver 214a over a short-range wireless data connection 216. The AMD 100 receives the dose control signals and passes them to the pump 212 for dosing operations.

As shown in FIG. 2C, a glucose control system 200c can operate at least partially via execution of instructions 208c on an electronic processor 204c integrated with a remote computer 206, such as, for example, a cloud service. When the instructions 208c stored in memory 210c are executed by the electronic processor 204c, the controller 202c can implement at least a portion of a control algorithm that generates dose control signals for one or more glucose control agents based on time-varying glucose levels of the subject and one or more control parameters. The dose control signals, when delivered to the pump 212, result in dosing operations that control the blood glucose of a subject. In some embodiments, the dose control signals are transmitted from the remote computer WAN connection interface 220c to the AMD WAN connection interface 220a over an end-to-end wireless data connection 218. The AMD 100 receives the dose control signals and passes them to the pump 212 for dosing operations.

As shown in FIG. 2D, a glucose control system 200d can have two or more controllers 202a, 202b, 202c that cooperate to generate a dose control signal for dosing operations by the pump 212. A remote computer 206 can transmit or receive data or instructions passed through a WAN connection interface 220c via a WAN wireless data connection 218 to a WAN connection interface 220b of an electronic device 108. The electronic device 108 can transmit or receive data or instructions passed through a transceiver 214b via a short-range wireless data connection 216 to a transceiver 214a of an AMD 100. In some embodiments, the electronic device can be omitted, and the controllers 202a, 202c of the AMD 100 and the remote computer 206 cooperate to generate dose control signals that are passed to the pump 212. In such embodiments, the AMD 100 may have its own WAN connection interface 220a to support a direct end-to-end wireless data connection to the remote computer 206.

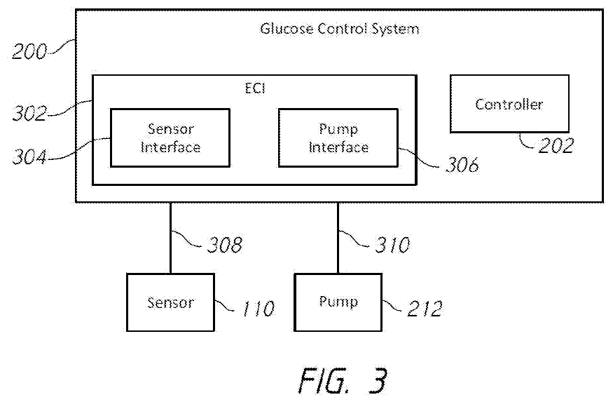

As shown in FIG. 3, in some embodiments, the glucose control system 200 includes circuitry that implements an electronic communications interface (ECI) 302 configured to send and receive electronic data from one or more electronic devices. The ECI includes a sensor interface 304 configured to receive a glucose level signal from a sensor 110 such as a continuous glucose monitor (CGM). Some CGMs generate the glucose level signal at fixed measurement intervals, such as five-minute intervals. The sensor 110 can be operatively connected to a subject in order to generate a glucose level signal that corresponds to a blood glucose estimate or measurement of the subject. The glucose level signal can be used by the controller 202 to generate a dose control signal. The dose control signal can be provided to a pump 212 via a pump interface 306. In some embodiments, the sensor interface 304 connects to the sensor 110 via a short-range wireless connection 308. In some embodiments, the pump interface 306 connects to the pump 212 via a short-range wireless connection 310. In other embodiments, the pump interface 306 connects to the pump 212 via a local data bus, such as when the controller 202, the ECI 306, and the pump 212 are integrated into an AMD 100.

The controller can be configured to generate the dose control signal using a control algorithm that generates at least one of a basal dose, a correction dose, and/or a meal dose. Examples of control algorithms that can be used to generate these doses are disclosed in U.S. Patent Application Publication Nos. 2008/0208113, 2013/0245547, 2016/0331898, and 2018/0220942 (referenced herein as the “Controller Disclosures”), the entire contents of which are incorporated by reference herein and made a part of this specification. The correction dose can include regulatory or counter-regulatory agent and can be generated using a model-predictive control (MPC) algorithm such as the one disclosed in the Controller Disclosures. The basal dose can include regulatory agent and can be generated using a basal control algorithm such as disclosed in the Controller Disclosures. The meal dose can include regulatory agent and can be generated using a meal control algorithm such as disclosed in the Controller Disclosures. Additional aspects and improvements for at least some of these controllers are disclosed herein. The dose control signal can be transmitted to an infusion motor 306 via the ECI 302 or can be transmitted to the infusion motor 306 via an electrical conductor when the controller 202a is integrated in the same housing as the infusion motor 306.

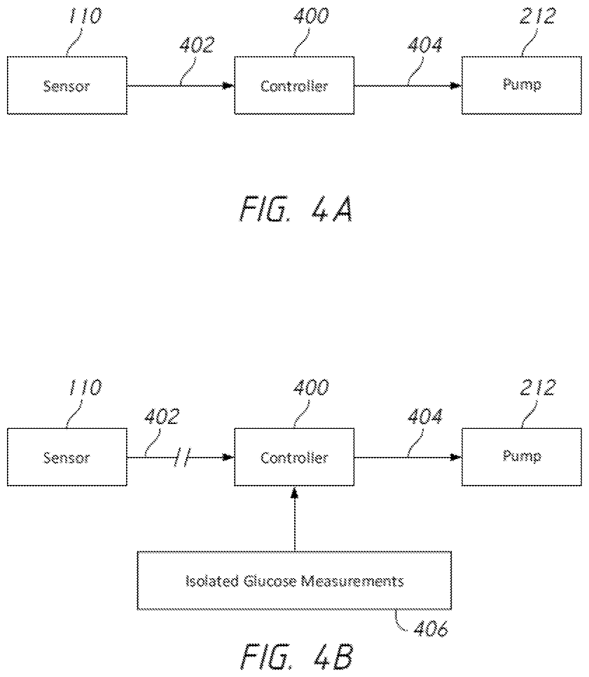

As shown in FIG. 4A, the controller 400 can be configured to operate in “online mode” during time periods when the controller receives a glucose level signal 402 from a sensor 110. In online mode, the control algorithm generates a dose control signal 404 that implements regular correction doses based on values of the glucose level signal 402 and control parameters of the control algorithm. The pump 212 is configured to deliver at least correction doses and basal doses to the subject without substantial user intervention while the controller 400 remains in online mode.

As shown in FIG. 4B, the controller 400 can be configured to operate in “offline mode” during time periods when the controller does not receive a glucose level signal 402 from a sensor 110, at least during periods when the glucose level signal 402 is expected but not received. In offline mode, the control algorithm generates a dose control signal 404 that implements correction doses in response to isolated glucose measurements 406 (such as, for example, measurements obtained from the subject using glucose test strips) and based on control parameters of the control algorithm. The pump 212 is configured to deliver basal doses to the subject without substantial user intervention and can deliver correction doses to the subject in response to isolated glucose measurements 406 while the controller 400 remains in offline mode.

### Example Implementation of Glucose Control System

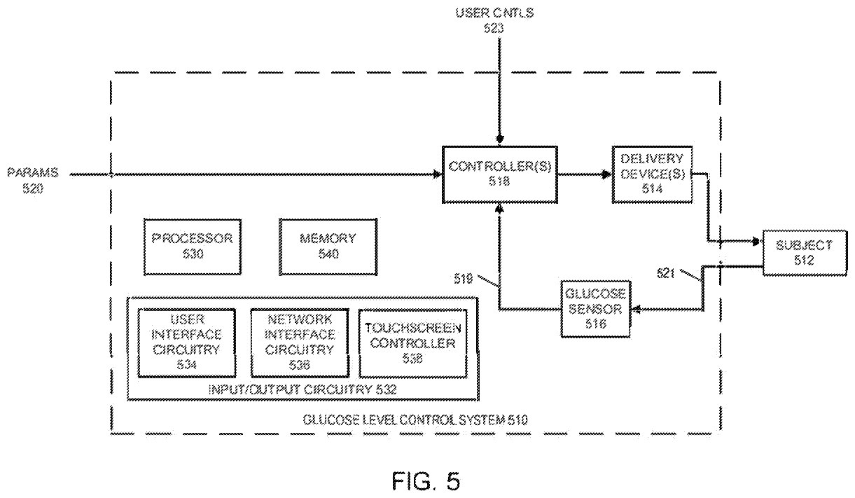

FIG. 5 illustrates an automated glucose control system 510 for regulating the blood glucose level of an animal subject (subject) 512, which may be a human. The automated glucose control system 510 is an example of a medicament infusion system and may include any of the embodiments previously described above with respect to medicament infusion systems.

The subject 512 may receive doses of insulin from one or more delivery devices 514, for example infusion pump(s) coupled by catheter(s) to a subcutaneous space of the subject 512. As described below, the delivery devices 514 may also deliver a counter-regulatory agent or hyperglycemic agent, such as glucagon or dextrose, for control of the blood glucose level under certain circumstances. For the delivery of both insulin and a counter-regulatory agent (e.g., glucagon), the delivery devices 514 may be mechanically driven infusion mechanisms having dual cartridges for insulin and the counter-regulatory agent, respectively. In the present description, reference is made to glucagon specifically, but it is to be understood that this is for convenience only and that other counter-regulatory agents (e.g., dextrose) may be used. Similarly, the term “insulin” herein is to be understood as encompassing all forms of insulin-like substances including natural human or animal insulin as well as synthetic insulin in any of a variety of forms (commonly referred to as “insulin analogs”).

For online or autonomous operation, a glucose sensor 516 is operatively coupled to the subject 512 to continually sample a glucose level of the subject 512. In some cases, the glucose sensor 516 may be referred to as a continuous glucose monitoring (CGM) sensor, which may continuously or periodically measure or sense blood glucose levels of the subject 512 for at least a period of time. Sensing may be accomplished in a variety of ways, generally involving some form of physical coupling 521 between the subject 512 and the glucose sensor 516. A controller 518 may control operation of the delivery device(s) 514 as a function of a glucose level signal 519 from the glucose sensor 516 and subject to programmed input parameters (PARAMS) 520 which may be provided by a user such as the subject 512, a parent or guardian of the subject 512, or a healthcare provider (e.g., a clinician or doctor). One input parameter for automatic operation may include the weight of the subject 512. In some cases, the glucose control system 510 can provide effective automated control without receiving explicit information regarding either meals that the subject 512 has ingested or any other “feedforward” information, which is achieved in part by an adaptive aspect to operation of the controller 518. In other cases, the glucose control system 510 can use received information regarding either meals that the subject ingested, or plans to ingest, or other “feedforward” information to modify control of blood glucose and/or delivery of insulin or counter-regulatory agent.

The controller 518 is an electrical device with control circuitry that provides operating functionality as described herein. In some embodiments, the controller 518 may be realized as a computerized device (e.g., a hardware processor) having computer instruction processing circuitry that executes one or more computer programs each including respective sets of computer instructions. In some cases, the processing circuitry will generally include one or more processors 530 along with memory 540 and input/output circuitry 532 coupled to or in communication with the processor(s) 530, where the memory 540 stores computer program instructions and data, and the input/output circuitry 532 can provide interface(s) to external devices such as the glucose sensor 516 and delivery device(s) 514. In some cases, the input/output circuitry 532 may provide a user interface, or may operate with one or more processors (e.g., the controller 518 or a separate processor 530 included in the glucose control system 510 or in a separate computing system, such as a smartphone, a laptop computer, a desktop computer, a smartwatch, and the like) to provide a user interface to a user (e.g., the subject 512, a parent or guardian, or a clinician). In some cases, the input/output circuitry 532 may include a touchscreen and/or a touchscreen controller 538 configured to control a touchscreen (not shown).

In some cases, the controller 518 may perform some or all of the functionality of the glucose level control system 510. In some such cases, the processor 530 may be optional or omitted. In other cases, the controller 518 may perform at least automated blood glucose control of the subject 512, and one or more separate processors 530 may perform one or more additional operations of the glucose level control system 510 (or medicament pump), such as tracking occurrences of hyperglycemic or hypoglycemic events or risk events, outputting data to a user, controlling or initiating communication with another computing system, regulating access to the glucose level control system 510, or other operations unrelated to operation of a medicament pump or the delivery devices 514.

The input/output circuitry 532 may control communication with one or more other computing systems and/or with a user. In some cases, the input/output circuitry 532 may include one or more separate interface circuits or controllers to facilitate user interaction and/or communication. For example, the input/output circuitry 532 may include user interface circuitry 534, network interface circuitry 536, and/or a touchscreen controller 538.

The user interface circuitry 534 may include any circuitry or processors that may output a user interface to a user and/or receive user input from the user via the user interface. The user interface circuitry 534 may receive one or more signals from a processor 530 corresponding to a user interface. The user interface circuitry 534 may control a display to present the user interface to a user based on the one or more signals received from the processor 530. Further, the user interface circuitry 534 may include any circuitry that can receive a signal corresponding to an interaction by a user with a user interface and can provide the signal to the processor 530 and/or controller 518 for further processing. In some cases, the user interface circuitry may be replaced by a touchscreen controller 538 that can control a touchscreen interface. In other cases, the touchscreen controller 538 may be in addition to the user interface circuitry 534.

The network interface circuitry 536 may include any circuitry that enables communication with a wired or wireless network. The network interface circuitry 536 may include one or more network interface cards and/or wireless radios (e.g., a Bluetooth radio, a Bluetooth Low Energy (BLE) radio, a 4g LTE radio, a 5G radio, a ND-LTE radio, and the like).

The memory 540 can include non-volatile memory and/or volatile memory. The non-volatile memory may include flash memory or solid-state memory.

The control system 510 is also able to operate in an offline manner in which it is used to provide delivery of insulin (and potentially glucagon as well), independent of or without receipt of glucose levels reported by the sensor 516. For example, in cases where the sensor 516 needs replacing, is not properly connected to the subject 512, or is defective, the glucose control system 510 may operate in an offline manner without input from the sensor 516. Thus, overall operation may be divided between online periods each including a succession of sampling intervals when a glucose signal (level) 519 is available, and offline periods each including a succession of sampling intervals when the glucose signal (level) 519 is either completely or intermittently unavailable. The description below uses the terms “online” and “offline” for these periods. Also, offline operation may be user-selected for some reason even when a glucose level signal 519 is available for use.

User control inputs (USER CNTLs 523) may be provided via a local or remote user interface of some type. In some embodiments, the user interface may resemble that of conventional insulin pumps or similar devices, e.g., by including control buttons for commanding the delivery of a bolus and perhaps a small display. In other embodiments, the system may have a wired or wireless interface to a remote device that may incorporate a fuller-function user interface, such as a smartphone, smartwatch, laptop computer, desktop computer, cloud computing service, or other wearable device or computing device. In some cases, the wireless interface may provide access to a local area network, such as a personal home network, a company network, or otherwise. Alternatively, or in addition, the wireless interface may provide a direct connection between local devices available to a user (e.g., via Bluetooth or other near field communication technologies). In some cases, the wireless interface may provide access to a wide area network, such as, but not limited to, the Internet. For example, the wireless interface may include a cellular interface that permits access to a network via a 4G or 5G cellular connection. In some cases, the cellular interface may be a low power interface, such as narrowband LTE or other Internet of Things (IoT) interfaces.

In offline mode, the glucose sensor 516 may be absent, non-functioning, or not coupled to the subject 512. As such, in offline mode, the blood glucose signal 519 may not be available to control automatic operation. In some cases, a user may provide one or more blood glucose measurements to the control system 510 to facilitate automatic operation of the control system 510. These measurements may be provided over a particular time period. Alternatively, or in addition, the glucose control system 510 may use a therapy history and/or a history of prior blood glucose control measurements to facilitate automatic operation of the control system 510 for at least a particular time period.

The description herein refers to a “user” as the source of the user control inputs 523. The “user” as used herein may be the subject 512, a parent or guardian of the subject 512, a healthcare provider (e.g., a clinician, doctor, or other person who may provide medical care to the subject), or any other user who may be authorized to help manage therapy of the subject 512. In certain implementations, the glucose level control system 510 is a personal device worn by a subject 512 for continual glucose control. In some such implementations, the user and subject 512 may be the same person. In other implementations, there may be another person involved in the care of the subject 512 and providing control input, and in such implementations, that other person has the role of user.

### Example Controllers for a Blood Glucose Control System

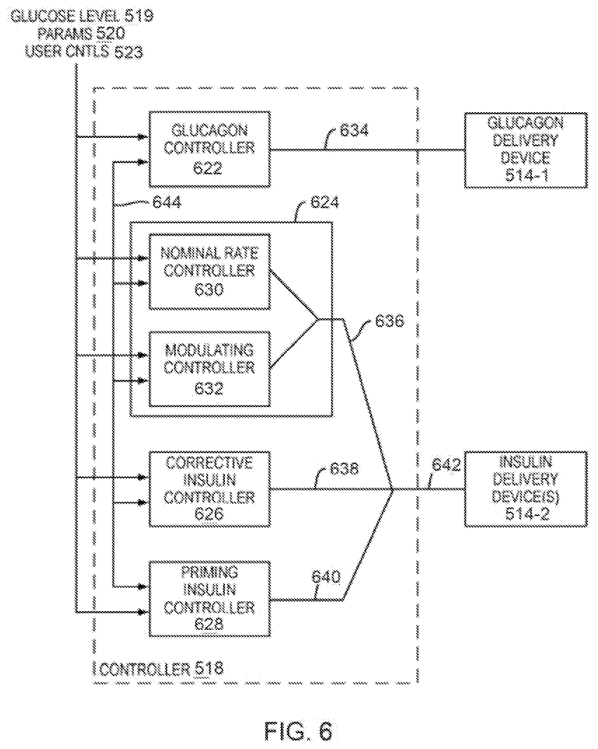

FIG. 6 shows an example structure of the controller 518 in accordance with certain embodiments. The controller 518 illustrated in FIG. 6 may represent a physical structure with different controllers or processors, or a logical structure that is implemented by one or more physical processors. In other words, a single processor may be used to implement each of the controllers illustrated in FIG. 6, each controller may be implemented by its own processor, or certain processors may implement multiple, but not necessarily all, of the controllers illustrated in FIG. 6 as part of the controller 518. Moreover, although the controllers of FIG. 6 are illustrated as part of the controller 518, in some implementations, one or more of the controllers may be separate from the controller 518.

The controller 518 may include four separate controllers, namely a glucagon (or counter-regulatory agent) controller 622, a basal insulin controller 624, a corrective insulin controller 626 (or model predictive controller), and a priming insulin controller 628 (or meal controller). The basal insulin controller 624 includes a nominal rate controller 630 and a modulating controller 632. As shown, the glucagon controller 622 generates a glucagon dose control signal 634 provided to a glucagon delivery device 514-1. Respective outputs 636-640 from the controllers 624-628 may be combined to form an overall insulin dose control signal 642 provided to insulin delivery device(s) 514-2. As shown, the output signal 636 from the basal insulin controller 624 may be formed by a combination of respective outputs of the nominal rate controller 630 and a modulating controller 632. The insulin delivery device(s) 514-2 may include devices tailored to deliver different types and/or quantities of insulin, and the exact configuration may be known to and/or under the control of the controllers 624-628. For ease of description, the collection of one or more insulin delivery devices 514-2 is referred below to in the singular as an insulin delivery device 514-2.

Also shown in FIG. 6 are input/output signals of the various controllers, including the glucose level signal 519, parameters 520 and user inputs 523 as well as a set of inter-controller signals 644. The inter-controller signals 644 enable communication of information from one controller, where the information is developed or generated, to another controller where the information may be used for that controller's control function.

The controllers 622-628 may be operated in either the online/automatic mode or in the offline mode. In the automated mode, the corrective controller 626 regulates glucose level using a control scheme such as described in U.S. Pat. No. 7,806,854, the contents of which are hereby incorporated by reference in its entirety herein. The basal controller 624 and priming insulin controller 628 may perform adaptive automated control as described in International Patent Application Publication WO 2012/058694 A2, the contents of which are hereby incorporated by reference in its entirety herein. The controllers 622-628 generally employ control methods or algorithms that include control parameters that are mathematically combined with reported glucose values to generate an output value that is converted (either directly or via additional conditioning) into the dose control signals 634, 642. For example, the control scheme described in U.S. Pat. No. 7,806,854 includes a generalized predictive control (GPC) method that incorporates a variety of control parameters. The control algorithms are generally adaptive, meaning that control parameters are dynamically adjusted during operation to reflect changing operating circumstances and a “learning” aspect—by monitoring its own operation, the algorithm adjusts its operation to be more specifically tailored to the individual user, enhancing the algorithm's effectiveness and reducing or avoiding a need for additional explicit input information about the user. It should be noted that the input parameters 520 may form part of the control parameters used by the control algorithm. Other control parameters are internal parameters according to the specifics of the algorithm, and selected ones of those internal control parameters are dynamically adjusted to realize the adaptation of the control algorithm.

One feature of operation is the ability of the controllers to learn from recent past periods of online operation and to use that learning during offline operation. U.S. Pat. No. 10,543,313, the contents of which are hereby incorporated by reference in its entirety herein, describes two methods that are usable independently or together in offline operation. A first method automatically calculates the correct size of a correction bolus of insulin at a time of receiving an isolated glucose measurement, the correction bolus then being administered by the system in response to a user control input. A second method automatically calculates the correct size of a meal bolus of insulin and administers it in response to a user control input. Both methods utilize information obtained during past periods of online operation to automatically calculate correct values, freeing the user of a need to make the calculation or provide a correction factor.

### Carbohydrate Therapy Equivalence Tracking

Hyperglycemia is a condition that occurs when the levels of sugar or glucose in the blood exceeds a particular level (e.g., 180 mg/dL). This condition may occur in diabetics. To help reduce the occurrence of hyperglycemia, a subject may use an automated blood glucose control system, which may automatically provide insulin to a subject using a medicament pump. The administered insulin may help control the blood glucose level of the subject by consuming glucose in the subject.

Hypoglycemia is a condition that occurs when the levels of sugar or glucose in the blood are below a particular level (e.g., 70 mg/dL). This condition may have adverse consequences including loss of consciousness, seizures, and death. The levels of blood sugar that lead to hyperglycemia and hypoglycemia may vary from patient to patient. To reduce the risk of hypoglycemia, a subject may consume carbohydrates to increase blood sugar. Because of the severe consequences associated with a hypoglycemic event, subjects usually consume carbohydrates that metabolize quickly. These carbohydrates are often unhealthy but are preferable to the occurrence of a hypoglycemic event. For example, the carbohydrates may include candy bars with a lot of refined sugar.

A bihormonal glucose-control system may reduce the risk of occurrence of hypoglycemia by including, in addition to insulin, a counter-regulatory agent (e.g., Glucagon) that can be administered to a subject when the blood glucose level drops too low (e.g., below 50 mg/dL). For subjects who do not have a bihormonal glucose-control system, it may be useful to understand the reduction in carbohydrate therapy, or the consumption of carbohydrates to address hypoglycemic events or potential hypoglycemic events, that can be achieved by switching to a bihormonal glucose-control system. Further, it may be useful for subjects who do have a bihormonal glucose-control system to understand the reduction in carbohydrate therapy obtained by having the bihormonal glucose-control system. For example, understanding the amount of carbohydrate therapy consumed or avoided can be important in monitoring the subject's nutrition intake. While monitoring nutrition intake is important for all people, it is particularly important for diabetics because diabetics must balance eating healthy with ensuring that their blood sugar is maintained in a particular range to avoid both hyperglycemia and hypoglycemia.

The present disclosure relates to a system that can perform a computer-implemented method of generating an indication of total carbohydrate therapy over a time period in a subject using a medicament pump configured to deliver at least insulin therapy to the subject. The system may be an automated blood glucose control system (e.g., the glucose level control system 510) that includes a hardware processor (e.g., controllers 518) for determining dose control signals to provide the medicament pump (e.g., delivery devices 514). In some cases, the medicament pump may be configured to deliver both insulin therapy and counter-regulatory agent (e.g., Glucagon) therapy. Alternatively, the system may be separate from the blood glucose control system but may receive blood glucose information from the blood glucose control system. For example, the system may be personal computing system or a cloud computing system that can received blood glucose information from the blood glucose control system.

The system may receive or determine a glucose level of a subject (e.g., subject 512). The glucose level of the subject may be determined based on a signal (e.g., a glucose level signal) received from a continuous glucose monitoring (CGM) sensor (e.g., glucose sensor 516) that corresponds to the glucose level of the subject. In some cases, the glucose level may be determined from an isolated glucose measurement, such as may be obtained using a glucose measurement kit and/or glucose paper.

Using at least the glucose level of the subject, the system can determine whether a triggering event for raising the subject's blood glucose level has occurred. The triggering event may include a blood glucose level that indicates an occurrence of a hypoglycemic event or a risk of the occurrence of a hypoglycemic event exceeding a risk threshold within a particular period of time. A risk of a hypoglycemic event may be determined when a glucose level of the subject falls below a glucose threshold. This glucose threshold may vary for different subjects and may, in some cases, be specified by the subject or a caregiver (e.g., healthcare provider, parent, or guardian). Thus, in some cases, different triggering events may be defined based on a risk tolerance of a subject to an occurrence of hypoglycemia or to possible different preferences for an amount of blood glucose to be present in the subject. Different subjects may prefer that blood glucose be maintained, or attempt to be maintained, at different levels due, for example, to differences in activity levels or metabolism by different subjects. Determining the risk of the occurrence of a hypoglycemic event may include receiving an indication of a risk of hypoglycemia from a glucose sensor or a prediction of a glucose level at a future time. For example, a determination of an imminent risk of hypoglycemia may comprise a determination that the subject's blood glucose level is expected to be below 60 mg/dL within the next 5-15 minutes.

Responsive to the triggering event, the system may determine an amount of counter-regulatory agent to administer, or an amount of counter-regulatory agent that would be administered if the blood glucose control system included the capability of administering a counter-regulatory agent. In some cases, the counter-regulatory agent is administered by, for example, the automated blood glucose control system. In other cases, the counter-regulatory agent is not administered. For example, the automated blood glucose control system may not be capable of delivering the counter-regulatory agent. As another example, the automated blood glucose control system may be capable of delivering the counter-regulatory agent but may not have a dose of the counter-regulatory agent available.

The system can use the indication of the counter-regulatory agent that is administered or that would be administered to determine a corresponding amount of carbohydrates. The corresponding amount of carbohydrates may be indicative of the amount of carbohydrates that were consumed to prevent the hypoglycemic event, to reduce the risk of the hypoglycemic event, or in response to an occurrence of a hypoglycemic event. Alternatively, or in addition, the corresponding amount of carbohydrates may be indicative of the amount of carbohydrates that would have been consumed if the counter-regulatory agent were not available.

The corresponding amount of carbohydrates may be obtained from a mapping between amounts of a counter-regulatory agent and amounts of carbohydrates. In some cases, the mapping may be based on a measured equivalency between carbohydrates and a counter-regulatory agent. Alternatively, or in addition, the mapping may be between a determined amount of counter-regulatory agent and an amount of carbohydrate a subject indicates he or she normally consumes when determining that a hypoglycemic event may occur.

The mapping may be implemented by a lookup table that maps different amounts of counter-regulatory agent to different corresponding amounts of carbohydrates. In some cases, a single quantity of counter-regulatory agent may map to different amounts of carbohydrates depending on the type of carbohydrate consumed (e.g., simple vs complex carbohydrates, or the type of candy bar consumed, etc.). Alternatively, the mapping may be based on a formula that converts an amount of counter-regulatory agent to an amount of carbohydrates based on a correspondence between the amount of counter-regulatory agent and the amount of carbohydrates. The determination of a relationship between the counter-regulatory agent and carbohydrates may be based on clinical tests comparing carbohydrates to the counter-regulatory agent (e.g., Glucagon, dextrose, etc.). Further, the mapping may be based at least in part on a subject's preferred carbohydrate source and/or characteristics of the subject (e.g., weight).

In some cases, the system can track a number of hypoglycemic events or a number of occurrences of a trigger indicating an impending risk of a hypoglycemic event within a particular time period. The time period may be days, weeks, months, years, or any other period of time over which it is desirable to determine a relationship between carbohydrates consumed or avoided based on the lack of availability or availability of a counter-regulatory agent. In some cases, the tracking of carbohydrate therapy may be based on a number of hypoglycemia events or hypoglycemia risk events instead of or in addition to a time period.

For each occurrence of a hypoglycemic event or occurrence of a trigger indicating an impending risk of a hypoglycemic event, the system can determine an estimate of the carbohydrate therapy saved or that would have been saved by having access to the counter-therapy agent. The system can generate a report for the time period that indicates the total carbohydrate saved or that would have been saved with access to counter-regulatory agent. The report may include an aggregate or sum of the carbohydrate therapy required or saved during the time period. This time period may be days, weeks, months, years, or since a particular time (e.g., since the subject starting using the system). Further, the report may indicate the type of carbohydrates typically consumed by the subject when responding to a hypoglycemic event or a risk of an impending hypoglycemic event. This report can be presented to the subject, a healthcare provider, and/or a parent or guardian of the subject. The healthcare provider can use this report to help care for the subject. For example, the healthcare provider can use the report to generate a nutrition plan for the subject that accounts for the carbohydrates consumed to maintain the blood glucose level within a desired or setpoint range.

The report may include a range of carbohydrate therapy avoided or likely consumed to address the risk of hypoglycemia events. Further, the report may include an amount of calories saved or not consumed, an amount of sugar avoided, an amount of food not consumed, a likely weight gain avoided, etc. based on the use of a counter-regulatory agent in place of carbohydrate therapy.

### Long-Acting Insulin Dose Recommendation Process

FIG. 7 presents a flowchart of an example long-acting insulin dose recommendation process 700 in accordance with certain embodiments. The process 700 may be performed by any system that can generate a long-acting insulin dose recommendation based on glucose control therapy administered to the subject. For example, the process 700 may be performed by one or more elements of the glucose level control system 510. In some cases, at least certain operations of the process 700 may be performed by a separate computing system that receives therapy data corresponding to glucose control therapy provided to the subject 512. Although one or more different systems may perform one or more operations of the process 700, to simplify discussions and not to limit the present disclosure, the process 700 is described with respect to particular systems.

The process 700 begins at block 702 where, for example, the glucose level control system 510 uses fast-acting insulin to provide first therapy to a subject 512 during a first therapy period. In some cases, the glucose level control system 510 may use a delivery device 514 to deliver the fast-acting insulin to the subject 512. The dosing of fast-acting insulin may be a basal dose or a correction bolus. The first therapy period may be a day but is not limited as such. For example, the first therapy period may be several days or weeks, or any other period of time. Additionally, the first therapy period may be a subset of a longer period (e.g., the first therapy period may include data from a particular day or week). The subset may be a sliding window (e.g., the first therapy period may include data from the most recent day or week). In some examples, the glucose level control system 510 may use a control algorithm to generate a dose control signal that autonomously causes the first therapy to be delivered to the subject. Moreover, the first therapy may include dosing of one or more additional medicaments instead of or in addition to fast-acting insulin. For example, the first therapy may include a counter-regulatory agent, such as Glucagon.

At block 704, the glucose level control system 510 determines a long-acting insulin dose recommendation based at least in part on the first therapy of fast-acting insulin provided to the subject 512. In some instances, the determination of a long-acting insulin dose recommendation may be based on the cumulative dosing of fast-acting insulin provided during the first therapy period. Alternatively, the determination of the long-acting insulin dose recommendation may be based on a fraction or portion of the dosing of fast-acting insulin provided during the first therapy period. The glucose level control system 510 may generate a recommendation of long-acting insulin based at least in part on a basal dose of fast-acting insulin provided during the first therapy period. In some cases, the recommendation may be determined based on a total daily dose or a function (e.g., an average, median, or mode, etc.) of a total daily dose of fast-acting insulin provided during the first therapy period. Moreover, the long-acting insulin dose recommendation may be based on glucose level data associated with the subject 512 received by the glucose level control system 510 (e.g., via a glucose level sensor 516 connected to the subject 512). Further, the determination of the long-acting insulin dose recommendation may be based at least in part on the type of long-acting insulin provided to the subject 512. For example, 3-day long-acting insulin may be provided to the subject 512 every three days and may be used to try and maintain the subject's 512 glucose level over the three-day period, as opposed to 1-day long-acting insulin which may be provided to the subject 512 each day and may be used to try and maintain the subject's 512 glucose level over a 24-hour period. Based on the type of LAI medicament recommended or administered to the subject, a different LAI dose may be recommended. For example, in some cases, a larger LAI dose may be recommended for 3-day LAI than for 1-day LAI. In some cases, the glucose level control system 510 may provide a recommendation for multiple types of LAI (e.g., a recommendation for both 1-day LAI and 3-day LAI) enabling a user to administer a dose of LAI based on the selected or available LAI type. Moreover, based at least in part on the type of LAI administered, the glucose level control system 510 may determine different quantities of FAI at a particular dose administration time.

In some embodiments, a long-acting insulin dose may be administered as part of the first therapy, prior to the first therapy, or within a threshold period of time. In some such cases, the glucose level control system 510 may receive an indication of the long-acting insulin dose. The glucose level control system 510 may receive the indication of the long-acting insulin dose in response to a user interaction with a user interface. Alternatively, or in addition, the glucose level control system 510 may receive the indication of the long-acting insulin dose via a wireless connection with an electronic device (e.g., a smartphone, laptop, smartglasses, tablet, server, or other remote computing device). In some cases, the wireless connection may be a direct connection between the glucose level control system 510 and the electronic device over a network. Further, if the glucose level control system 510 receives an indication that a long-acting insulin dose of a first size was administered to the subject 512 prior to the first therapy period, the long-acting insulin dose recommendation may be a recommended adjustment of the long-acting insulin dose from the first size to another size.

In other cases, the glucose level control system 510 may determine that the long-acting insulin dose has been administered based at least in part on glucose level data of the subject. In some cases, the glucose level control system 510 may determine the long-acting insulin dose recommendation based at least in part on the long-acting insulin dose administered as part of the first therapy or prior to the first therapy. Further, the long-acting insulin dose recommendation may be a recommended modification for future administration of the long-acting insulin dose previously administered as part of the first therapy or prior to the first therapy.

In some embodiments, the long-acting insulin dose recommendation may be generated based at least in part on a desired or recommended target ratio between long-acting insulin and fast-acting insulin for the subject. In some instances, the glucose level control system 510 may generate a recommendation of a dose of zero units of long-acting insulin. Alternatively, or in addition, the glucose level control system 510 may determine the long-acting insulin dose recommendation as a percentage of the total fast-acting insulin provided during the first therapy period. In some cases, the glucose level control system 510 may base the long-acting insulin dose recommendation on the amount of fast-acting insulin provided during a percentage or a portion of the first therapy period. For instance, the long-acting insulin dose recommendation may be based at least in part on the lowest basal rate provided during the first therapy period or a time period associated with the lowest basal rate.

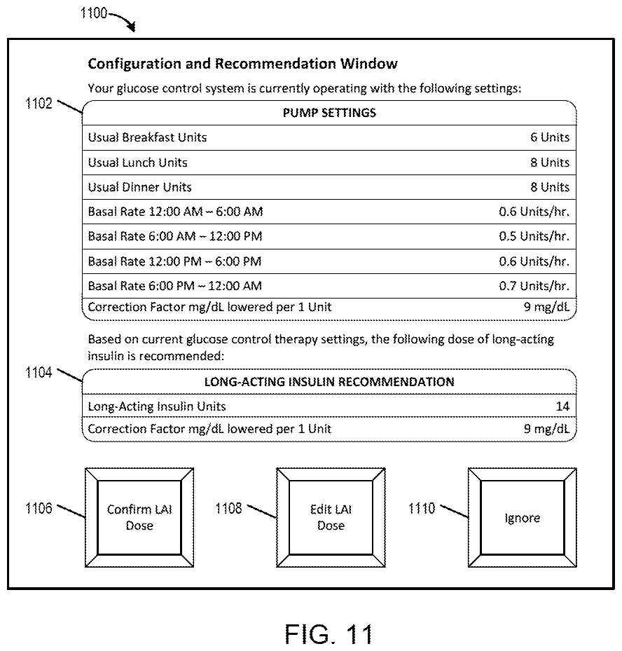

At block 706, the glucose level control system 510 may output an indication of the long-acting insulin dose recommendation. The indication of the long-acting insulin dose recommendation may be output on a display of the glucose level control system 510. In some embodiments, the glucose level control system 510 may transmit the indication of the long-acting insulin dose recommendation to an electronic device separate from the glucose level control system 510 (e.g., a smartphone, laptop, smartglasses, tablet, server, or other remote computing device), enabling the electronic device to display the indication of the long-acting insulin dose recommendation. In some cases, the electronic device may be an insulin pen that includes a display enabling the user to access the long-acting insulin dose recommendation directly on the insulin pen. As is described in more detail below with respect to FIG. 11, a user may interact with a user interface of the glucose level control system 510 or the electronic device to indicate whether the user acknowledges the recommendation, has or will administer a dose of long-acting insulin corresponding to the recommendation, has or will administer a dose of long-acting insulin that differs from the recommendation, or has decided to dismiss or ignore the long-acting insulin dose recommendation. In some cases, the user's interaction with the user interface may be recorded in a log and/or transmitted to another electronic device that may be accessible by another user (e.g., a clinician or other healthcare provider, a parent or guardian, or other authorized user). In some implementations, the block 706 may include storing the indication of the insulin dose recommendation in a memory 540 of the glucose level control system 510 and/or in a remote storage. In some cases, the long-acting insulin dose recommendation may be updated over time. For example, the process 700 may be performed repeatedly, on a particular schedule, in response to a user request, or in response to the availability of new therapy data. Subsequent performances of the process 700 may result in updated long-acting insulin dose recommendations. In some cases, updating the long-acting insulin dose recommendation may include updating a stored indication of the long-acting insulin dose recommendation in memory and/or outputting the updated long-acting insulin dose recommendation to a user.

At block 708, the glucose level control system 510 determines a second therapy to be delivered to the subject 512 during a second therapy period. The second therapy may be based at least in part on the long-acting insulin dose recommendation. The glucose level control system 510 may determine the second therapy independent of confirmation of administration of a long-acting insulin dose. In other words, the glucose level control system 510 may determine the second therapy assuming the subject is administered a long-acting insulin dose corresponding to the long-acting insulin dose recommendation. For example, the determination of the second therapy may be made at least partially in parallel as the determination of the long-acting insulin dose recommendation or in response to determining that the long-acting insulin dose has been provided to the subject 512.

Alternatively, or in addition, the glucose level control system 510 may determine whether the long-acting insulin dose corresponding to the long-acting insulin dose recommendation has been administered based at least in part on a user input and/or glucose level data obtained from a glucose level sensor. Based on the determination of the administration of the long-acting insulin dose, the glucose level control system 510 may determine the second therapy. In some cases, the glucose level control system 510 may determine a first possible second therapy based at least in part on the long-acting insulin dose recommendation and a second possible second therapy based at least in part on an assumption that long-acting insulin is not administered. The glucose level control system 510 may administer the first possible second therapy or the second possible second therapy based at least in part on a determination of whether the long-acting insulin dose is administered. The glucose level control system 510 may determine whether the long-acting insulin dose has been or is being administered to the subject 512 within a certain period of time. Further, the glucose level control system 510 may modify the determination of the second therapy responsive to modifications to the long-acting insulin dose recommendation based on updated therapy data and/or user input.

At block 710, the glucose level control system 510 uses fast-acting insulin to provide the second therapy to the subject 512 during a second therapy period. In some cases, the glucose level control system 510 may use a delivery device 514 to deliver fast-acting insulin to the subject 512. Alternatively, the second therapy may be administered by the subject 512, a medical provider, or other caretaker. The dosing of fast-acting insulin may be a basal dose or a correction bolus. The length of the second therapy period may be based on a duration of action of the recommended long-acting insulin dose. In some instances, the glucose level control system 510 may determine that zero doses of fast-acting insulin should be provided during the second therapy period. In such cases, the block 710 may be optional or omitted. For example, in some cases, the dose of long-acting insulin may fully supply the subject's insulin needs rendering the operations of the block 70 unnecessary. However, in other cases the long-acting insulin may supply a portion of the subject's insulin needs and the remaining insulin needs may be supplied by the fast-acting insulin provided as part of the second therapy. The glucose level control system 510 may use a control algorithm to generate a dose control signal that causes the second therapy to be delivered. Further, the glucose level control system 510 may be configured to autonomously cause the second therapy to be delivered using the control algorithm.

Moreover, in some examples, the glucose level control system 510 may determine the second therapy by adjusting dosing of fast-acting insulin. Adjusting the second therapy may include adjusting a basal dose or basal rate being provided to the subject 512. Such adjustment may account, at least in part, for the long-acting insulin administered to the subject 512. In some cases, adjusting the dosing of fast-acting insulin may include adjusting a basal rate to be a fraction of the basal rate applied absent the long-acting insulin dose. It should be understood that different basal rates may be provided to the subject 512 during a single day (e.g., a different rate may be provided during sleep hours than during wake hours, a different rate may be applied every 4, 6, or 12 hours, etc.). In some such cases, the adjustment of fast-acting insulin may include adjusting some or all of different basal rates.

Additionally, or alternatively, adjusting the second therapy may include adjusting one or more correction doses of insulin. In some cases, adjusting the correction doses of insulin may include reducing one or more correction doses of insulin by a particular amount or percentage. Further, adjusting the second therapy may include adjusting one or more meal or food-intake insulin doses to account for food consumption. For example, glucose level control system 510 may reduce meal doses of insulin by a particular percentage or number of insulin units.

In certain embodiments, the process 700 may be performed repeatedly. For example, the process 700 may be performed each day. Moreover, data obtained from one performance of the process 700 may be used in a subsequent performance of the process 700. For example, the second therapy administered as part of the block 710 during one iteration of the process 700 may be used as at least part of the first therapy of the block 702 for another iteration of the process 700.

### Operating Mode Selection Process

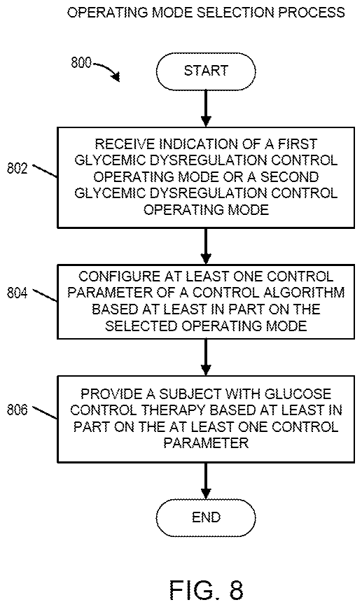

FIG. 8 presents a flowchart of an example operating mode selection process 800 in accordance with certain embodiments. The process 800 may be performed by any system capable of being configured to operate using an operating mode selected from at least a first glycemic dysregulation control (“GDC1”) mode or a second glycemic dysregulation control (“GDC2”) mode. The selected operating mode may be associated with at least one control parameter of a control algorithm used to generate a dose control signal that causes glucose control therapy to be provided to a subject. For example, the process 800 may be performed by one or more elements of the glucose level control system 510, the user controls 523, the touchscreen controller 538, the processer 530, the controller 518, the delivery device 514, and the like. In some cases, at least certain operations of the process 800 may be performed by a separate computing system that receives therapy data corresponding to glucose control therapy provided to the subject 512. Although one or more different systems may perform one or more operations of the process 800, to simplify discussions and not to limit the present disclosure, the process 800 is described with respect to particular systems.

The process 800 begins at block 802 where, for example, the glucose level control system 510 receives an indication of a GDC1 operating mode or a GDC2 operating mode. Each operating mode may be configured to support a subject 512 with one or more particular underlying conditions of glycemic dysregulation. Glycemic dysregulation is a condition that can cause an abnormality in a subject's 512 blood sugar stability. Glycemic dysregulation can be caused by a variety of conditions (e.g., T1D, T2D, prediabetes, eating disorders, malnutrition, endocrine disorders, etc.). The form of glucose control therapy used to treat a subject 512 experiencing glycemic dysregulation may vary depending on the subject's 512 underlying conditions. For example, some subjects 512 with T2D may require larger insulin doses or more frequent insulin doses to treat the subject's 512 glycemic dysregulation than some subjects 512 with T1D. The process 800 allows a user to indicate an operating mode that corresponds to the condition underlying the subject's 512 glycemic dysregulation. For example, the GDC1 operating mode may be a T1D operating mode configured to help manage a subject's type-1 diabetes. Further, the GDC2 operating mode may be a T2D operating mode configured to help manage a subject's type-2 diabetes. It should be understood that the designation of operating modes is selected for convenience and that the operating modes may be switched (e.g., the GDC1 operating mode may be configured to help manage type-2 diabetes) or may support management of other types of glycemic dysregulation conditions. Advantageously, a glucose level control system 510 with operating modes corresponding to different underlying causes of glycemic dysregulation may therefore be used to treat a variety of subjects 512 with differing underlying conditions.

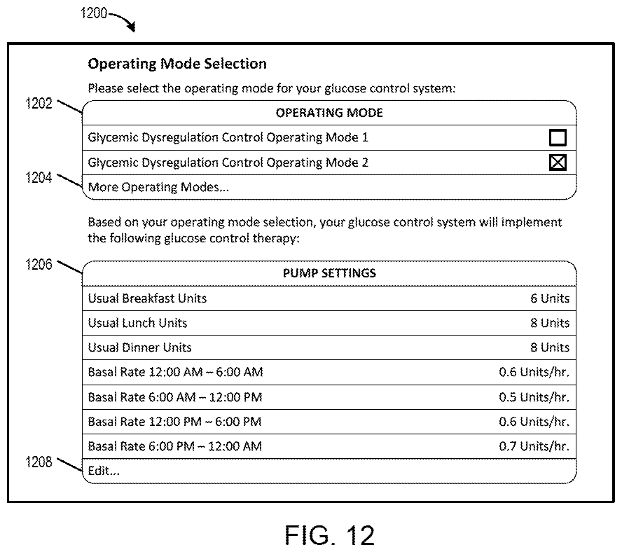

Further, as is described in more detail below with respect to FIG. 12, a user may interact with a user interface of the glucose level control system 510 (e.g., the user controls 523 or touchscreen controller 538) or an electronic device separate from the glucose level control system 510 (e.g., a smartphone, laptop, smartglasses, tablet, server, or other remote computing device) to indicate a selected operating mode. The electronic device may host an operating mode selection user interface that may be used to select an operating mode. If the user uses an electronic device separate from the glucose level control system 510 to select an operating mode (e.g., using the operating mode selection user interface), the operating mode indication may be transmitted to and received by the glucose level control system 510 via a wired or wireless connection.

At block 804, based at least in part on the selected operating mode, the glucose level control system 510 configures at least one control parameter of a control algorithm. To configure the at least one control parameter, the glucose level control system 510 may set or adjust the value of the at least one control parameter. The control parameter may be a target setpoint parameter (e.g., a glucose level target, an insulin level target, etc.); a dosing aggressiveness parameter; a glucose level prediction parameter (e.g., a parameter based at least in part on the subject's metabolism, dietary habit, biorhythm, exercise, stress, sleep, etc.); a subject model parameter (e.g., a parameter based at least in part on the subject's weight, age, puberty status, sex, geographic origin, etc.); an insulin pharmacokinetic parameter (e.g., the time at which the concentration of insulin in subject's blood reaches a maximum level (Tₘₐₓ), time when the concentration of insulin in the blood plasma reaches half of the maximum concentration (T_{1/2max}), extinction coefficient, time of action of insulin, insulin absorption rate, etc.); or a dosing setting parameter (e.g., insulin concentration, type of insulin, capacity of medicament cartridge, meal announcement dose size, food intake size, carbohydrate ratio, correction dose size, correction factor, basal rate, etc.); etc.

As stated above, configuring the at least one control parameter based on the selected operating mode of the glucose level control system 510 may include setting or modifying a target setpoint parameter or range. In some cases, the target setpoint range, or at least the upper limit or value of the target setpoint range may be set lower when the glucose level control system 510 is set or configured to operate in a T2D operating mode than when the glucose level control system 510 is set of configured to operate in the T1D operating mode. Advantageously, setting or modifying a target setpoint range based at least in part on the selected operating mode can provide for improved diabetes management because, for example, a subject with T2D may not respond to insulin therapy the same as a subject with T1D. A subject 512 with T2D may experience hysteresis such that the Beta cells secretion of insulin when the subject's glucose level increases may lag the increase in glucose level to the point where the subject's glucose level can become dangerously high or higher than desired for a healthy subject. As a type-2 diabetic may experience higher than desired glucose levels before the subject's 512 body releases insulin, it may be desirable to configure the target setpoint range such that at least the upper boundary of the target setpoint range is set to a lower setpoint value for a subject 512 with T2D enabling the glucose level control system 510 to provide insulin to the subject 512 earlier compared to a subject with T1D.

Configuring the control parameter may include setting or adjusting one or more dosing aggressiveness parameters based at least in part on the selected operating mode. Further, the dosing aggressiveness parameter may differ based at least in part on a type of insulin administered to the subject, including whether the insulin is LAI, FAI, or a combination of LAI and FAI. In some cases, an aggressiveness parameter may be configured cause more aggressive glucose control therapy when the glucose level control system 510 is configured to operate in a T2D operating mode than when configured as a T1D operating mode. Advantageously, it may be desirable to more aggressively treat or dose a subject with T2D than T1D because a subject with T2D diabetes may have a lower insulin sensitivity and therefore, require larger insulin doses, more frequent insulin doses, and/or to be dosed sooner than a subject with T1D. Further, subjects 512 with T2D may produce glucagon naturally, thus reducing the risk of hypoglycemia in subjects 512 with T2D compared to subject with T1D. Together, these factors may allow for more aggressive insulin dosing for subjects 512 with T2D than for subjects 512 with T1D. More aggressive glucose control therapy may, in some cases, include providing larger or more frequent medicament doses to the subject 512 in response to a glucose level excursion or providing medicament doses to the subject 512 in response to lower glucose level excursions.

Further, configuring the control parameter may include setting or adjusting a glucose level prediction control parameter based at least in part on the selected operating mode. For example, factors such as the subject's 512 metabolism, dietary habit, biorhythm (e.g., sleep cycle), exercise rate, or stress level may each impact the subject's glucose level or diabetes management. Some or all of these factors may directly or indirectly be associated with one or more glucose level prediction control parameters. For example, the subject's metabolism and/or dietary habit may be accounted for by one or more glucose level prediction control parameters (e.g., meal dose parameters) of the control algorithm that impact prediction of glucose level when a meal announcement is made or meal consumption is detected. As another example, a detected to reported biorhythm (e.g., a subject's typical bedtime and/or wake time) may be accounted for by one or more parameters used to set the target setpoint range (e.g., the target setpoint range may be narrower during sleep hours). Further, detected (or announced) exercise and/or detected stress levels may further affect one or more control parameters that modify glucose level prediction for a subject used to determine insulin dosing by the control algorithm of the glucose level control system 510. While each of the above factors may affect individuals differently, it should also be understood that such factors may impact a subject with T1D differently than a subject with T2D. Therefore, the glucose level control system 510 may configure one or more glucose level control parameters differently when operating in T1D mode versus T2D operating mode.

In some cases, configuring the control parameter may include setting or adjusting a subject model control parameter based at least in part on the selected operating mode. The subject model control parameter may be based at least in part on or may correspond to a subject's 512 weight, age, puberty status, sex, geographic origin or similar parameters that may impact the glucose level of the subject, the effect of insulin on the subject, or the required amount of insulin to maintain the subject's disease. For example, as a subject's 512 weight increases, the subject 512 may require more aggressive glucose control therapy. Likewise, during puberty, a subject 512 may experience higher insulin resistance, thus also requiring more aggressive glucose control therapy. While each of the above factors may affect individuals differently, it should also be understood that such factors may impact a subject with T1D differently than a subject with T2D. Therefore, the glucose level control system 510 may configure one or more subject model control parameters differently when operating in T1D mode versus T2D operating mode.

Moreover, configuring the control parameter may include setting or adjusting an insulin pharmacokinetic control parameter based at least in part on the selected operating mode. For example, factors such as Tₘₐₓ, T_{1/2max}, extinction coefficient, onset time of action of insulin, duration of action of insulin, or insulin absorption rate may each impact the timing and amount of glucose control therapy provided to a subject 512. For example, the onset time of action of insulin associated with when the administered insulin begins to reduce a glucose level of the subject may differ for different subjects. While each of the above factors may affect individuals differently, it should also be understood that such factors may impact a subject with T1D differently than a subject with T2D. Therefore, the glucose level control system 510 may configure one or more subject model control parameters differently when operating in T1D mode versus T2D operating mode.

Further, configuring the control parameter may include setting or adjusting a dosing setting control parameter based at least in part on the selected operating mode. For example, factors such as insulin concentration, type of insulin, capacity of a medicament cartridge, meal announcement dose size, food intake size, carbohydrate ratio, correction dose size, correction factor, or basal rate may impact the glucose control therapy provided to a subject 512. For instance, higher concentration insulin (e.g., U-200 vs U-100) may result in less units of medicament being supplied to the subject 512 due to the increased concentration of the medicament. Further, a subject 512 with a higher food intake may experience higher glucose levels and require a larger food-intake dose size. While at least some of the above factors may affect individuals differently, it should also be understood that some such factors may impact a subject with T1D differently than a subject with T2D. For example, all things being equal, a basal rate of a T1D subject may differ from that of a T2D subject. Therefore, the glucose level control system 510 may configure one or more subject model control parameters differently when operating in T1D mode versus T2D operating mode.

At block 806, the glucose level control system 510 provides the subject with glucose control therapy based at least in part on the at least one control parameter. In some instances, the glucose level control system 510 may use a delivery device 514 to deliver FAI to the subject 512. The dosing of FAI may be a correction bolus or a basal dose supplied with a particular basal rate. In some examples, the glucose level control system 510 may use a control algorithm to generate a dose control signal that autonomously causes the glucose control therapy to be delivered to the subject.

In certain embodiments, the process 800 may be performed repeatedly. For example, the process 800 may be performed any time the glucose level control system 510 is moved from one subject 512 to another. Advantageously, for example, the same glucose level control system 510 may first be used by a subject 512 with T1D (where the operating mode may be set to the GDC1 operating mode) and later used by another subject 512 with T2D (where the operating mode may be set to the GDC2 operating mode).

### Glucose Control Therapy Modification Process

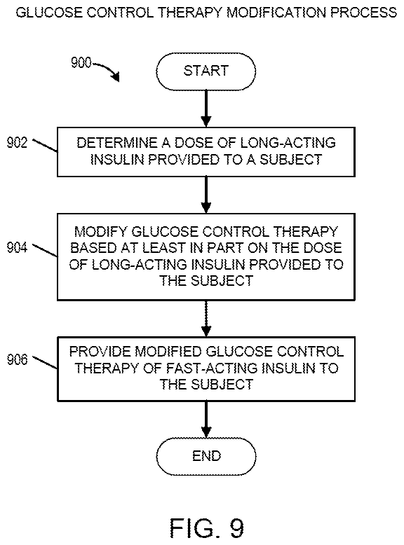

FIG. 9 presents a flowchart of an example glucose control modification process 900 in accordance with certain embodiments. The process 900 may be performed by any system that can adapt glucose level control therapy provided to a subject based at least in part on a dose of LAI provided to the subject. For example, the process 900 may be performed by one or more elements of the glucose level control system 510, the user controls 523, the touchscreen controller 538, the processor 530, the controller 518, the sensor 516, the delivery device 514, and the like. In some cases, at least certain operations of the process 900 may be performed by a separate computing system that receives therapy data corresponding to glucose control therapy provided to the subject 512. Although one or more different systems may perform one or more operations of the process 900, to simplify discussions and not to limit the present disclosure, the process 900 is described with respect to particular systems.

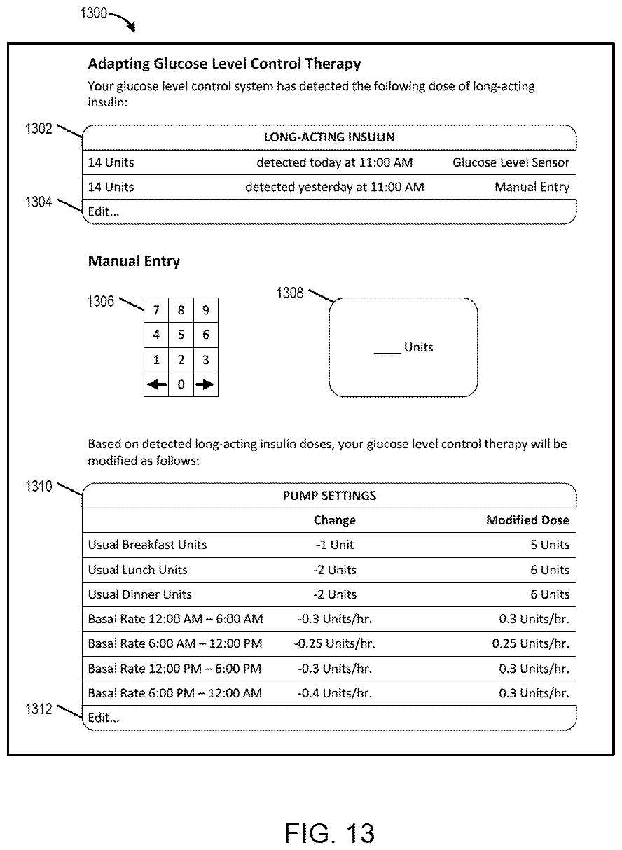

The process 900 begins at block 902, where, for example, the glucose level control system 510 determines that a dose of LAI has been provided to a subject 512. The LAI dose determination may be based at least in part on a user input signal. The user input signal may be received in response to a user interacting with a user interface of the glucose level control system 510 (e.g., the user controls 523 or touchscreen controller 538) or a user interface of an electronic device (e.g., a smartphone, laptop, smartglasses, tablet, server, or other remote computing device) in communication with the glucose level control system 510. As described in more detail below with respect to FIG. 13, a user may interact with a user interface of the glucose level control system 510 or the electronic device to indicate whether the glucose level control system's LAI dose determination is correct, edit the LAI dose determination, or manually enter a dose of LAI provided to the subject 512. In some cases, the user's interaction with the user interface may be recorded in a log and/or transmitted to another electronic device that may be accessible by another user (e.g., a clinician or other healthcare provider, a parent or guardian, or other authorized user). Further, the LAI dose determination may be based at least in part on the type of LAI provided to the subject 512. For example, 3-day LAI may be provided to the subject 512 every three days and may maintain the subject's 512 glucose level over the three-day period, as opposed to 1-day LAI which may be provided to the subject 512 each day and may maintain the subject's 512 glucose level over a 24-hour period. Based on the type of LAI medicament recommended or administered to the subject, a different LAI dose may be recommended. For example, in some cases, a larger LAI dose may be recommended for 3-day LAI than for 1-day LAI. In some cases, the glucose level control system 510 may provide a recommendation for multiple types of LAI (e.g., a recommendation for both 1-day LAI and 3-day LAI) enabling a user to administer a dose of LAI based on the selected or available LAI type. Moreover, based at least in part on the type of LAI administered, the glucose level control system 510 may determine different quantities of FAI at a particular dose administration time.

Alternatively, or in addition, the glucose level control system 510 may determine the LAI dose based at least in part on the glucose level of the subject 512 over a particular time period. The time period may be on the order of minutes to hours. The glucose level control system 510 may receive the glucose level of the subject 512 as glucose sensor data from a glucose level sensor 516 connected to the subject 512. The glucose level control system 510 may compare the glucose level of the subject 512 to an expected glucose level. The expected glucose level of the subject may be based at least in part on historical glucose data of the subject. Alternatively, or in addition, the expected glucose level of the subject may be based at least in part on an amount of one or more FAI doses provided to the subject 512 by the glucose level control system 510. The expected glucose level may be an expected value range or a specific value. If the glucose level control system 510 determines that the glucose level of the subject 512 is below the minimum of the expected value range or lower than the specific expected glucose level value by a threshold amount, the glucose level control system 510 may determine that an LAI dose was provided to the subject 512, thereby lowering the subject's 512 glucose level below the expected glucose level. The size of the LAI dose may be estimated based at least in part on the difference between the glucose level of the subject 512 and the expected glucose level of the subject.

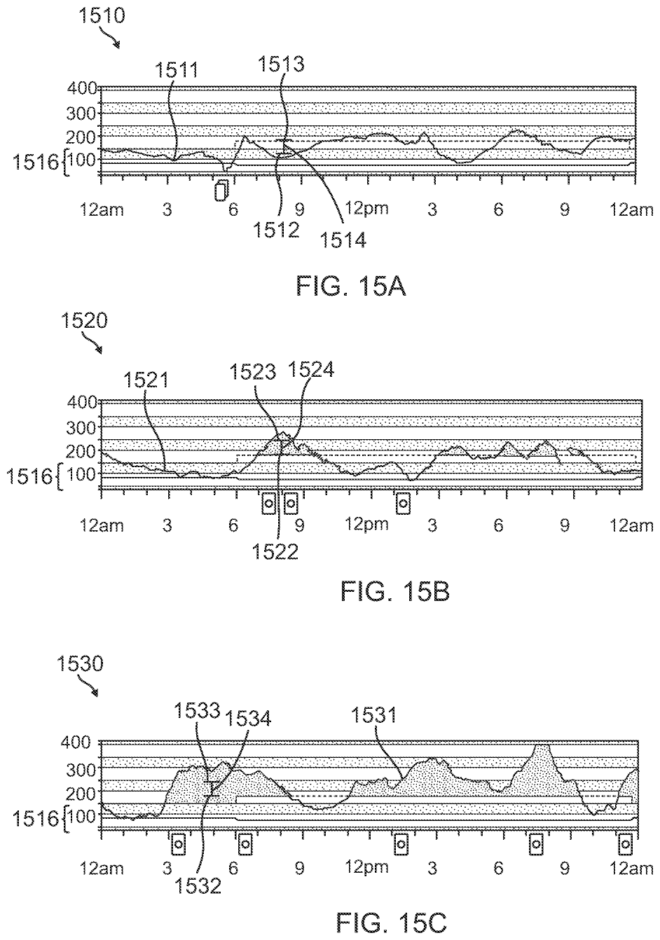

An example of how the glucose level control system 510 may determine an LAI dose based at least in part on the glucose level of a subject 512 is illustrated in Figure Graph 1510 of FIG. 15A illustrates an example of the glucose level 1511 of a subject 512 received by the glucose level control system 510 over a period of time. As illustrated, the period of time may be one day, or 24 hours. It should be understood that processes described herein are not limited to being performed on a day-by-day basis, but that the graph 1510 may illustrate one day to simplify the user interface. The range of values between the minimum expected value 1512 and the maximum expected value 1513 illustrates an expected value range of the subject's 512 glucose level 1511 at a particular point in time 1514. The expected value range between the minimum expected value 1512 and the maximum expected value 1513 may be a range of values defined as varying from a specific expected value at the point in time 1514 by a threshold amount or range. In some cases, the threshold range may be centered around the expected glucose level value (e.g., +/−10 mg/dL or +/−15 mg/dL). In other cases, the threshold range may be off-centered. For example, the threshold range may be defined as being from −5 mg/dL from the expected glucose value up to +10 mg/dL from the expected glucose value. With the glucose level 1511 of graph 1510, the glucose level control system 510 may determine that at point in time 1514, the glucose level 1511 of the subject 512 is below the minimum expected value 1512 of the expected value range between the minimum expected value 1512 and the maximum expected value 1513. The glucose level control system 510 may determine that a dose of LAI was provided to the subject 512, which caused the glucose level 1511 of the subject 512 to fall below the minimum expected value 1512 of the expected value range between the minimum expected value 1512 and the maximum expected value 1513. This LAI dose may be an unreported dose of LAI or may be a larger than reported dose of LAI. Further, as illustrated, the expected value range of the glucose level may be at least partially unrelated to the desired setpoint range 1516. Moreover, it should be understood that the desired setpoint range 1516 may vary throughout the day. In some cases, the setpoint is a single value (e.g., 10 mg/dL). Further, the range 1516 may represent an open loop range or a minimum range above which insulin is administered. Thus, in some cases, although the glucose level of a subject may exceed the setpoint, the glucose level control system 510 may not administer a correction dose until the glucose level of the subject exceeds the range 1516.

Further, the determination that a dose of LAI has been provided to the subject 512 may be based at least in part on a measure of FAI that has been administered to the subject 512 over the time period. For example, the glucose level control system 510 may estimate or predict a glucose level of a subject based on the measure of FAI provided to the subject 512 over a time period. The estimated glucose level may be compared to the actual glucose level of the subject. If the glucose level control system 510 determines that the glucose level of the subject 512 is lower than the estimated or predicted glucose level for the subject, the glucose level control system 510 may determine that an LAI dose was provided to the subject 512. It should be understood that a difference in the glucose level of the subject and the predicted glucose level based on the supplied FAI may be due to a variety of factors that affect the subject's glucose level including food, exercise, stress, physiological changes, etc. Thus, the predicted glucose level for the subject may be a glucose level range, and the glucose level control system 510 may determine whether the glucose level of the subject is within the predicted glucose level range. If the glucose level is below the predicted glucose level range, the glucose level control system 510 may determine that the LAI dose was administered to the subject 512. Moreover, the glucose level control system 510 may determine or estimate the size of the LAI dose based at least in part on the difference between the glucose level of the subject and the predicted glucose level or predicted glucose level range. Further, to determine whether an LAI dose was administered, the glucose level control system 510 may compare a glucose level of the subject over a time period (e.g., 15 minutes, 20, minutes, 30 minutes, an hour, etc.) to a predicted glucose level over the time period. If the glucose level is consistently (e.g., for at least 75%, 80%, or 90% of the time, etc.) below the predicted glucose level throughout the time period, then it may be determined that an LAI dose was administered.

The measure of FAI may be based at least in part on a user input signal. The user input signal may be received from a user interacting with a user interface of the glucose level control system 510 (e.g., the user controls 523 or touchscreen controller 538) or the user interface of an electronic device (e.g., a smartphone, laptop, smartglasses, tablet, server, or other remote computing device) in communication with the glucose level control system 510. In some cases, the measure of FAI may be based on glucose sensor data associated with the subject 512 received by the glucose level control system 510 from the glucose level sensor 516 over the time period. For example, the glucose level control system 510 may use the PK model to predict the measure of FAI provided to the subject 512 based on the subject's 512 glucose level.

At block 904, based at least in part on the dose of LAI provided to the subject 512, the glucose level control system 510 modifies glucose level control therapy that is being provided, or will be provided, to the subject 512.

FAI may be delivered to the subject 512 based at least in part on a control algorithm configured to autonomously generate dose control signals that cause the glucose control therapy to be provided to the subject 512. For example, the control algorithm may implement a pharmacokinetic (PK) model to predict, determine and modify the glucose level control therapy provided to a subject 512 by the glucose level control system 510. The PK model may include one or more parameters, referred to as control parameters, that may be subject specific and/or change overtime. Examples of factors and parameters that may influence the PK delay and/or the control parameters of the PK model may include, type of insulin, blood glucose level (e.g., at the insulin administration time), physiological characteristics of the subject, health condition of the subject, one or more physiological parameters of the subject, time of the administration, location at which the infusion set is placed, the amount of insulin administered and the like. The physiological characteristics may include characteristics shared among large portions of the population (e.g., weight, gender, age, etc.) as well as characteristics that may be unique or specific to the subject, or shared among few people (e.g., characteristics related to genetics). Differences between the physiologies of different subjects may result in differences in the optimal blood glucose range for each subject, or some subset of subjects. Further, the differences in physiologies may also affect the absorption of insulin into the blood plasma. In other words, different physiologies of different subjects may result in insulin absorption taking different amounts of time for different subjects. Thus, while the maximum concentration of glucose in blood plasma may occur 65 minutes after delivery of a bolus of fast-acting insulin for one subject, it may be 60 minutes or minutes for another subject.

Accordingly, in some such examples, the glucose level control system 510 may implement a method to adaptively change the one or more control parameters of the PK model used in its control algorithm to modify its predictions, in order to maintain the subject's 512 glucose level within a desired range. For example, the glucose level control system 510 may use readings from one or more sensors (e.g., the glucose level sensor 516) and/or information received from the subject (e.g., using a user interface of the glucose level control system 510), to modify one or more control parameters.

As indicated above, a glucose level control system 510 may control delivery or administering of insulin, or a counter-regulatory agent, based on a PK model and one or more glucose level measurements of the subject 512. In some examples, the PK model can be a bi-exponential PK model that may be used to estimate or determine the absorption or accumulation of subcutaneously administered insulin into blood and/or a decay rate of the insulin level in the subject's 512 blood for a given value of delivered dose of insulin. In some examples, the absorption of insulin over time according to a bi-exponential PK model may be represented by the following equation:

p(t)=KU₀(e^{-α}^{₁}^{t}−e^{-α}^{₂}^{t}) (2)

where U₀ is the subcutaneous dose in units (U), K is a scaling constant, and cu and as are time constants that may be used as the control parameters of the model. In some examples, the peak time of absorption of insulin, starting from the time that subcutaneous dose (U₀) is administered, may be referred to as Tmax and can be determined based on the following equation:

$$log \frac{( \frac{{α}_{2}}{{α}_{1}} )}{( {α}_{2} - {α}_{1} )} \qquad ( 3 )$$

In some examples, α₁ and α₂ can be related (e.g., through an equation such as α₂=1.5 α₁ or any other linear or nonlinear mathematical relations). In some such examples, Tmax alone may be used as the control parameter of the bi-exponential PK model. In some cases, Tmax may be the time at which the concentration of insulin in subject's blood reaches a maximum level (e.g., starting from the time that subcutaneous dose is administered). In some other examples, the bi-exponential PK model may be used to estimate or determine the accumulation of counter-regulatory agent or hormone (e.g., glucagon) in subject's blood. Equation 2 may be used to calculate the pending effect of the accumulated amount of insulin in the subcutaneously administered dose, as that can be taken to be the difference between the total area (∫₀^{∞}p(t)dt, which can describe a measure of the total amount of hormone (e.g., insulin) that can be absorbed due to a dose U₀) and ∫₀^{t}p(t)dt, which can represent a measure of the expended portion of U₀ at time.

Often, the glucose level control system 510 is configured to maintain a subject's 512 glucose level within a particular range (e.g., a normal range). As the subject's 512 glucose level rises or falls, the glucose level control system 510 may administer particular amounts of insulin or counter-regulatory agent to the subject 512 to bring the subject's 512 glucose level back to within a desired range or closer to a desired setpoint. As explained above, it may take some non-infinitesimal amount of time for the medicament to be absorbed into the subject's 512 blood stream. Thus, a PK model (e.g., the bi-exponential PK model), may be used to determine how much insulin or counter-regulatory agent should be provided to the subject 512 in order to maintain the subject's 512 glucose level within a particular range. In some examples, the PK model (e.g., the bi-exponential PK model) may be used to predict the concentration of insulin blood glucose level of the subject 512 over time as insulin or counter-regulatory agent is administered. In some cases, the control parameter values of the PK model may be set by a healthcare provider based on default values obtained through clinical trials and/or based an individualized treatment plan for the subject 512 as may be determined based on clinical tests of the subject and/or on the healthcare provider's evaluation of the subject 512, which may be determined based on tests of the subject 512.

In some embodiments, the glucose level control system 510 may modify the glucose control therapy provided to the subject 512 by adapting the control parameters of the control algorithm based at least in part on the LAI dose determination. For example, the glucose level control system 510 may change values of control parameters of the control algorithm corresponding to insulin type or insulin absorption time based at least in part on whether LAI, FAI, or a combination of LAI and FAI is administered. The glucose level control system 510 may also provide an indication of the LAI dose determination to an insulin dosing controller configured to use the control algorithm to generate an insulin dose control signal. The dose control signal may be based at least in part on glucose level data associated with the subject 512 and on the indication of the LAI dose determination.

Also, the glucose level control system 510 may provide modified glucose control therapy to the subject 512 by determining a modified basal rate of FAI based at least in part on a glucose level signal received from a glucose level sensor associated with the subject 512. The glucose level signal may reflect a modification to the glucose level of the subject 512 due in part to the dose of LAI provided to the subject. For example, the glucose level control system 510 may receive a glucose level signal from a glucose level sensor. From the glucose level signal, the glucose level control system 510 may determine that a dose of LAI has been provided to the subject 512 because the levels of glucose present in the subject 512 are inconsistent with a basal dosing of FAI alone. The glucose level control system may account for this discrepancy by determining that LAI was provided to the subject 512 and may respond by adapting one or more control parameters of the control algorithm of the glucose level control system 510.

The glucose level control system 510 may modify the glucose control therapy provided to the subject 512 by reducing one or more doses of FAI provided to the subject 512 by a particular reduction factor. The glucose level control system 510 may determine a basal rate of FAI for the subject 512 and may reduce the basal rate by the reduction factor. The reduction factor may be based at least in part on the LAI dose determination. The reduction factor may be a particular percentage or ratio (e.g., 3/4, 1/2, 1/3, etc.).

At block 906, the glucose level control system 510 provides the modified glucose control therapy to the subject 512 during a therapy period. In some instances, the glucose level control system 510 may use a delivery device 514 to deliver insulin to the subject 512. The modified glucose control therapy may include providing FAI to the subject 512.

Additionally, or alternatively, the glucose level control system 510 may determine an LAI dose recommendation based at least in part on the modified glucose control therapy delivered to the subject 512 during the therapy period. The LAI dose recommendation may include a recommended modification to a dose of LAI previously provided to the subject 512 or a recommended modification to a prior LAI dose recommendation. The glucose level control system 510 may further be configured to output the LAI dose recommendation. For example, the glucose level control system 510 may provide an LAI dose recommendation in accordance with the process 700 described above. In such an embodiment. The modified glucose control therapy provided at block 906 may be the first therapy provided to the subject 512 at block 702.

In certain embodiments, the process 900 may be performed repeatedly. For example, the process 900 may be performed each day. Moreover, data obtained from one performance of the process 900 may be used in a subsequent performance of the process 900. For example, the determination made during one iteration of the process 900 at block 902 (that a dose of LAI was provided to the subject 512) may be used during another iteration of the process 900 at block 904 to modify the glucose control therapy provided to the subject 512.

### Long-Acting Insulin Dosing Determination Process

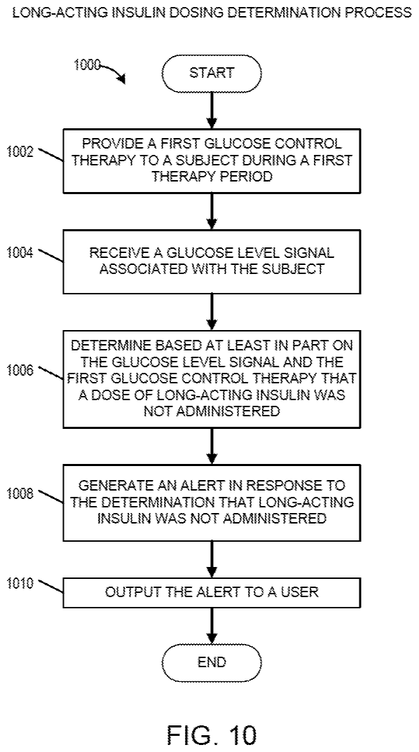

FIG. 10 presents a flowchart of an example long-acting insulin dosing determination process 1000 in accordance with certain embodiments. The process 1000 may be performed by any system that can provide glucose control therapy to a subject, receive a glucose level signal associated with the subject, determine based at least in part on the glucose level signal and glucose control therapy provided that a dose of LAI was not administered to the subject, generate an alert, and output the alert to a user. For example, the process 1000 may be performed by one or more elements of the glucose level control system 510. In some cases, at least certain operations of the process 1000 may be performed by a separate computing system that receives therapy data corresponding to glucose control therapy provided to the subject 512. Although one or more different systems may perform one or more operations of the process 1000, to simplify discussions and not to limit the present disclosure, the process 1000 is described with respect to particular systems.

The process begins at block 1002 where, for example, the glucose level control system 510 provides a first glucose control therapy to a subject 512 during a first therapy period. In some cases, the glucose level control system 510 may use a delivery device 514 to deliver FAI to the subject 512. The dosing of FAI may be a basal dose or a correction bolus. The first therapy period may be a day, but is not limited as such. For example, the first therapy period may be several days or weeks, or any other period of time. Additionally, the first therapy period may be a subset of a longer period (e.g., the first therapy period may include data from a particular day or week). The subset may be a sliding window (e.g., the first therapy period may include data from the most recent day or week). In some examples, the glucose level control system 510 may use a control algorithm to generate a dose control signal that autonomously causes the first glucose control therapy to be delivered to the subject. Moreover, the first glucose control therapy may include dosing of one or more other medicaments instead of or in addition to FAI. For example, the first glucose control therapy may include a counter-regulatory agent, such as Glucagon.

At block 1004, the glucose level control system 510 receives a glucose level signal associated with the subject 512. The glucose level control system 510 may receive the glucose level signal from a glucose level sensor 516 connected to the subject 512.

At block 1006, the glucose level control system 510 determines based at least in part on the glucose level signal and/or the first glucose control therapy whether a dose of LAI was administered to the subject 512. For example, the glucose level control system 510 may compare the glucose level of the subject 512 to an expected glucose level. Alternatively, or in addition, the glucose level control system may compare an administered amount of FAI provided to the subject 512 during the first therapy period to an expected amount of FAI predicted to be administer to the subject 512. The expected amount of FAI may be based on historical therapy for the subject over a prior time period. The expected glucose level may be based at least in part on an amount of FAI provided to the subject 512 by the glucose level control system 510. The expected glucose level may be a range of values. In some cases, the block 1006 may include predicting or determining a probability that an LAI dose was administered to the subject 512.

In some instances, the glucose level control system 510 may determine that a dose of LAI was not administered to the subject 512. For example, the glucose level control system 510 may determine from the first glucose therapy that FAI was provided to the subject 512 in excess of the expected amount of FAI. The glucose level control system 510 may determine that FAI provided to the subject 512 above the expected amount of FAI was provided in place of an LAI dose and that no LAI was provided to the subject 512. Alternatively, or in addition, the glucose level control system 510 may determine from the subject's 512 glucose level signal that the subject's 512 glucose level is above the maximum of an expected glucose level value range or higher than a specific expected glucose level value by a threshold amount. Such a determination could indicate that a dose of LAI was not provided to the subject 512, since a dose of LAI may have lowered the subject's 512 glucose level to the expected glucose level.

Further, the glucose level control system 510 may compare a basal rate of FAI delivered to the subject 512 (e.g., a basal rate from the first therapy period) to the basal rate of FAI delivered during an earlier therapy period prior to the first therapy period or to a baseline basal profile. If such a comparison shows that the basal rate is higher than the basal rate during prior therapy period or higher than the baseline basal profile, the glucose level control system 510 may determine that additional FAI was provided to the subject 512 in place of an LAI dose and that no LAI was administered to the subject 512. The baseline basal profile may be generated based at least in part on glucose therapy data associated with glucose control therapy provided to the subject 512 during the first therapy period or a therapy period prior to the first therapy period. Alternatively, or in addition, the baseline basal profile may be based on the physiological characteristics of the subject 512. The baseline basal profile may be generated based on glucose therapy data of one or more subjects 512 that share one or more physiological characteristics (e.g., age, weight, gender, puberty status, age, etc.) with the subject 512. Further, as described below, the glucose level control system 510 may detect when a physiological characteristic of the subject 512 has changed, and the glucose level control system 510 may use such a determination to generate the baseline basal profile.

The glucose level control system 510 may, based at least in part on the determination that no LAI has been provided to the subject 512, modify the glucose level control therapy that is being provided, or will be provided, to the subject 512. For instance, the glucose level control system 510 may modify the glucose control therapy provided to the subject 512 by adapting the control parameters of the control algorithm based at least in part on the determination that no LAI dose has been provided.

An example of how the glucose level control system 510 may modify the glucose level control therapy provided to a subject 512 based at least in part on the determination that no LAI has been provided to the subject 512 is illustrated in FIG. 15B. Graph 1520 of FIG. 15B illustrates an example of the glucose level 1521 of a subject 512 received by the glucose level control system 510 over a period of time. As illustrated, the period of time may be one day, or 24 hours. It should be understood that the processes described herein are not limited to being performed on a day-by-day basis, but that the graph 1520 may illustrate one day to simplify the user interface. The range of values between the minimum expected value 1522 and the maximum expected value 1523 illustrates an expected value range of the subject's 512 glucose level 1521 at a particular point in time 1524. The expected value range between the minimum expected value 1522 and the maximum expected value 1523 may be a range of values defined as varying from a specific expected value at the point in time 1524 by a threshold amount or range. In some cases, the threshold range may be centered around the expected glucose level value (e.g., +/−5 mg/dL or +/−15 mg/dL). In other cases, the threshold range may be off-centered. For example, the threshold range may be defined as being from −15 mg/dL from the expected glucose value up to +20 mg/dL from the expected glucose value. With the glucose level 1521 of graph 1520, the glucose level control system 510 may determine that at point in time 1524 the glucose level 1521 of the subject 512 is above the maximum 1523 of the expected value range between the minimum expected value 1522 and the maximum expected value 1523. The glucose level control system 510 may determine that no dose of LAI was provided to the subject 512, which caused the glucose level 1521 of the subject 512 to rise above the maximum 1523 of the expected value range between the minimum expected value 1522 and the maximum expected value 1523. In response, the glucose level control system 510 may modify the glucose level control therapy being provided to the subject 512 (e.g., by increasing the basal rate of FAI), thereby lowering the glucose level 1521 of the subject 512 to within the setpoint range 1516 over time after the point in time 1524 (e.g., due to the increased insulin provided to the subject 512). In addition to modifying the basal rate, the glucose level control system 510 may cause a correction bolus of insulin to be administered to more quickly bring the subject's glucose level with the target setpoint range 1516.

However, in other examples, the glucose level control system 510 may determine that no LAI dose has been provided to the subject 512 and may not modify the glucose control therapy provided to the subject 512. As described below, if an LAI dose was expected to be administered, the glucose level control system 510 may generate an alert in response to a determination that a dose of LAI was not administered to the subject 512.

An example of how the glucose level control system 510 may alert a user based at least in part on the determination that LAI has not been provided to the subject 512 or that a duration of action of previously provided LAI has expired is illustrated in FIG. 15C. Graph 1530 of FIG. 15C illustrates an example of the glucose level 1531 of a subject 512 received by the glucose level control system 510 over a period of time. As illustrated, the period of time may be one day, or 24 hours. It should be understood that processes described herein are not limited to being performed on a day-by-day basis, but that the graph 1530 may illustrate one day to simplify the user interface. The range of values between the minimum expected value 1532 and the maximum expected value 1533 illustrates an expected value range of the subject's 512 glucose level 1531 at a particular point in time 1534. The expected value range between the minimum expected value 1532 and the maximum expected value 1533 may be a range of values defined as varying from a specific expected value at the point in time 1534 by a threshold amount or range. In some cases, the threshold range may be centered around the expected glucose level value (e.g., +/−12 mg/dL or +/−20 mg/dL). In other cases, the threshold range may be off-centered. For example, the threshold range may be defined as being from −8 mg/dL from the expected glucose value up to +12 mg/dL from the expected glucose value. With the glucose level 1531 of graph 1530, the glucose level control system 510 may determine that at point 1534, the glucose level 1531 of the subject 512 is above the maximum 1533 of the expected value range between the minimum expected value 1532 and the maximum expected value 1533. The glucose level control system 510 may determine that a dose of LAI was not provided to the subject 512 or that a duration of action of a provided dose of LAI has expired based on a determination that the glucose level 1531 of the subject 512 has risen above the maximum 1533 of the expected value range between the minimum expected value 1532 and the maximum expected value 1533. In response to determining that the glucose level exceeds an expected glucose level range, the glucose level control system 510 may, as described below, generate and/or output an alert notifying a user that an LAI dose was not provided to the subject 512 of that a duration of action of a provided dose of LAI has expired. In some cases, the alert may indicate that a provided dose of LAI is less than recommended or less than reported.

In some embodiments of the process 1000, the glucose level control system 510 may detect, based at least in part on the glucose level signal and the first glucose control therapy, whether a physiological condition of the subject 512 has changed. For example, the glucose level control system may determine that the subject 512 is ill based at least in part on the subject's 512 glycemic response to the first glucose control therapy. For example, the glucose level control system 510 may store in a memory 540 the subject's 512 glycemic response to prior glucose control therapy. If the glucose level control system 510 determines based at least in part on the glucose level signal or glucose level data that the subject's 512 response to the first glucose control therapy differs from the response to prior glucose control therapy, the glucose level control system may determine or predict that the subject 512 is ill. Since a change in physiological condition (e.g., illness) may impact the subject's 512 glucose level and/or response to glucose control therapy, it is advantageous for the glucose level control system to be capable of determining such changes. The glucose level control system 510 may generate an alert in response to determining that there has been a change in the subject's 512 physiological condition. The glucose level control system 510 may alternatively, or in addition, change a control parameter or adjust a control algorithm to account for the change detected in the subject's 512 physiological condition, thus personalizing the glucose control therapy the glucose level control system 510 provides to the subject 512.

Further, the glucose level control system 510 may be capable of determining whether a dose of LAI administered to the subject 512 is too high or too low. In some examples of the process 1000, the glucose level control system 510 may compare the ratio of the determined LAI dose (or lack of a LAI dose) to FAI provided by the glucose level control system 510 to an expected ratio of LAI to FAI for a time period. The expected ratio may be set by a healthcare provider or other user. As daily insulin use may vary, the expected ratio may be a range of ratio values. Alternatively, or in addition, the glucose level control system 510 may determine whether an LAI to FAI ratio is within a threshold degree of an expected ratio. Alternatively, or in addition, the LAI to FAI ratio may be determined from prior therapy periods. If the ratio of the determined LAI dose to cumulative FAI for the time period is higher than the expected ratio of LAI to FAI, the glucose level control system may determine that the determined dose of LAI administered to the subject 512 was too high for the time period. Conversely, if the determined ratio is lower than the expected ratio, the glucose level control system may determine that the determined dose of LAI administered to the subject 512 was too low. Alternatively, or in addition, the glucose level control system 510 may determine whether the dose of LAI was too high or too low a dosage by comparing the dose of LAI to a recommended dose of LAI (e.g., the LAI dose recommendation of block 704) to determine whether the dose of LAI exceeds the recommended dose of LAI. In some instances, the glucose level control system 510 may determine whether the LAI dose administered to the subject 512 exceeded the recommended dose of LAI by a threshold amount.

In some examples of the process 1000, the glucose level control system 510 may account for counter-regulatory agent (e.g., Glucagon) provided to the subject 512 when determining whether a dose of LAI was provided to the subject 512. For example, the glucose level control system 510 may account for counter-regulatory agent provided to the subject 512 by modifying an expected FAI dosing regimen. Alternatively, or in addition, the glucose level control system 510 may account for counter-regulatory agent provided to the subject 512 by adjusting an expected LAI to FAI ratio.

At block 1008, the glucose level control system 510 generates an alert in response to a determination that a dose of LAI was not administered to the subject 512. The alert may include a reminder for the subject 512 to take an expected or recommended dose of LAI. Alternatively, or in addition, the alert may include a notification that the glucose level control system 510 has detected a change in the subject's 512 physiological condition (e.g., that the subject 512 is ill). In some cases, the alert may be updated over time. For example, the process 1000 may be performed repeatedly, on a particular schedule, in response to a user request, or in response to the availability of new therapy data. For example, the glucose level control system may generate a daily alert. Subsequent performances of the process 1000 may result in updated alerts.

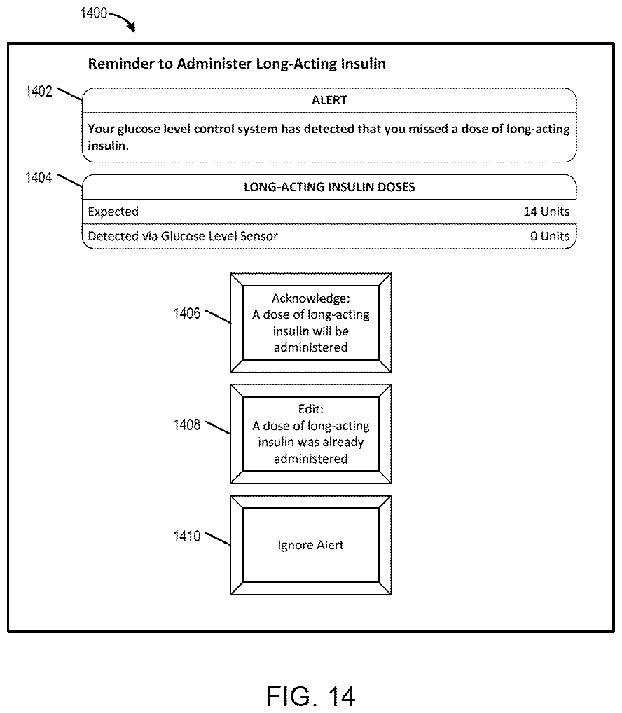

At block 1010, the glucose level control system 510 outputs the alert to a user. The alert may be output on a display of the glucose level control system 510. In some embodiments, the glucose level control system 510 may transmit the alert to an electronic device separate from the glucose level control system 510 (e.g., a smartphone, laptop, smartglasses, tablet, server, or other remote computing device), enabling the electronic device to display the alert. In some cases, the electronic device may be an insulin pen that includes a display enabling the user to access the alert directly on the insulin pen. As described in more detail below with respect to FIG. 14, a user may interact with a user interface of the glucose level control system 510 or the electronic device to indicate whether the user acknowledges the alert, wishes to edit the alert by indicating that LAI was provided to the subject 512, or has decided to dismiss or ignore the alert. In some cases, the user's interaction with the user interface may be recorded in a log and/or transmitted to another electronic device that may be accessible by another user (e.g., a clinician or other healthcare provider, a parent or guardian, or other authorized user). In some implementations, the block 1010 may include storing the alert in a memory 540 of the glucose level control system 510 and/or in a remote storage. In some cases, updating the alert may include updating a stored indication of the alert in a memory and/or outputting the updated alert to a user.

Additionally, the glucose level control system 510 may modify a second glucose control therapy to be delivered to the subject 512 during a second therapy period. The glucose level control system 510 may modify the second glucose control therapy in response to determining that LAI was not administered to the subject 512 during the first therapy period and/or that a physiological condition of the subject 512 has changed. The glucose level control system 510 may modify the second glucose control therapy by adapting a control algorithm based at least in part on the determination of an absence of an LAI dose or on the changed physiological condition of the subject 512.

For instance, if the glucose level control system 510 determines that no LAI was provided to the subject 512, or that the dose of LAI provided to the subject 512 is lower than expected, the glucose level control system 510 may determine the basal dosing rate of FAI being delivered to the subject 512 and increment the basal dosing rate by an augmentation factor. The augmentation factor may be based at least in part on a dose of LAI determined by the glucose level control system 510. Alternatively, or in addition, the augmentation factor may be based at least in part on a determination that LAI was not provided to the subject 512. The augmentation factor may include a percentage or a ratio. The augmentation factor may be based at least in part on the type of LAI provided to the subject 512. For example, 3-day LAI may be provided to the subject 512 every three days and may maintain the subject's 512 glucose level over the three-day period, as opposed to 1-day LAI which may be provided to the subject 512 each day and may maintain the subject's 512 glucose level over a 24-hour period.

In certain embodiments, the process 1000 may be performed repeatedly. For example, the process 1000 may be performed each day or every three days, etc. Moreover, data obtained from one performance of the process 1000 may be used in a subsequent performance of the process 1000. For example, the glucose level signal associated with the subject received as part of block 1004 during one iteration of the process 1000 may be used at least in part to make the determination of block 1006 that a dose of LAI was not administered to the subject 512 for another iteration of the process 1000.

### User Interface Overview

FIGS. 11-14 illustrate non-limiting examples of user interfaces relating to one or more of the LAI-based embodiments disclosed herein. The glucose level control system 510 and/or an electronic device in communication with the glucose level control system 510 may generate or display the user interface of FIGS. 11-14. Further, FIGS. 11-14 represent just one non-limiting example of user interfaces that may be generated. It is possible for other user interfaces to be generated that include more or less data and/or user interaction options such as buttons or UI elements. For example, the tables 1102 and 1104 of FIG. 11 may be displayed on two separate user interface screens that may be separately generated and/or accessed.

### Configuration and Recommendation Interface

FIG. 11 illustrates an example configuration and recommendation interface 1100 in accordance with certain embodiments. The pump settings of the glucose level control system 510 displayed in the table 1102 may indicate configuration values for one or more types of insulin doses (e.g., meal doses, basal rate doses, and/or correction doses) and may be displayed as units of insulin or as an insulin rate. Further, the table 1102 may include a correction factor indicating how much 1 unit of insulin will generally lower the blood glucose level of the subject. The interface 1100 may also display a LAI dose recommendation. The LAI dose recommendation may be based on the pump settings displayed in the table 1102. Although not illustrated, in some cases, the interface 1100 may identify the concentration of insulin included in a unit (e.g., U-100 insulin, U-200 insulin, etc.) and/or the type of insulin. Further, in some cases, the interface 1100 may be interactive enabling a user to specify a type of insulin or insulin concentration administered by the glucose level control system 510. In some such cases, the interface 1100 may update the pump settings and LAI recommendation based on the type of insulin and/or insulin concentration selected.

The table 1102 of the interface 1100 provides an example of a pump settings display. As illustrated, the table 1102 may display the pump settings under which the glucose level control system 510 is currently operating. The table 1102 may include a display of glucose control therapy corresponding to mealtimes (e.g., breakfast, lunch, or dinner). The usual-sized meal may refer to the size of a meal that the particular subject 512 usually consumes or has been advised to consume by a healthcare provider. The units of insulin associated with a usual mealtime dose may refer to an amount of insulin that the glucose level control system 510 provides the subject 512 when the subject consumes the identified usual size meal. Although not illustrated, the table 1206 may display larger or smaller units of insulin associated with a meal that is larger or smaller, respectively, than a usual or typical meal for the subject.

The table 1102 may also, or alternatively, display one or more basal rates of medicament provided to the subject 512. As illustrated, the basal rate may vary over time. In the illustrated example, a basal rate is supplied for four different time periods constituting a 24-hour day. However, the basal rate may be divided into fewer (e.g., 2 twelve-hour blocks) or greater (e.g., every four hours) number of periods, with each time period potentially having a different basal rate as determined based on the historical therapy data provided by a glucose level control system 510.

As illustrated by the table 1104, the interface 1100 may identify an amount of LAI a subject 512 is recommended to administer at a particular time (e.g., one or more particular times throughout a day) or over a period of time (e.g., over 1 day, 3 days, 5 days, a week, etc.). This LAI dose recommendation may be based on the current pump settings of the glucose level control system 510, which may be displayed in the same or a different interface.

Alternatively, or in addition, the LAI dose recommendation may be a LAI dose recommendation generated using the process 700, as described above with respect to FIG. 7. In some cases, the interface 1100 may include additional data used to generate the LAI dose recommendation of table 1104. This additional data may include cumulative dosing amounts during a therapy period, a total daily dose or a function (e.g., an average, median, or mode, etc.) of a total daily dose of FAI, the glucose level of the subject 512 at a particular time or over a period of time, a prior LAI dose that was provided to the subject 512, a target ratio of LAI to FAI, or a percentage of total FAI provided to the subject 512 during a therapy period or portion of a therapy period.

Further, the table 1102 and/or table 1104 may identify the reduction in glucose level attributable to one unit of insulin. For example, as illustrated, the glucose level control system 510 has determined that one unit of insulin (e.g., 1/100^{th} of a milliliter of insulin) may reduce a subject's 512 blood glucose level by 9 mg/dL. Accordingly, a user implementing injection therapy may measure a subject's 512 blood glucose level, determine a difference between the measured blood glucose level and a desired setpoint or threshold glucose level, and divide the difference by 9 to determine a number of units of insulin to inject in response to a determination that a correction dose is warranted (e.g., that blood glucose is outside of a desired setpoint range).

Further, the interface 1100 may include UI elements (e.g., buttons or icons, etc.) 1106, 1108, and 1110. Such UI elements may allow the user to interact with the interface 1100. For example, UI element 1106 may allow the user to indicate that the user acknowledges the LAI dose recommendation and has or will administer a dose of LAI corresponding to the LAI dose recommendation. The UI element 1108 may allow the user to indicate that the user has or will administer a LAI dose that differs from the recommendation and allow the user to input the different LAI dose (e.g., via a number pad). Further, the UI element 1110 may allow the user to indicate that the user has decided to dismiss or ignore the LAI dose recommendation. Further UI elements may include a confirmation interface asking the user to confirm the user's selection. The user's indication via the UI elements 1106, 1108, and 1110 may be recorded in a log and/or transmitted to another electronic device that may be accessible by another user (e.g., a clinician or other healthcare provider, a parent or guardian, or other authorized user).

### Operating Mode Selection Interface

FIG. 12 illustrates an example operating mode selection interface 1200 in accordance with certain embodiments. The interface 1200 may allow the glucose level control system 510 to receive a user selection of a GDC1, GDC2, or other operating mode in the table 1202. The interface 1200 may further display pump settings for the glucose level control system 510 in the table 1206 based on the operating mode selected. The pump settings displayed in the table 1206 may indicate configuration values for one or more types of insulin doses (e.g., meal doses, basal rate doses, and/or correction doses) and may be displayed as units of insulin or as an insulin rate. Further, the table 1206 may include a correction factor indicating how much 1 unit of insulin will generally lower the blood glucose level of the subject in units of mg/dL. In some cases, the interface 1200 may allow a user to edit the pump settings that are by default associated with the selected operating mode.

The table 1202 of the interface 1200 provides user interactive options that allow a user to select an operating mode of the glucose level control system 510. The table 1202 may provide options for a GDC1, GDC2, and/or other operating modes. The GDC1 and GDC2 operating modes may be those described above with respect the process 800 and FIG. 8. As illustrated, the table 1202 indicates which operating mode is currently selected by marking the active operating mode with an “X”, however, it should be appreciated that a variety of other methods may be used to indicate the active operating mode (e.g., highlighting, text labels, etc.).

Further, the table 1202 may display in a single list the full list of all operating modes available for user selection. Alternatively, the table 1202 may display only a portion of the full list of all operating modes available for user selection and provide a list expansion option 1204. The list expansion option 1204 may allow a user to view additional operating modes available for selection that were not originally displayed. The list expansion option may additionally, or alternatively, allow a user to define a new operating mode with modified pump settings, import to the glucose level control system 510 an operating mode from an electronic device, or export to an electronic device an operating mode from the glucose level control system 510. The electronic device may be another glucose level control system 510.

For example, the table 1202 may by default display a GDC1 and GDC2 operating mode for user selection. The user may select one of GDC1 or GDC2 by interacting with the table 1202. If a user does not wish to select one of GDC1 or GDC2, the user may select the list expansion option 1204 to choose another operating mode (e.g., an operating mode that has been customized by a physician for a particular subject 512).

In table 1206, the interface 1200 may display the pump settings of the glucose level control system 510 that will be used to provide glucose control therapy to the subject 512 based on the active operating mode displayed in table 1202. The pump settings may be displayed as units of insulin or as an insulin rate. As illustrated, the table 1206 may display glucose control therapy corresponding to mealtimes (e.g., breakfast, lunch, or dinner). The usual-sized meal may refer to the size of a meal that the particular subject 512 usually consumes or has been advised to consume by a healthcare provider. The units of insulin associated with a usual mealtime dose may refer to an amount of insulin that the glucose level control system 510 provides the subject 512 when the subject consumes the identified usual size meal. Although not illustrated, the table 1206 may display larger or smaller units of insulin associated with a meal that is larger or smaller, respectively, than a usual meal.

The table 1206 may also, or alternatively, display one or more basal rates of medicament provided to the subject 512. As illustrated, the basal rate may vary over time. In the illustrated example, a basal rate is supplied for four different time periods constituting a 24-hour day. However, the basal rate may be divided into fewer (e.g., 2 twelve-hour blocks) or greater (e.g., every four hours) number of periods, with each time period potentially having a different basal rate as determined based on the historical therapy data provided by a glucose level control system 510.

Further, the table 1206 may provide a modification option 1208. The modification option 1208 may allow a user to modify the pump settings displayed in table 1206. Advantageously, a glucose level control system 510 that provides a modification option 1208 may allow personalized tailoring of the glucose level control system's 510 pump settings for the needs of particular subjects 512. If the pump settings are modified to meet the glucose control therapy needs of a particular subject 512, the modified pump settings may be saved by a user as a new operating mode (e.g., by interacting with the list expansion option 1204 of table 1202).

For example, if a particular subject 512 is a type-2 diabetic, a user may select an operating mode in table 1202 that generally configures the glucose level control system with pump settings appropriate to treat type-2 diabetes. Such pump settings may be displayed in table 1206 and may indicate configuration values for one or more types of insulin doses (e.g., meal doses, basal rate doses, and/or correction doses). In some cases, the pump settings generated by the glucose level control system operating under the selected operating mode may be sufficient for the subject's 512 needs. However, in some instances, the pump settings displayed in table 1206 may be interpreted as a baseline recommendation for FAI glucose control therapy, and a user (e.g., a physician or other healthcare provider) may review the pump settings and use the modification option 1208 to tailor the pump settings to the particular subject's 512 glucose control therapy needs if such needs are different than the baseline recommendation. The user may then save the modified pump settings as a new operating mode by interacting with the list expansion option 1204. Advantageously, if the subject 512 later needs a replacement glucose level control system 510 (e.g., due to a mechanical failure of the original glucose level control system 510), the user may export the new operating mode containing the modified pump settings, import the new operating mode onto a replacement glucose level control system 510, and select the new operating mode in table 1202 of the replacement glucose level control system 510. The pump settings of the replacement glucose level control system 510 may thereby be made identical to the pump settings of the original glucose level control system 510, thus reducing the time needed to configure the replacement glucose level control system 510 for the subject 512.

### Adapting Glucose Level Control Therapy Interface

FIG. 13 illustrates an example adapting glucose level control therapy interface 1300 in accordance with certain embodiments. The interface 1300 can display in table 1302 LAI doses that the glucose level control system 510 determines have been provided to the subject 512. The interface 1300 may further display in table 1310 modifications to glucose control therapy for the subject 512 based at least in part on a determined LAI dose provided to the subject 512. In some cases, the interface 1300 may identify the concentration of insulin included in a unit of insulin (e.g., U-100 insulin, U-200 insulin, etc.) and/or the type of insulin. Further, in some cases, the interface 1300 may be interactive and allow a user to edit the glucose level control system's 510 determination that LAI has been provided, indicate that LAI has been provided to the subject 512, or modify the glucose level control system's 510 response to determining that LAI has been provided to the subject 512.

The table 1302 provides an example of an LAI dose determination display. The LAI dose determination displayed may be the LAI dose determination discussed above with respect to the process 900 and FIG. 9. As illustrated, the table 1302 may display doses of LAI that the glucose level control system 510 determines were provided to the subject 512. The table 1302 may display the LAI doses in units of insulin. Further, the table 1302 may display the date and time at which the glucose level control system 510 detected that the LAI dose was provided to the subject 512. For each detected dose of LAI, the table 1302 may display the source of its determination that LAI was provided to the subject 512 (e.g., a glucose level sensor 516, manual entry by a user, etc.).

In some cases, the interface 1300 may include additional data used to determine that LAI was provided to the subject 512. This additional data may include the glucose level of the subject 512 at a particular time or over a time period, glucose sensor data received from a glucose level sensor 516, a measure of FAI that has been provided to the subject 512 over a time period, or a LAI dose recommendation (e.g., as described with respect to the process 700 and FIG. 7).

The table 1302 may have an LAI dose determination modification option 1304. The LAI dose determination modification option 1304 may allow a user to modify the LAI dose determination(s) displayed in table 1302. For example, a user may see in table 1302 that the glucose level control system 510 has determined that a dose of LAI consisting of 14 units of insulin was provided to the subject at a particular time and date. The user may know, however, that only 12 units of insulin were provided. It may therefore be advantageous to allow the user to modify the determination to correct the dosage amount.

Alternatively, or in addition, the interface 1300 may allow a user to manually indicate that a dose of LAI was provided to the subject 512. The interface 1300 may include a virtual keypad 1306 or other user entry display to allow a user to enter the dosage of LAI provided to the subject. The entered LAI dose may appear in a confirmation interface 1308. The confirmation interface 1308 may allow the user to confirm that the LAI dose the user entered is correct before saving the dose information. Once entered, the LAI dose information may be added to the table 1302.

Table 1310 of the interface 1300 may display the pump settings of the glucose level control system 510 that will be used to provide glucose control therapy to the subject 512 based on the LAI doses determination(s) displayed in table 1302. The pump settings may be displayed as units of insulin or as an insulin rate. As illustrated, the table 1310 may include a display of glucose control therapy corresponding to mealtimes (e.g., breakfast, lunch, or dinner). The usual-sized meal may refer to the size of a meal that the particular subject 512 usually consumes or has been advised to consume by a healthcare provider. The units of insulin associated with a usual mealtime dose may refer to an amount of insulin that the glucose level control system 510 provides the subject 512 on average when the subject consumes the identified usual size meal. Although not illustrated, the table 1206 may display larger or smaller units of insulin associated with a meal that is larger or smaller, respectively, than a usual meal.

Table 1310 may also, or alternatively, display one or more basal rates of medicament provided to the subject 512. As illustrated, the basal rate may vary over time. In the illustrated example, a basal rate is supplied for four different time periods constituting a 24-hour day. However, the basal rate may be divided into fewer (e.g., 2 twelve-hour blocks) or greater (e.g., every four hours) number of periods, with each time period potentially having a different basal rate as determined based on the historical therapy data provided by a glucose level control system 510.

Further, the table 1310 may display how the glucose level control system 510 has changed individual pump settings based on the LAI dose determination(s) displayed in table 1302. For example, the table 1310 may indicate how the usual mealtime doses have changed from a baseline or from prior pump settings by showing a change in units of insulin. Likewise, the table 1310 may indicate how the basal rates of insulin provided by the pump have changed from a baseline or from prior pump settings by showing a change in units of insulin over time.

The table 1310 may include a pump settings modification option 1312. The pump settings modification option 1312 may allow a user to modify the pump settings displayed in table 1310. Advantageously, a glucose level control system 510 that provides a pump settings modification option 1312 may allow personalized tailoring of the glucose level control system's 510 pump settings to the needs of particular subjects 512.

For example, the glucose level control system 510 may determine that one or more doses of LAI have been provided to a subject 512. The glucose level control system may display the LAI dose determination(s) in table 1302 and display modified pump settings in table 1310. In some cases, the pump settings generated by the glucose level control system 510 may be sufficient for the subject's 512 needs. However, in some instances, the pump settings displayed in table 1310 may be interpreted as a baseline recommendation for FAI glucose control therapy, and a user (e.g., a physician or other healthcare provider) may review the pump settings and use the pump settings modification option 1312 to tailor the change in dosage of insulin provided to the subject 512 to the particular subject's 512 glucose control therapy needs if such needs are different than the baseline recommendation.

### Reminder to Administer Long-Acting Insulin

FIG. 14 illustrates an example reminder to administer LAI interface 1400 in accordance with certain embodiments. The interface 1400 may display an alert in alert textbox 1402 when the glucose level control system 510 determines that the subject 512 has missed a dose of LAI. Further, the interface 1400 may display in table 1404 expected and detected doses of LAI provided to the subject 512. In some cases, the interface 1400 may be interactive and enable a user to acknowledge the alert and indicate that a dose of LAI will be administered to the subject 512, edit the alert and indicate that a dose of LAI was administered to the subject 512, or dismiss or ignore the alert.

The interface 1400 may include an alert textbox 1402. The alert textbox 1402 may display an alert generated using the process 1000, as described above with respect to FIG. 10. The alert textbox 1402 may be accompanied or replaced by one or more sounds (e.g., a chime) or visual alert (e.g., flashing lights) to attract the attention of a user. Further, the alert textbox 1402 may be formatted to attract the attention of the user (e.g., bolding, italics, enlarged text, colored text, etc.).

The alert textbox 1402 may be customized by a user. For example, the glucose level control system 510 may provide a setting whereby the user may choose one or more alert methods (e.g., sounds, visual alert, formatting, etc.) to use when the glucose level control system displays the alert textbox 1402. Further, the glucose level control system 510 may provide for a setting to customize the language of the alert. For example, a healthcare provider's contact information may be included in the alert textbox 1402.

The table 1404 of interface 1400 provides an example of a LAI dose display. The table 1404 may display an amount of LAI that the glucose level control system 510 expected would be provided to the subject 512. The expected amount of LAI may be set automatically (e.g., a LAI dose recommendation generated by the glucose level control system 510) or manually (e.g., by entry from a healthcare provider). Further, the table 1404 may display LAI doses that the glucose level control system 510 has determined were provided to the subject 512. The determined dose of LAI provided to the subject 512 may be automatically determined (e.g., a LAI dose determination generated by the glucose level control system 510) or manually entered by a user. The table 1404 may display the source of its determination that LAI was provided to the user (e.g., a glucose level sensor 516). The table 1404 may further display the difference between the expected amount of LAI provided and the LAI the glucose level control system 510 has determined was administered.

Further, the interface 1400 may include UI elements (e.g., buttons or icons, etc.) 1406, 1408, and 1410. Such UI elements may allow the user to interact with the interface 1400. For example, UI element 1406 may allow the user to indicate that the user acknowledges the alert and has or will administer a dose of LAI corresponding to the difference between the expected LAI and detected LAI. The UI element 1408 may allow the user to indicate that the user has or will administer a LAI dose that differs from the difference between the expected LAI and detected LAI and allow the user to input the different LAI dose (e.g., via a number pad). Further, the UI element 1410 may allow the user to indicate that the user has decided to dismiss or ignore the alert. Further UI elements may include a confirmation interface asking the user to confirm the user's selection. The user's indication via the UI elements 1406, 1408, and 1410 may be recorded in a log and/or transmitted to another electronic device that may be accessible by another user (e.g., a clinician or other healthcare provider, a parent or guardian, or other authorized user).

### ADDITIONAL EMBODIMENTS

Type 2 diabetes has specific characteristics such as higher insulin needs, and the ability secrete endogenous glucagon which provide opportunities to customize glucose control for subjects who suffer from Type 2 diabetes.

In some embodiments, backup therapy calculations can be leveraged for bolus and basal medicament administration. The glucose level control system can initially operate to provide a subject with all of the subject's insulin needs (e.g., using fast-acting insulin). The glucose level controller may then instruct or recommend that a user give at least some of the basal insulin portion as long-acting insulin (e.g., ⅔ or ½ of basal insulin). If daily basal (or longer-duration basal that is matched to the time action of the longer-acting insulin used) is 70 units, the glucose level controller may recommend giving 35 units of long-acting (e.g., Lantus—1 day insulin or Traciba—3 day insulin) as an LAI dose. By providing only a portion of the daily basal insulin as LAI, a buffer remains so that some basal can be given by the glucose level control system. This allows the glucose level control system to shut off or not provide at least a portion of the basal. The ratio of LAI to FAI medicament can depend on the type of long-acting insulin used. For example, when using Lantus, the ratio may be 75%, but when using Traciba, the ratio may be 65% or lower.

In some cases, when determining the basal rate of medicament for the purpose of determining an amount of basal insulin to replace with LAI, the glucose level control system may select a time of day (e.g., a particular hour) with the lowest basal rate and turn it into a daily Lantus dose. Further, in some implementations, the glucose level control system may begin with some insulin supplied as a long-acting insulin dose (e.g., 30% of basal) to give the glucose level control system a head start in determining a recommended portion of LAI, and may then adapt the recommendation for the long-acting insulin based on therapy data for the subject.

In some cases, a user may enter the amount of LAI (e.g., Lantus) dosed by the subject and may inform the glucose level control system of the amount administered so that it can adapt the glucose therapy based on the administered LAI. The user interface of the glucose level control system enables the user to enter how much insulin the subject is receiving. In some cases, when the user specifies an LAI (e.g., Lantus) dose, the glucose level control system may not use the LAI dose to modify the algorithm. In some cases, the LAI dose may be about 75% of the overall dose. The LAI dose in some such cases may not modify the operation of the basal controller. It may effectively increase insulin sensitivity. In some embodiments, the basal controller of the glucose level control system may operate normally (e.g., with the assumption that no LAI has been administered), and the basal deliveries may be compared to the LAI dose so that changes to the basal dose can be recommended based on the administered LAI dose. If the glucose level control system gives 50% of the overall insulin dose using FAI, the glucose level control system can recommend that the user revise the LAI dose to bring the ratio of LAI to medicament pump basal (e.g., FAI basal) back to 75% of the overall insulin.

If a subject uses less than 230 units of insulin per day, it is practical to use the glucose level control system to maintain the subject's diabetes. In such cases, LAI may be omitted or reduced to a nominal amount. In some cases, the glucose level control system may be configured to maintain a particular ratio between basal rate provided by LAI (e.g., by making an LAI recommendation to maintain the particular ratio) and basal rate delivered by the medicament pump. In some cases, an additional constraint on cartridge utilization can be used to determine the ratio of LAI to medicament pump or FAI usage. For example, if it is desired that each medicament cartridge lasts 2 days, a ratio of LAI to FAI may be determined to ensure that the medicament cartridge lasts 2 days or at least 2 days.

In some cases, the ratio of LAI to FAI can be selected by a user or by the control algorithm of the glucose level control system. In some cases, autonomous testing of different LAI and FAI ratios can be performed to determine the optimal LAI to FAI ratio. The testing techniques may include one or more of the techniques disclosed in the Controller Disclosures previously incorporated and/or disclosed in U.S. Pat. No. 10,940,267, the entire contents of which is hereby incorporated by reference herein for all purposes. In some cases, the glucose control system can iteratively compare different LAI/FAI ratios over time and determine the optimal ratio based on a desired glycemic outcome and/or cartridge use rate.

In some cases, the control algorithm can account for long-acting insulin supplied by a user. The glucose level control system can examine a ratio between LAI and basal delivered by the medicament pump and create one or more notifications based on the determined ratio. For example, if the ratio falls below a threshold, the glucose level control system can remind the user to give an LAI dose or to modify an LAI dose.

In some cases, the glucose level control system can start with a user-supplied basal rate and adjust it up or down within a narrow range. If the user enters the amount of LAI dosed, the glucose level control system can subtract the LAI dose from the user-supplied basal rate. Alternatively, the glucose level control system can autonomously adjust the user-supplied basal rates using autonomous learning.

In many cases, type 2 diabetics can generate endogenous glucagon, which gives a safety margin that generally isn't present for type 1 diabetics. This ability to generate endogenous glucagon can be accounted for by using a lower setpoint (e.g., 110 or 100 mg/dL) compared to the upper setpoint value used by type 1 diabetics.

In some cases, after a first period of time used to generate an LAI dose recommendation, the glucose level control system may recommend an LAI dose that may cover all insulin requirements, thereby reducing or eliminating the need for further FAI dosing.

In some cases, a subject can use backup therapy recommendations for a period of time, such as several months (e.g., 6 months). The backup therapy recommendations may be used to supply LAI and thus, the subject may not need a medicament pump or an automated glucose level control system. In some cases, pen therapy can be used to administer the LAI dose once a day. The subject can switch back to using a glucose level control system as needed (e.g., to update the LAI recommendation). Backup therapy can be used on a longer term for a number of reasons including, but not limited to, when the subject is waiting for a new pump.

In some cases, the glucose level control system can track both FAI on board and LAI on board, and use both to inform of configure the control algorithm.

In some implementations, the glucose level control system can determine if the subject forgets to inject long-acting insulin by: 1) a GUI reminder based on a last or prior UI entry and/or 2) by comparing basal insulin to a basal insulin delivery profile (e.g., by determining that a current basal rate is much higher than a prior basal rate). In some cases, a subject may take medicaments other than insulin to help control diabetes, such as metformin or Farxiga (e.g., Dapagliflozin). In such cases, the glucose level control system may be configured to determine how the additional medicaments can affect the FAI delivered over time and/or the LAI recommendation. For example, the techniques disclosed in the Controller Disclosures and/or U.S. Pat. No. 10,940,267 can be used to determine the effect of different medicament deliveries on the FAI and/or LAI dosing.

In some cases, a user interface can allow a user to select a T1D mode or T2D mode. If a T2D mode is selected, target setpoint values may be set lower and the control algorithm may be more aggressive, and initial control parameters (e.g., insulin/kg) can also be more aggressive. This UI feature may be controlled and/or safeguarded using feature enable or safe access level concepts, such as those described in U.S. Provisional Application No. 63/169,112, the entire disclosure of which is hereby incorporated by reference herein for all purposes. It may be desirable to limit the selection or modification of operating modes using the techniques described in U.S. Provisional Application No. 63/169,112 because it can be dangerous to select a T2D operating mode if the subject has T1D due, for example, to differences in setpoint range and aggressiveness in treatment.

In cases where a medicament cartridge is empty or low, the glucose level control system can provide recommendations or alerts to use fast-acting and/or long-acting insulin. These recommendations can be determined using a backup therapy algorithm, such as those described above and in U.S. Pat. No. 10,940,267.

In some cases, the user interface of the glucose level control system may permit a user to select between an “LAI mode” where a user may be administering LAI and a “no LAI mode” where a user does not administer LAI.

When the T2D operating mode is selected, a correction factor and aggressiveness factor can be higher while a setpoint can be lower. For T2D, glucose level effects may be nonlinear. Beta cells may get intoxicated by high glucose levels (e.g., 300+), and the correction requirements may be higher. Longer acting insulin allows subjects to stay locked or within a setpoint range for a longer time.

In some cases, subjects may be encouraged to use daily long-acting basal insulin delivered separately from the glucose level control system. In some such cases, the glucose level control system can still calculate and deliver additional basal insulin as needed. The amount of additional insulin delivered can be used to estimate an additional recommended amount of long-acting insulin. For example, the recommendation may be the average additional basal insulin, 24 hours times the minimum hourly additional basal insulin or some other function.

The glucose level control system may similarly determine that the basal insulin is too high and recommend a reduction in long-acting insulin. In some cases, the glucose level control system may allow UI entry of long-acting insulin and add that into the display of TDD of insulin. It is also possible for the glucose level control system to breakout or separate from the TDD the amount of insulin attributable to the long-acting insulin dose, autonomous basal, meal insulin, and/or correction insulin enabling a user to determine the constituent parts of the TDD of insulin.

Glucose Level Control System with Non-Automated Dosing Overview

In some cases, a user may manage a subject's disease by using a glucose level control system. The glucose level control system may be used to control an ambulatory medicament device (e.g., an insulin infusion pump). The ambulatory medicament device may use a control algorithm to help determine a quantity of medicament to deliver to a subject and under what circumstances to deliver the medicament. In some cases, the control algorithm may adapt over time based at least in part on a subject's past therapy history and/or glucose levels. Adapting the control algorithm over time may help improve maintenance of the subject's disease. In some cases, the control algorithm adjusts the deliveries to maintain a subject's glucose levels within a desired range (e.g., between 60 mg/dL and 170 mg/dL).

In some cases, a user may use injection therapy using a needle or a medicament pen (e.g., a smart pen) instead of the ambulatory medicament device to manage the subject's disease. For example, some users may not have access to an ambulatory medicament device (e.g., due to insurance coverage, cost, damage or malfunction of the subject's ambulatory medicament device, etc.). In some cases, a user may want to alternate between using the ambulatory medicament device and alternative treatment tools, such as injection therapy (e.g., due to costs, types of available insulin, insulin usage rate, etc.). Advantageously, in some embodiments, the glucose level control system may be operable without the ambulatory medicament device. For example, the glucose level control system can use the control algorithm to determine a recommended medicament dose for the subject, and a user may manually administer the recommended medicament dose or may use a medicament delivery device to manually administer the medicament dose, such as an insulin pen or smart pen.

In some cases, the glucose level control system outputs an indication of the recommended medicament dose to the user. For instance, the glucose level control system may output the indication of the recommended medicament dose on a display of the glucose level control system. Alternatively, or in addition, the glucose level control system may transmit the indication of the recommended medicament dose to an electronic device, such as a smart pen. In some cases, the user can administer medicament to the subject through injection therapy or a smart pen based on the indication of the recommended medicament dose. Thus, a user can use the glucose level control system together with injection therapy or with a smart pen, regardless of whether the subject has access to an ambulatory medicament device that automatically administers medicament.

The glucose level control system can include a controller configured to generate dose control signals for one or more glucose level control agents that can be infused into the subject. Alternatively, or in addition, the controller can be configured to determine a recommended medicament dose of one or more glucose level control agents. In some cases, the controller can be configured to transmit an indication of the recommended medicament dose of one or more glucose level control agents. Glucose level control agents may include regulatory agents, such as insulin and insulin analogs, that can decrease glucose levels in the subject and counter-regulatory agents, such as glucagon or dextrose, that can increase glucose levels in the subject. Glucose level control agents can be delivered to a subject via subcutaneous injection, via intravenous injection, or via another suitable delivery method.

In some case, the glucose level control system may be configured (e.g., via dose control signals) to cause a delivery device 514 to administer one or more glucose level control agents. Alternatively, or in addition, the glucose level control system may be configured to output a recommendation of one or more doses of one or more glucose level control agents enabling a user to administer the one or more doses via a manual delivery device, such as an insulin pen. For example, the glucose level control system can display an indication of a recommended medicament dose on a display of the glucose level control system or transmit the indication to another electronic device, such as a smart pen.

In some embodiments, a glucose level control system can generate a dose control signal for a medicament regardless of whether the medicament is available for dosing via a medicament pump operatively connected to the subject. For example, the glucose level control system can transmit the dose control signal to an electronic device, such as a smart pen, via a wireless connection. The dose control signal can be used to preconfigure the smart pen based on the recommended dose of the medicament. Advantageously, preconfiguring the smart pen based on the recommended dose of the medicament enables a user to administer the medicament dose with a simplified user interface (e.g., a click of a button) and/or without manually configuring the smart pen. Additionally, some embodiments disclosed herein relate to a glucose level control system capable of determining whether long-acting insulin has been provided to a subject either via the smart pen or through another delivery method. Further, the system may modify the fast-acting insulin therapy provided to the subject in response to determining that long-acting insulin has or has not been administered to the subject.

### Example Glucose Level Control System

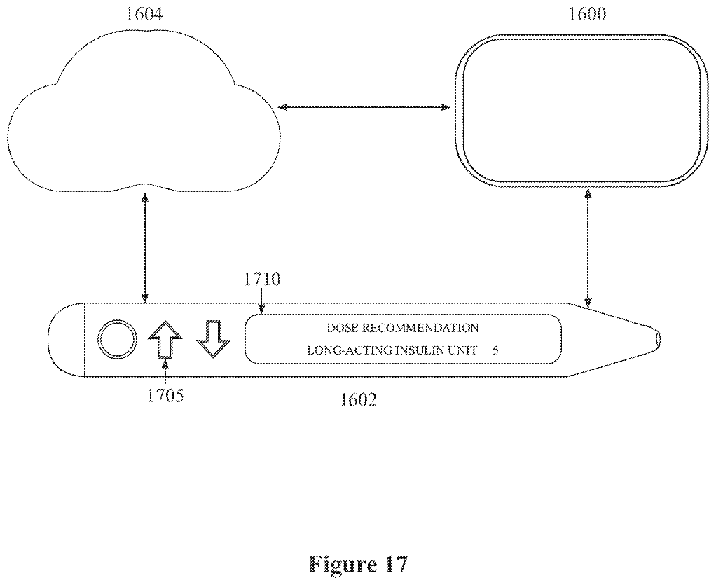

FIGS. 16 and 17 illustrate an automated glucose level control system 1600 that can regulate the glucose level of a subject. In some embodiments, the glucose level control system 1600 may output an indication of a recommended medicament dose to a display of the glucose level control system 1600 or to another electronic device (e.g., a smart pen 1602). The indication may be communicated directly or via a network 1604 to the electronic device (e.g., the smart pen 1602). A user may receive an indication of a recommended dose of medicament from the glucose level control system 1600 and use the smart pen 1602 to administer the recommended medicament dose. The indication may be received via a user interface of the glucose level control system 1600 (e.g., via the user interface circuitry 534), a user interface of the smart pen 1602, or via a user interface of another electronic device (e.g., a smartphone, a smartwatch, a tablet, etc.). It should be noted that the glucose level control system 1600 is an example of a medicament infusion system and may include one or more of the embodiments previously described above with respect to medicament infusion systems (e.g., glucose level control system 510).

In some embodiments, the glucose level control system 1600 may have an online mode or a closed-loop mode. In the closed-loop mode, the glucose level control system 1600 may receive, via the sensor interface 1606, one or more glucose level signals from one or more glucose level sensors 516 (e.g., a CGM sensor). Further, the glucose level control system 1600 can receive information about the subject from other sensors, such as a heartrate sensor, a blood oxygen sensor, and/or a blood pressure sensor. In some embodiments, the one or more sensors are wearables that are operatively connected to a subject's body (e.g., a smartwatch or a patch). Additionally, in some embodiments, the glucose level control system 1600 determines a recommended dose of medicament based at least in part on the subject's physiological data, which may be received from the one or more sensors (e.g., the glucose level sensor 516).

In some embodiments, the glucose level control system 1600 is able to operate in an offline mode or an open-loop mode. In the open-loop mode, the glucose level control system 1600 may provide delivery of medicament, independent of or without receipt of the subject's physiological data (e.g., glucose level) reported by a sensor (e.g., glucose level sensor 516). For example, in cases where one or more sensors need replacing, are not properly connected to the subject, or are defective, the glucose level control system 1600 may operate in an open-loop manner without input from the one or more sensors. Alternatively, or in addition, in the open-loop mode, the glucose level control system 1600 may provide delivery of medicament, independent of or without receipt of an indication of insulin administered or reportedly administered to the subject. In some cases, the glucose level control system 1600 may use a therapy history and/or a history of prior glucose level control measurements to facilitate automatic operation of the glucose level control system 1600 for at least a particular time period. Thus, operation may be divided between closed-loop periods each including a succession of sampling intervals when, for example, a glucose signal (e.g., glucose level, see FIG. 6) and/or one or more indications of administered or reportedly administered insulin is available, and open-loop periods each including a succession of sampling intervals when the glucose signal and/or one or more indications of administered or reportedly administered insulin are either completely or intermittently unavailable. The description below uses the terms “closed-loop” and “open-loop” for periods of time with and without feedback from a glucose level sensor and/or one or more indications of administered or reportedly administered insulin.

As described above, the glucose level control system 1600 can have one or more controllers 518, a processor 530, an input/output circuitry 532, and a memory 540. In some embodiments, the one or more controllers 518 may generate or determine a recommended dose of medicament. Alternatively, or in addition, the processor 530 may determine a recommended dose of medicament. The input/output circuitry 532 may output an indication of the recommended dose of medicament onto a display. Alternatively, or in addition, the input/output circuitry 532 may transmit the indication of the recommended dose of medicament to a smart pen 1602 or other electronic device (e.g., a smartphone, a patch pump, or smartglasses, etc.). In some embodiments, the network interface circuitry 536 enables communication with a network 1604.

In some embodiments, with reference to FIG. 17, a display 1710 of the smart pen 1602 can display the indication of the recommended medicament dose received from the glucose level control system 1600. Additionally, or alternatively, the display 1710 can display an indication of a recommended medicament dose received via the network 1604 (e.g., a recommended medicament dose provided by a healthcare provider). In some embodiments, the indication of the recommended medicament dose can be accessed by other electronic devices (e.g., smartphone, laptop, or smartwatch) that can communicate with the network 1604. Furthermore, the input/output circuitry 532 can provide interface(s) to electronic devices such as a glucose level sensor 516 and the smart pen 1602.

FIG. 17 illustrates an example glucose level control system environment that provides glucose control via a smart pen 1602 in accordance with certain embodiments. The glucose level control system environment may have a glucose level control system 1600, a smart pen 1602, and a network 1604. The smart pen 1602 may have a processor, memory, a power source (e.g., batteries), and a network interface circuitry. In some embodiments, the smart pen 1602 can communicate with the glucose level control system 1600 and a network 1604. For instance, the smart pen 1602 may have a transceiver to communicate to the network interface circuitry 536 of the glucose level control system 1600 using Bluetooth, Bluetooth Low Energy (BLE), 4g LTE, 5G, ND-LTE, or another wireless protocol.

While the illustrated embodiments of the smart pen 1602 has three control buttons 1705 and a display 1710, it should be understood that the smart pen 1602 may have more or fewer buttons. Further, while described as control buttons, it should be understood that the control buttons 1705 may be any type of user interface element. For instance, the smart pen 1602 can have one to five control buttons 1705, a touchscreen, a dial or knob, etc. The control buttons 1705 can configure the medicament dose to be administered. For example, a user may change the type of medicament (e.g., from long-acting insulin to glucagon) or the size of medicament (e.g., from 8 units of insulin to 10 units of insulin) using one or more control buttons 1705. Additionally, the control buttons 1705 can be used to review a log of the doses that have been recommended for or administered to the subject. For example, a user can view the timing and/or quantity of the last administered dose on the display 1710 by interacting with a control button 1705. In some embodiments, the smart pen 1602 is controlled via an external user interface. For example, a user may configure the type or size of medicament using an external electronic device (e.g., the glucose level control system 1600, smartphone, laptop, or smartwatch). Furthermore, in some embodiments, a user can review the smart pen's 1602 log of recommended or administered doses via an external electronic device (e.g., through an application on a smartphone).

In some embodiments, the smart pen 1602 can have a hypodermic needle, and one or more replaceable cartridges. The hypodermic needle can be a retractable needle. The needle can be spring-loaded and/or disposable. In some embodiments, the disposable hypodermic needle uses leak-free connectors (e.g., a Luer taper connector) to attach and detach from the smart pen 1602. Additionally, or alternatively, the hypodermic needle may have a protective cap (e.g., the cap must be removed before an injection can be administered). In some embodiments, the smart pen 1602 is disposable and designed for a single injection (e.g., an autoinjector).

In some embodiments, the smart pen 1602 may deliver doses of insulin to a user (e.g., a smart insulin pen). The smart pen 1602 may have one or more replaceable cartridges for insulin. Alternatively, the smart pen 1602 can have a refillable medicament reservoir. In some embodiments, the smart pen 1602 can deliver fast-acting insulin, long-acting insulin, or another type of insulin. Furthermore, some embodiments of the smart pen 1602 can deliver multiple types of insulin (e.g., fast-acting insulin for a first recommended medicament dose, and long-acting insulin for a second recommended medicament dose). Additionally, the smart pen 1602 may optionally deliver a counter-regulatory agent. For example, the smart pen 1602 can be a bihormonal pen. Thus, in some embodiments, a user can use the smart pen 1602 to prevent or reduce hypoglycemia and/or hyperglycemia.

In some embodiments, a user may use the smart pen 1602 along or in combination with an ambulatory medicament pump. For example, a user may administer medicament doses via a smart pen 1602 to supplement the medicament doses a subject receives via the ambulatory medicament pump. For instance, a subject may receive doses of fast-acting insulin via the ambulatory medicament pump and doses of long-acting insulin via the smart pen 1602. Additionally, or alternatively, a user may use the smart pen 1602 to administer medicament doses to a subject when an ambulatory medicament pump is not available (e.g., the ambulatory medicament pump needs replacing, is not properly connected to the subject, or is defective). However, in some embodiments, a user uses only the smart pen 1602 to administer medicament doses to the subject (e.g., the subject may not have an ambulatory medicament pump).

In some embodiments, the smart pen 1602 can administer a medicament dose independently of the glucose level control system 1600. For example, a user can configure and administer a dose of medicament via the smart pen 1602 regardless of whether the smart pen 1602 receives a communication from the glucose level control system 1600. However, in other embodiments, the smart pen 1602 may be configured to only permit administration of medicament upon receipt of an indication of a recommended medicament dose from the glucose level control system 1600. For example, the smart pen 1602 may have a safety lock that prevents the user (e.g., a child) from manually configuring the smart pen 1602. The safety lock may help prevent users with diminished faculties from configuring the smart pen 1602 with an incorrect dose of medicament (e.g., a dose of medicament that is not consistent with the subject's treatment plan). Alternatively, or in addition, the safety lock may prevent users who are children from configuring the smart pen 1602 with an incorrect dose of medicament. Examples of safety locks that may be implemented by the smart pen 1602 and/or a glucose level control system 1600 in conjunction with one or more of the embodiments disclosed herein are disclosed in U.S. Pat. No. 11,135,365, which is hereby incorporated by reference herein in its entirety and for all purposes.

Additionally, the smart pen 1602 can be used while the glucose level control system 1600 is operating in either closed-loop mode or open-loop mode. For example, when operating in closed-loop mode, the glucose level control system 1600 can modify a recommended medicament dose based at least in part on a glucose level of the subject and administered or reportedly administered insulin. The modified recommended medicament dose may be transmitted to the smart pen 1602. As another example, when operating in open-loop mode, the glucose level control system 1600 may transmit an indication of a recommended medicament dose to the smart pen 1602 regardless of whether the one or more sensors of the glucose level control system 1600 are functioning or connected (e.g., a test strip may be used to obtain the glucose level of the subject).

### Medicament Dose Recommendation Process

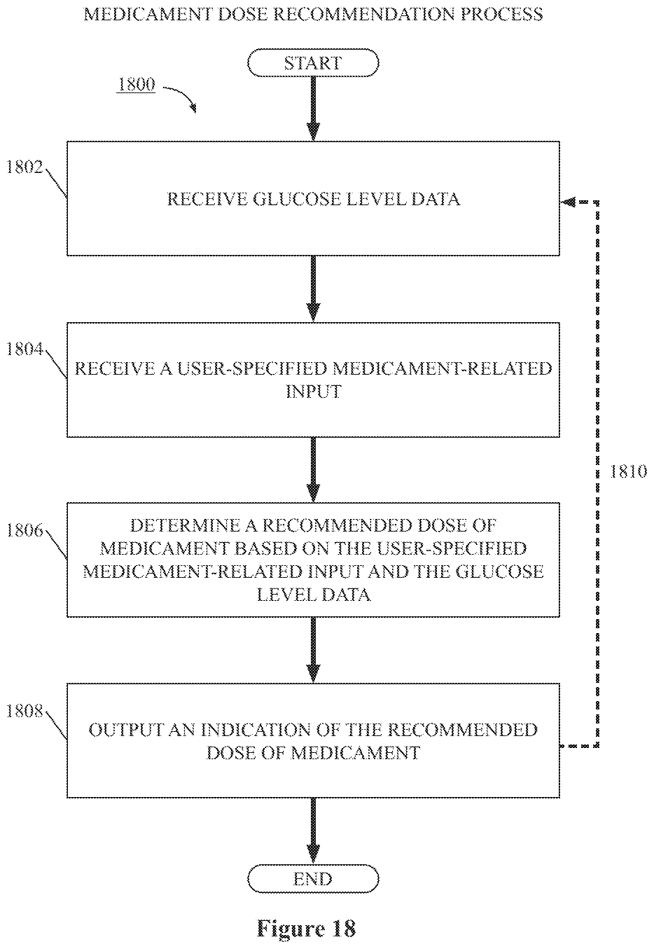

FIG. 18 presents a flowchart of an example medicament dose recommendation process 1800 in accordance with certain embodiments. The process 1800 may be performed by any system that can generate a medicament dose recommendation for controlling the glucose level of a subject. For example, the process 1800 may be performed by the glucose level control system 1600, an ambulatory medicament system, an ambulatory medical device, or an ambulatory medical pump. In some embodiments, the process 1800 may be at least partly performed by one or more elements of the glucose level control system 1600, such as the processor 530, one or more controllers 518, the memory 540, or an input/output circuitry 532. In some cases, at least certain operations of the process 1800 may be performed by a separate computing system that receives therapy data corresponding to the subject. In some embodiments, the therapy data may include the subject's glucose level data obtained from the glucose level sensor or from glucose test strips. Alternatively, or in addition, the therapy data may correspond to glucose control therapy provided to the subject. Although one or more different systems may perform one or more operations of the process 1800, to simplify discussions and not to limit the present disclosure, the process 1800 is described with respect to particular systems.

The process 1800 begins at block 1802 where, for example, the glucose level control system 1600 receives glucose level data associated with the glucose level of the subject. The glucose level control system 1600 may receive the glucose level data from a glucose sensor that is operatively connected to the subject. In some embodiments, the glucose sensor may be skin mountable. For example, the glucose sensor may have an adhesive patch that attaches to a subject's arm or abdomen. The glucose sensor may have a subcutaneous cannula to continuously measure the subject's glucose level. In some embodiments, the glucose sensor may measure glucose levels continuously or periodically (e.g., one reading per minute, per five minutes, per hour, etc.). In some cases, the glucose level data may be transmitted wirelessly from the glucose sensor to the sensor interface 1606 of the glucose level control system 1600.

In some cases, glucose level data may be determined from one or more isolated glucose measurements. The isolated glucose measurement may be in addition to or as a substitute for measurements obtained from a CGM sensor. For instance, the glucose level data may be obtained from non-automated and/or user administered tests. For example, some users may use glucose level test strips and a strip meter to measure the subject's glucose level. In some embodiments, a user can enter the isolated glucose measurements via the user interface of the glucose level control system 1600. However, in other embodiments, a strip test meter is built into the glucose level control system 1600. Thus, the glucose level control system 1600 may automatically store the results of the glucose level test strips.

At block 1804, the glucose level control system 1600 receives a user-specified medicament-related input. The user-specified medicament-related input may include a request for a dose of medicament to be administered and/or a request for a recommended dose of medicament. The user-specified medicament-related input may identify a quantitative amount of medicament. Alternatively, or in addition, the user-specified medicament-related input may include a quantitative measurement that directly or indirectly corresponds to a medicament dose. For example, the user-specified medicament dose may correspond to a measurement of food to be consumed. In some cases, the user-specified medicament-related input corresponds to a medicament dose type and/or size. However, in other cases, the user-specified medicament-related input may be metadata that can be used to determine or recommend a medicament dose of a particular size or type.

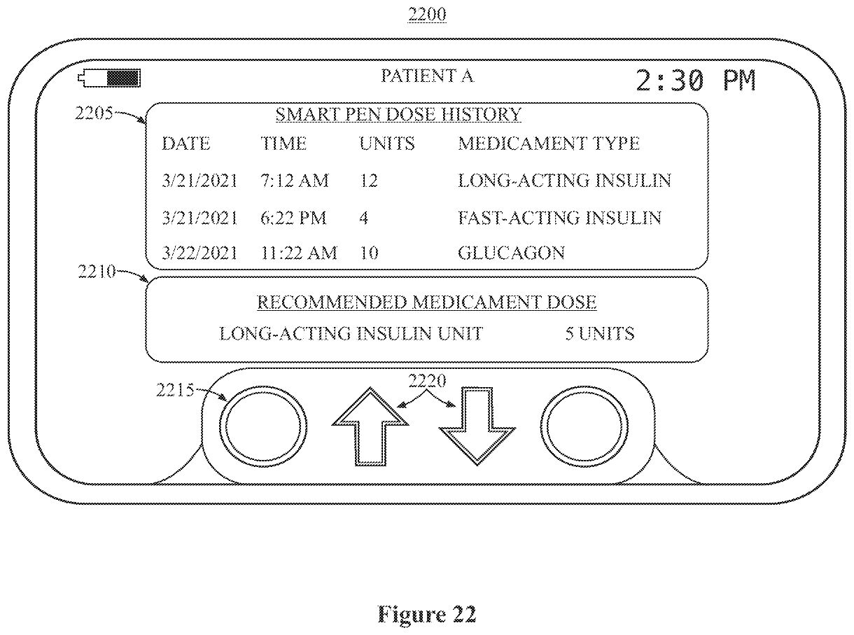

In some embodiments, the glucose level control system 1600 receives the user-specified medicament-related input via user interaction with a dose setup interface. The dose setup interface may be a graphical user interface (GUI) that may be displayed on a display. One non-limiting example user interface is illustrated in FIG. 22 and described in more detail below. In some embodiments, the dose setup interface enables the user to enter the type and quantity of medicament that the user intends to administer to the subject. More specifically, the user can input a quantitative amount of medicament into the user interface. The quantitative amount of medicament can be specified by a volume of medicament (e.g., 0.3 ml, 0.5 ml, etc.), a number of units of medicament (e.g., 5 units, 10 units, etc.), or any other measurement scale. Additionally, the user-specified quantitative dose of medicament can be fast-acting insulin, long-acting insulin, a counter-regulatory agent, or any other type of medicament that may be used to manage the subject's disease. For example, the user can input a medicament dose of “0.5 ml of long-acting insulin” or “10 units of glucagon.”

In some cases, the user-specified medicament-related input can be a quantitative characterization or a quantitative indication of one or more characteristics of food to be consumed, such as one or more macronutrients. For instance, a user can input macronutrients (e.g., 44 g of carbohydrates, 33 g of fat, and 26 g of protein) associated with food that the subject is consuming or will consume into the glucose level control system 1600. Alternatively, a user can input the number of carbohydrates (e.g., 44 g of carbohydrates) the subject is consuming or will consume into the glucose level control system 1600. In some embodiments, the glucose level control system 1600 may determine a recommended amount of medicament based at least in part on the identified macronutrients of food consumed or to be consumed.

In some embodiments, the user-specified medicament-related input can be a qualitative amount of food. For example, a user can input a meal announcement. The meal announcement can include the meal size of a meal or food the subject has consumed or intends to consume. The user can input the meal announcement into the user interface of the glucose level control system 1600 or an electronic device in communication with the glucose level control system 1600. More specifically, a user can input that the subject has consumed or plans to consume a small, medium, or large meal. It should be understood that meal or food-intake may not be limited to defined meals, but may include food-intake generally, such as snacks. In some cases, the food-intake is associated with the consumption of food of a minimum size or macronutrient level. In other cases, the food-intake may include any amount of food. In some embodiments, the glucose level control system 1600 may determine a recommended amount of medicament based at least in part on the identified meal size of a meal or identified food-intake. In some embodiments, the glucose level control system 1600 can receive both qualitative and quantitative medicament-related inputs (e.g., a meal-size and a size of a medicament dose).

In some embodiments, the user-specified medicament-related input may include physiological data (e.g., weight, gender, or age, etc.) of the subject. In some embodiments, block 1802 is optional or omitted. For example, the glucose level control system 1600 may determine a recommended dose of medicament based at least in part on the subject's physiological data and/or a subject's food intake without considering glucose level data. For instance, a user can enter the weight (e.g., 65 kg) of the subject and the meal size of a meal (e.g., a medium meal, or a medium-sized lunch meal) to be consumed by the subject into a user interface of or in communication with the glucose level control system 1600. Based on the input food-intake data, the glucose level control system 1600 can recommend a medicament dose (e.g., 5 units of long-acting insulin). Alternatively, the glucose level control system 1600 can use a subject's glucose level data and one or more user-specified medicament-related inputs (e.g., the subject's weight and food-intake) to determine a recommended dose of medicament for the subject.

At block 1806, the glucose level control system 1600 determines a recommended dose of medicament based on the user-specified medicament-related input and the subject's glucose level data. In some embodiments, the processor 530 of the glucose level control system 1600 uses a control algorithm to determine a recommended dose of medicament for the subject. Additionally, the control algorithm may be configured to control the glucose levels of a subject (e.g., by determining and outputting indications of recommended medicament doses). As described above, the control algorithm can generate at least one of a recommended basal dose, a recommended correction dose, and/or a recommended meal dose. The recommended correction dose can include regulatory agents, such as fast-acting insulin and long-acting insulin and can be generated using a model-predictive control (MPC) algorithm such as the MPC algorithms disclosed in the Controller Disclosures. The recommended basal dose can include a regulatory agent and can be generated using a basal control algorithm such as disclosed in the Controller Disclosures. Similarly, the recommended meal dose can include a regulatory agent and can be generated using a meal control algorithm such as disclosed in the Controller Disclosures.

In some embodiments, the glucose level control system 1600 may use the user-specified medicament-related input and/or the glucose level data as control parameter values or to determine control parameter values used by the control algorithm. For instance, one or more control parameters can be set based at least in part on the subject's glucose level data. For example, one or more control parameters may be set based at least in part on the subject's glucose level trends (e.g., subject's glucose level history) and/or on the subject's current glucose level. Alternatively, or in addition, one or more control parameters can be based at least in part on the user-specified medicament-related input. For example, one or more control parameters may be set based at least in part on a user-specified amount of medicament (e.g., an amount of medicament the user intends to administer to the subject) and/or on a user-specified amount of food-intake (e.g., the meal size of a meal the subject has consumed or plans on consuming). Similarly, one or more control parameters may be set based at least in part on user-specified physiological data (e.g., weight, age, or gender of the subject) or on any other user-specified medicament-related input.

In some embodiments, the glucose level control system 1600 may determine a recommended medicament dose based on one or more control parameters. For example, the glucose level control system 1600 can use a history of the subject's glucose level data to determine the size of the recommended medicament dose. For instance, the glucose level control system 1600 may determine a smaller recommended medicament dose (e.g., four units of insulin instead of five units) if the subject's glucose levels are often low (e.g., due to insulin absorption rate or poor food-intake macronutrient estimates). Alternatively, or in addition, the glucose level control system 1600 may determine a larger recommended medicament dose (e.g., six units of insulin instead of five) if the subject's glucose levels are often high.

In some embodiments, the glucose level control system 1600 determines the recommended medicament dose based at least in part on the subject's glucose level trends. For example, if a subject's glucose level has been rising for a certain time period (e.g., the previous one to twelve hours), the glucose level control system 1600 may determine a larger recommended medicament dose as compared to a recommended medicament dose for a subject without rising glucose levels or with a relatively steady glucose level. Alternatively, if a subject's glucose levels have been steady (e.g., glucose levels have had limited total upward or downward movement, such as less than plus or minus 20 mg/dL) for a certain time period (e.g., the previous one to twelve hours), the glucose level control system 1600 may determine a smaller recommended medicament dose as compared to a recommended medicament dose for a subject with upward trending glucose levels.

In some embodiments, the glucose level control system 1600 determines a recommended dose of medicament based at least partly on the subject's glucose history. For example, if a subject's glucose levels consistently increase during a certain time of day (e.g., a mealtime), the glucose level control system 1600 may predict that the subject consumes a meal at that time of day and may generate a recommendation of a dose of medicament based at least in part on the predicted food-intake consumption. In some embodiments, the glucose level control system 1600 can determine a recommended medicament dose of zero units of insulin. For example, if a subject's glucose level is low, the glucose level control system 1600 can recommend a dose of zero insulin units. In some embodiments, the glucose level control system 1600 may recommend a dose of a counter-regulatory agent if the subject's glucose level is very low (e.g., subject is experiencing or is close to experiencing a hypoglycemic episode). Alternatively, or in addition, the glucose level control system 1600 may determine a recommended medicament dose of a counter-regulatory agent if a subject's glucose levels are trending downward towards a hypoglycemic range (e.g., below 70 mg/dL).

In some embodiments, the glucose level control system 1600 determines the recommended dose of medicament based at least in part on the subject's insulin sensitivity (e.g., how quickly and/or how much a subject's glucose level responds to a particular dose of insulin). In some embodiments, the user-specified medicament-related input is the subject's insulin sensitivity. Alternatively, the glucose level control system 1600 can determine a subject's insulin sensitivity and adjust one or more control parameters accordingly. For example, if the subject's insulin sensitivity is low (e.g., a subject's glucose levels respond less to insulin compared to an average similar person in the general population), the glucose level control system 1600 may determine a larger recommended dose of medicament as compared to a recommended medicament dose for a subject with a higher insulin sensitivity. Alternatively, if the subject's insulin sensitivity is high (e.g., a subject's glucose levels respond more to insulin compared to an average similar person in the general population), the glucose level control system 1600 may determine a smaller recommended dose of medicament than it would have otherwise. In some embodiments, the glucose level control system 1600 determines the subject's insulin sensitivity by analyzing a subject's past therapy history (e.g., administered medicament doses) and/or glucose levels.

In some embodiments, the glucose level control system 1600 can generate a recommended dose of medicament while the glucose level control system 1600 is operating in open-loop mode. In open-loop mode, the glucose level control system 1600 may use isolated glucose measurements (such as, for example, manual measurements obtained from glucose test strips) as control parameter values or to determine control parameter values. Alternatively, the glucose level control system 1600 can generate a recommended dose of medicament without glucose level data (e.g., the recommended medicament dose may be at least partly based on one or more user-specified medicament-related inputs, such as the subject's food-intake and/or weight, etc.).

In some embodiments, the glucose level control system 1600 determines the recommended dose of medicament based at least in part on the user-specified medicament-related input. For example, the glucose level control system 1600 may use a user-specified dose of medicament (e.g., the type and amount of medicament that a user intends to administer to the subject) as a baseline for the recommended medicament dose. The glucose level control system 1600 can determine a recommended medicament dose that is larger or smaller than the user-specified dose of medicament based on one or more control parameters. In some embodiments, the glucose level control system 1600 provides a recommended medicament dose of the same type as the user-specified dose of medicament. For instance, if the user-specified dose of medicament is fast-acting insulin, the glucose level control system 1600 may determine a recommended medicament dose of fast-acting insulin.

In some cases, the user-specified medicament-related input may be a size of a medicament dose that a user or subject intends to administer to the subject. In some such cases, the glucose level control system 1600 can determine whether the user-specified medicament dose size satisfies one or more approval conditions. For example, a user (e.g., a physician or other healthcare provider) may set one or more approval conditions to prevent a subject from receiving a medicament dose that is not in accordance with the subject's treatment plan. In some embodiments, the approval conditions may require that the medicament dose is below a maximum dose size or above a minimum dose size. Alternatively, or in addition, the approval conditions may require the medicament dose be of a certain medicament type or a certain medicament concentration (e.g., U-100, U-200, etc.). In some cases, the maximum or minimum dose size may be restricted based at least in part on the medicament concentration. In some embodiments, the approval condition may be associated with a difference between a user-inputted medicament dose size and a recommended medicament dose, such as requiring the difference to be below a threshold value.

When the approval condition is a maximum difference between the size of the user-inputted medicament dose and the size of the recommended medicament dose, the glucose level control system 1600 can compare the size of the user-specified medicament dose and the size of the recommended medicament dose to determine if they match or match within a threshold value. For example, the glucose level control system 1600 can calculate the percentage difference between the size of the user-specified medicament dose and the size of the recommended medicament dose and determine whether the percentage difference is below a threshold value (e.g., 5%). If the glucose level control system 1600 determines that the difference between the user-specified medicament dose and the recommended dose of medicament is less than the threshold value, the glucose level control system 1600 can output an indication that the user-specified medicament-related input satisfies the approval condition (e.g., the threshold). Alternatively, or in addition, if the user-specified medicament dose and the recommended dose of medicament match within the threshold value, the glucose level control system 1600 can output an indication of the recommended dose of medicament. In some embodiments, if the glucose level control system 1600 determines that the user-specified medicament dose and the recommended medicament dose do not match within a threshold value, the glucose level control system 1600 can output an indication that the approval condition has not been satisfied. Alternatively, or in addition, if the glucose level control system 1600 determines that the user-specified medicament dose and the recommended medicament dose do not match within a threshold value, the glucose level control system 1600 can output an indication of a modification to the user-specified medicament-related input. The modification may be based at least in part on the difference between the recommended dose of medicament and the user-specified medicament-related input.

In some embodiments, the glucose level control system 1600 may determine a recommended medicament dose at least partly based on a user-specified value of one or more macronutrients that the subject has consumed or intends to consume. For example, if the subject's intended food-intake includes more macronutrients than the subject's past or most recent set of food-intake events, the glucose level control system 1600 may determine a larger recommended dose of medicament compared to the past or most recent set of recommended medicament doses. Alternatively, if the subject's intended food-intake includes less macronutrients than the subject's past or most recent set of food-intakes, the glucose level control system 1600 may determine a smaller recommended dose of medicament compared to the past or most recent set of recommended medicament doses.

In some embodiments, the glucose level control system 1600 may determine a recommended dose of medicament based at least in part on a meal announcement (e.g., a user-specified meal size of a meal the subject has consumed or intends to consume). A user can input that the subject has consumed or will consume a small, medium, or large meal. In some embodiments, the glucose level control system 1600 provides meal size descriptions (e.g., through a user manual or via electronic messages) to help a user select the meal size. For example, the glucose level control system 1600 may describe a small meal as a meal with less than 300 calories, a medium meal as a meal with 300-600 calories, and a large meal as a meal with more than 600 calories. Alternatively, or in addition, the glucose level control system 1600 may describe meal sizes in terms of weight (e.g., grams), volume (e.g., cups), or any other measurement scale. In some cases, the meal sizes may be based at least in part on macronutrient contents of the meal. For example, a small meal with an above average carbohydrate count may be classified as a medium meal. In some embodiments, the meal sizes are defined by a subject's past eating habits. For example, a user makes a small meal announcement for meals that are smaller than the subject's average meal, a medium meal announcement for meals that are about the same size as the subject's average meal, and a large meal announcement for meals that are larger than the subject's average meal. It should be noted that the glucose level control system 1600 may have more than three meal size options. For instance, in some embodiments, a user can select an extra-small meal or extra-large meal.

In some embodiments, the glucose level control system 1600 may determine a recommended medicament dose of the same relative size as the meal. For example, if the user specifies a small meal, the glucose level control system 1600 may determine a smaller recommended medicament dose than if the user had specified a medium meal. Similarly, if the user specifies a large meal, the glucose level control system 1600 may determine a larger recommended medicament dose than if the user had specified a medium meal. For example, the glucose level control system 1600 may determine a recommended medicament dose of 6 units of fast-acting insulin for a small meal, 8 units of fast-acting insulin for a medium meal, and 10 units of fast-acting insulin for a large meal. Alternatively, or in addition, the glucose level control system 1600 may adjust the size of the recommended medicament dose based on the meal size. For example, the glucose level control system 1600 may increase the size of the recommended medicament dose if the user specifies a large meal and decrease the size of the recommended medicament dose if the user specifies a small meal. In some embodiments, the glucose level control system 1600 may adapt the size of the recommended medicament dose provided for each meal size over time. For example, if the user specifies a meal size, and the recommended medicament dose provided does not reduce the subject's glucose levels to a desired range (e.g., the subject experiences symptoms of hyperglycemia), the size of the recommended medicament dose may be raised for future meal announcements of the same size. Similarly, if the user specifies a meal size, and the recommended medicament dose reduces the subject's glucose levels below the desired range (e.g., the subject experiences hypoglycemic symptoms), the size of the recommended medicament dose may be lowered for future meal announcements of the same size.

In some embodiments, the glucose level control system 1600 can use the subject's physiological data to determine a recommended medicament dose. For example, the glucose level control system 1600 may determine a larger recommended medicament dose if the weight of the subject is above a certain threshold (e.g., 70 kg) than if it is below that threshold. Similarly, the glucose level control system 1600 may determine a smaller recommended medicament dose if the weight of the subject is below a certain threshold (e.g., 50 kg) than if it is above that threshold. It should be understood that the glucose level control system 1600 can determine the recommended medicament dose based on multiple control parameters. For example, the glucose level control system 1600 may determine the recommended medicament dose based on the user-specified meal size, the subject's physiological data, and the subject's glucose level data. Overall, in some embodiments, the glucose level control system 1600 assists a user in managing a subject's glucose level by determining one or more recommended doses of medicament based at least in part on the user-specified medicament-related input and the subject's glucose level data.

At block 1808, the glucose level control system 1600 can output an indication of the recommended dose of medicament. For example, the glucose level control system 1600 can transmit the indication of the recommended dose of medicament to a display of the glucose level control system 1600. Alternatively, or in addition, the glucose level control system 1600 can transmit the indication of the recommended medicament dose to an electronic device such as the smart pen 1602, a smartphone, or any other type of electronic device that may communicate with the glucose level control system 1600. The smart pen 1602 can display the indication of the recommended dose of medicament (e.g., the size, type, and/or concentration of the medicament) on the display 1710 of the smart pen 1602. A user can configure the smart pen 1602 according to the recommended dose of medicament (e.g., configure the smart pen 1602 to administer the recommended medicament dose).

Alternatively, the smart pen 1602 may be automatically configured based on the recommended dose of medicament. Accordingly, if the user desires to administer the recommended dose of medicament, the user can initiate administration of the automatically configured medicament dose without manually configuring the smart pen 1602 with the recommended medicament dose. For example, the glucose level control system 1600 can send a dose control signal based on the recommended dose of medicament to the smart pen 1602. In some embodiments, the dose control signal automatically configures the smart pen 1602 according to the determined recommended medicament dose. In some cases, a user may confirm the recommended medicament dose using, for example, a user interface of the smart pen 1602. The glucose level control system 1600 may configure the smart pen 1602 to administer the recommended dose of medicament in response to the confirmation by the user of the recommendation. Thus, the glucose level control system 1600 can reduce user errors (e.g., by reducing user inputs) and streamline the medicament delivery process when using an insulin pen or smart pen 1602.

In some implementations, the block 1808 may include storing the indication of the recommended medicament dose in a memory 540 of the glucose level control system 1600 and/or in a remote storage. Alternatively, or in addition, the glucose level control system 1600 may store the indication of the recommended medicament dose on a removable memory device (e.g., a micro-Secure Digital card). In some embodiments, the glucose level control system 1600 may store the indications of prior recommended medicament doses as part of the subject's therapy history. Alternatively, or in addition, the subject's therapy history may include whether prior recommended medicament doses were administered to the subject.

In some embodiments, the glucose level control system 1600 may determine a recommended medicament dose based at least in part on one or more stored indications of previously recommended medicament doses. The glucose level control system 1600 may determine a recommended medicament dose based on the average size of the subject's prior recommended medicament doses. For example, if the average size of the previously recommended medicament doses is five units of fast-acting insulin, the glucose level control system 1600 may determine a recommended medicament dose that is about five units of fast-acting insulin. Alternatively, or in addition, the glucose level control system 1600 may determine a recommended medicament dose based at least in part on a trend of the previously recommended medicament doses. For example, if the size of the previously recommended medicament doses is trending upward (e.g., each recommended medicament dose is 5% larger than the previously recommended medicament dose), the glucose level control system 1600 may determine a recommended medicament dose that continues the trend (e.g., the recommended medicament dose may be about 5% larger than the previously recommended medicament dose). Determining a recommended medicament dose based on past trends allows the glucose level control system 1600 to adjust a subject's medicament intake slowly and safely. In some embodiments, the glucose level control system 1600 may continue to increase or decrease the size of the recommended medicament doses to facilitate effectively managing the subject's disease (e.g., the subject's glucose levels stay within a desired and expected range).

In some embodiments, the glucose level control system 1600 may generate a backup therapy protocol for a subject. The backup therapy protocol may be based at least in part on one or more user-specified medicament-related inputs and/or a subject's glucose level data. For example, the backup therapy protocol may be based at least in part on one or more user-specified quantitative amounts of medicament. Alternatively, or in addition, the backup therapy protocol may be based at least in part on one or more stored indications of previously recommended and/or administered medicament doses. In some embodiments, the glucose level control system 1600 may update the backup therapy protocol based at least in part on a user-specified medicament-related input and/or the subject's glucose level data. Alternatively, or in addition, the glucose level control system 1600 may update the backup therapy protocol based at least in part on one or more indications of previously recommended and/or administered medicament doses. Examples of systems that can be used to generate or update backup therapy protocols are disclosed in U.S. Pat. No. 10,960,137, issued on Mar. 30, 2021, the entire contents of which are hereby incorporated by reference herein and made a part of this specification.

In some embodiments, the backup therapy protocol indicates a treatment plan (e.g., indicates one or more recommended medicament doses). Alternatively, or in addition, the backup therapy protocol may indicate recommended settings for an ambulatory medicament device. The glucose level control system 1600 may output the indications of the backup therapy protocol. For example, the glucose level control system 1600 may display the indications of the backup therapy protocol on the display of the glucose level control system 1600 and/or the display 1710 of the smart pen 1602. Alternatively, or in addition, a user may be able to access the indications of the backup therapy protocol using an electronic device separate from the glucose level control system 1600 (e.g., a smartphone, laptop, smartglasses, tablet, server, or other remote computing device), enabling the electronic device to display the indications of the backup therapy protocol. In some embodiments, the backup therapy protocol may be stored in a memory 540 of the glucose level control system 1600, a remote storage, and/or a removable memory device.

Using the indications of the backup therapy protocol, a user can perform injection therapy (e.g., via the smart pen 1602) according to the indicated treatment plan. Alternatively, or in addition, a user can use the indications of the backup therapy protocol to initialize a new, or another, glucose level control system 1600. For example, a user can upload the indications of the backup therapy protocol to an ambulatory medicament device or enter values from the indications of the backup therapy protocol via a user interface to update the default control parameters of the ambulatory medicament device. Thus, a user can quickly configure a new ambulatory medicament device based at least in part on a subject's treatment plan.

In some embodiments, as shown by arrow 1810, the glucose level control system 1600 can perform the process 1800 multiple times. For example, during a subsequent iteration of the process 1800, the glucose level control system 1600 may receive additional glucose level data at the block 1802. Further, the glucose level control system 1600 may receive additional user-specified medicament-related inputs (e.g., a size of a second medicament dose that a user intends to administer to the subject, or macronutrient data for food to be consumed, etc.) at the block 1804. The glucose level control system 1600 can determine one or more recommended doses of medicament based on the additional user-specified medicament-related input and/or the additional glucose level data at the block 1806, which may be output at the block 1808.

In some embodiments, the glucose level control system 1600 can determine whether the user has administered the recommended medicament dose to the subject. For example, a user may interact with a user interface of the glucose level control system 1600 or the smart pen 1602 to indicate that the recommended medicament dose has been administered to the subject. For example, the glucose level control system 1600 can prompt the user via a message on a display to confirm that the recommended medicament dose has been administered. Alternatively, or in addition, the glucose level control system 1600 may determine whether the user has administered the recommended medicament dose to the subject based at least in part on the glucose levels of the subject over a particular time period. The time period may be on the order of minutes to hours. The glucose level control system 1600 may compare the glucose level of the subject to an expected glucose level. The expected glucose level of the subject may be based at least in part on historical glucose data of the subject. For example, the glucose level control system 1600 may determine that the recommended medicament dose has not been administered to the subject if the subject's glucose levels are not reduced by at least a certain amount (e.g., 20 ml/dL to 60 ml/dL) during the time period (e.g., one to six hours) after an indication of the recommended medicament dose has been outputted to the user.

As described above, the glucose level control system 1600 may create one or more therapy periods. A therapy period may be one or more minutes, hours, or days. In some embodiments, the glucose level control system 1600 determines a therapy period based on the user's behavior or the subject's behavior. For example, the glucose level control system 1600 may start a new therapy period after receiving an indication that the user has administered the recommended medicament dose to the subject. Alternatively, or in addition, the glucose level control system 1600 may start a new therapy period after the subject has consumed a meal.

In some embodiments, the glucose level control system 1600 adapts one or more control parameters in response to the subject's past glucose levels (e.g., glucose levels associated with one or more prior therapy periods). For example, the glucose level control system 1600 may monitor the subject's glucose levels during a first therapy period where a recommended medicament dose has been administered. If the subject's glucose levels leave a desired range (e.g., a range determined by a physician or healthcare provider) during the first therapy period, the glucose level control system 1600 may adjust one or more control parameters. The control parameters may be adapted to better maintain the subject's glucose levels within the desired range. The glucose level control system 1600 may use the updated control parameters to determine the recommended medicament dose for a second therapy period.

Alternatively, or in addition, the glucose level control system 1600 may adapt one or more control parameters in response to the subject's current glucose levels (e.g., glucose levels associated with the current therapy period) and past glucose levels (e.g., glucose levels associated with one or more prior therapy periods). For example, the glucose level control system 1600 may monitor the subject's glucose levels during a first therapy period where a recommended medicament dose has been administered and a second therapy period (e.g., the current therapy period). If the subject's glucose levels leave a desired range (e.g., a range determined by a physician or healthcare provider) for more than a threshold period of time during the first or second therapy period, the glucose level control system 1600 may adjust one or more control parameters. The glucose level control system 1600 may use the updated control parameters to determine the recommended medicament dose for the second therapy period.

In some embodiments, the glucose level control system 1600 determines the recommended medicament dose based at least in part on one or more previously recommended but not administered medicament doses (e.g., missed medicament doses). The glucose level control system 1600 may accumulate (e.g., add) the missed medicament doses together into an accumulated medicament dose. It should be noted that the glucose level control system 1600 may repeat the accumulation process multiple times. For example, if a user fails to administer a first recommended medicament dose, the first recommended medicament dose may be added to the accumulated medicament dose when a second recommended medicament dose is determined. If the user fails to administer the second recommended medicament dose, the second missed recommended medicament dose can be added to the accumulated medicament dose and accounted for in the third recommended medicament dose. It should be noted that the glucose level control system 1600 may output an indication of each of the recommended medicament doses.

In some embodiments, the glucose level control system 1600 determines the recommended medicament dose based on an accumulated medicament dose of one or more missed correction insulin doses. For example, if a user fails to administer two recommended correction insulin doses of three units of insulin, the glucose level control system 1600 may determine a third recommended medicament dose of at least nine units of insulin (e.g., an accumulated medicament dose of six units plus a current correction insulin dose of three units). Alternatively, the accumulated medicament dose may be one or more basal insulin doses. In some embodiments, the glucose level control system 1600 determines a recommended medicament dose based on recommended medicament doses that have not been administered due to a broken, malfunctioning, or missing ambulatory medicament device. For example, if the basal rate of the ambulatory medicament device is half a unit of insulin per hour, and the ambulatory medicament device has been inoperable for six hours, the glucose level control system 1600 may determine a recommended medicament dose of three units of insulin. In some embodiments, the accumulated medicament dose may include one or more missed correction medicament doses and one or more missed basal medicament doses. For example, if a user failed to administer a correction medicament dose of four units of insulin and basal medicament doses of half a unit of insulin per hour for the last six hours, the glucose level control system 1600 may determine a recommended medicament dose of at least seven units of insulin.

In some embodiments, the glucose level control system 1600 determines a recommended medicament dose based at least in part on a reduced accumulated medicament dose. The glucose level control system 1600 may have a max medicament dose size for the accumulated medicament dose. In some cases, the max medicament dose size prevents a subject from receiving a potentially dangerous dose of medicament (e.g., a medicament dose that is likely to result in adverse health effects). For example, in some cases, the max medicament dose size may prevent a subject from receiving a potentially unsafe dose of insulin (e.g., an insulin dose that may cause an unsafe drop in the subject's glucose level and/or cause the subject to experience a hypoglycemic episode). In some cases, the max medicament dose size may vary based on the physiological characteristics of the subject, available data (e.g., glucose level) of the subject, and/or the recency of the available data. By basing the max medicament dose size on physiological characteristics of the subject, the available data, and/or the recency of the available data, the risk of hypoglycemia or other unsafe conditions due to too large of a dose of medicament may be reduced.

In some embodiments, the glucose level control system 1600 may accumulate missed medicament doses until the size of the accumulated medicament dose reaches the max accumulated medicament dose size (e.g., 10 units of either fast-acting insulin or long-acting insulin). If the user fails to administer a recommended medicament dose to the subject after the accumulated medicament dose has reached the max accumulated medicament dose size, the glucose level control system 1600 may not accumulate the missed medicament dose to the accumulated medicament dose. For example, if a user fails to administer two recommended medicament doses of four units of insulin and the max accumulated medicament dose size is five units of insulin, the glucose level control system 1600 may determine a third recommended medicament dose of nine units of insulin (e.g., a reduced accumulated medicament dose of five units plus a current medicament insulin dose of four units). In other words, the glucose level control system 1600 may accumulate a reduced accumulated medicament dose and a current medicament dose to determine the current recommended medicament dose. Alternatively, or in addition, the glucose level control system 1600 may alert a user of the missed medicament doses and/or alert the user that the accumulated medicament dose has reached a max accumulated medicament dose size.

In some cases, missed medicament doses become less relevant to a subject's current glucose levels over time. Thus, the glucose level control system 1600 may reduce the accumulated medicament dose over time. For example, the glucose level control system 1600 may reduce the accumulated medicament dose by a percentage (e.g., 5%-40%) every hour. The glucose level control system 1600 may adjust the reduction percentage over time to maintain the subject's glucose levels more effectively within a desired range, such as between 60 mg/dL and 170 mg/dL. In some embodiments, the glucose level control system 1600 outputs an indication of the recommended medicament dose that is based at least in part on a reduced accumulated medicament dose. The glucose level control system 1600 may alert the user that the recommended medicament dose is based at least in part on an accumulated medicament dose. For example, the display of the glucose level control system 1600 may display an electronic message that reads, “the recommended medicament dose is larger compared to previous recommended medicament doses due to one or more missed medicament doses.”

In some cases, the glucose level control system 1600 may automatically detect one or more missed medicament doses based at least in part on the subject's glucose level data. Alternatively, or in addition, the glucose level control system 1600 may automatically detect missed medicament doses based at least in part on the rate of change of the subject's glucose level over time. For example, if the subject's glucose level is not falling or is falling slower than expected after a recommended medicament dose is outputted (e.g., the rate of change of the subject's glucose level over time does not correspond to the recommended medicament dose's size and the subject's insulin sensitivity), the glucose level control system 1600 may determine that the user has failed or neglected to administer one or more recommended medicament doses to the subject.

The glucose level control system 1600 may determine a recommended medicament dose based at least in part on the one or more automatically detected missed medicament doses. For example, if the glucose level control system 1600 detects a missed medicament dose, the glucose level control system 1600 may determine a larger recommended medicament dose than if no missed medicament doses had been detected. It should be noted that in some embodiments the recommended medicament dose may not be an accumulation of the missed medicament doses (e.g., the missed medicament doses may affect one or more control parameters but are not added together, or not added together in their entirety, to determine the recommended medicament dose).

In some embodiments, the glucose level control system 1600 alerts the user that one or more missed medicament doses have been detected. In some cases, the glucose level control system 1600 may prompt the user (e.g., via the user interface) to confirm whether a medicament dose was administered but not detected or reported, or if the medicament dose was missed or not administered. The determination of whether a medicament dose was missed may be uncertain due to a broken or malfunctioning sensor (e.g., glucose level sensor). Alternatively, or in addition, the determination of whether the medicament dose was missed may be uncertain due to an untimely or missing glucose level measurement (e.g., the user failed to enter one or more isolated glucose measurement during a therapy period) or a failure by the user to report or confirm an administered medicament dose. In some cases, the glucose level control system 1600 accounts for the missed medicament dose when generating a recommended medicament dose (e.g., a second medicament dose). Accounting for the missed medicament dose may include modifying the second recommended medicament dose. Alternatively, the second medicament dose may inherently account for the missed medicament dose based on the state of the subject (e.g., glucose level) corresponding at least in part to the missed medicament dose. In cases where it is uncertain that a medicament dose has been missed, the missed medicament dose may be excluded from the therapy data used to determine a recommended medicament dose. Alternatively, the missed medicament dose may be inherently excluded or account for based on the state of the subject (e.g., glucose level) corresponding at least in part to the missed medicament dose. Moreover, if the user fails to confirm the determination that a medicament dose was missed, the glucose level control system 1600 may exclude the missed medicament dose when determining a recommended medicament dose (e.g., the control parameters may not be affected by the missed medicament doses). Thus, in some embodiments, the glucose level control system 1600 may exclude faulty therapy data by requiring a user to confirm that the therapy data is correct.

In some cases, the glucose level control system 1600 can perform operations associated with the blocks 1802-1808 in response to certain events. For example, the process 1800 can be initiated during the glucose level control system's 1600 startup procedure or after the glucose level control system's 1600 settings have been updated. Further, the process 1800 can be initiated at particular time intervals or with a particular schedule. For example, the process 1800 may be repeated every 30 minutes, each hour, every 6 hours, every 12 hours, each day, every other day, any time between the aforementioned examples, or with any other frequency. In some embodiments, the glucose level control system 1600 performs operations associated with the process 1800 during mealtimes or times of regular food-intake (e.g., 8 am, 12 pm, and 5 pm, etc.). Alternatively, or in addition, the process 1800 can be initiated after a user performs a certain action (e.g., the user inputs an isolated glucose measurement into the glucose level control system 1600 or provides a user-specified medicament-related input). In some embodiments, the process 1800 may be performed when the subject's glucose levels hit a certain glucose level threshold. For example, if a subject's glucose level is in a certain threshold, the glucose level control system 1600 may alert the user and request a user-specified medicament-related input. Overall, process 1800 can generate one or more medicament dose recommendations for controlling the glucose level of a subject.

### Recommendation Process for a Non-Automated Medicament Dose

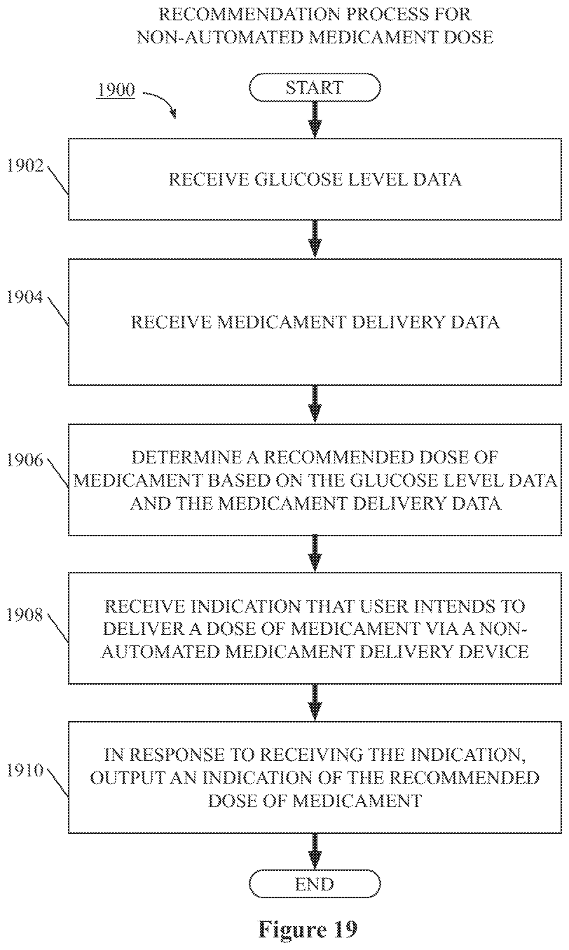

FIG. 19 presents a flowchart of an example recommendation process 1900 for a non-automated medicament dose in accordance with certain embodiments. The process 1900 may be performed by any system that can generate a medicament dose recommendation for controlling the glucose level of a subject. For example, the process 1900 may be performed by the glucose level control system 1600, an ambulatory medicament system, an ambulatory medical device, or an ambulatory medical pump. In some embodiments, the process 1900 may be at least partly performed by one or more elements of the glucose level control system 1600, such as the processor 530, one or more controllers 518, the memory 540, or an input/output circuitry 532. In some cases, at least certain operations of the process 1900 may be performed by a separate computing system that receives therapy data corresponding to the subject. Although one or more different systems may perform one or more operations of the process 1900, to simplify discussions and not to limit the present disclosure, the process 1900 is described with respect to particular systems.

The process 1900 begins at block 1902 where, for example, the glucose level control system 1600 receives glucose level data associated with the glucose level of the subject. As described above, the glucose level control system 1600 may receive the glucose level data from a glucose sensor that is operatively connected to the subject (e.g., CGM sensor) and/or from isolated glucose measurements. In some embodiments, the block 1902 may include one or more of the embodiments previously described with respect to the block 1802.

At block 1904, the glucose level control system 1600 receives medicament delivery data. In some embodiments, the medicament delivery data is associated with glucose level control agents that have been or may be administered to the subject. For example, the medicament delivery data may include a quantity of medicament delivered, a timing of delivery of medicament, a rate of medicament delivery, a type of medicament delivered (e.g., FAI or LAI), or any other type of medicament delivery information that may facilitate determining a state of the subject and/or future medicament delivery. The medicament delivery data may be received via user interaction with a user interface. For example, the glucose level control system 1600 may prompt the user via the user interface to enter the medicament delivery data, for example, via the user interface or by causing transmission of the medicament delivery data to the glucose level control system 1600. In some embodiments, a remote storage device transmits the medicament delivery data to the glucose level control system 1600. Alternatively, or in addition, the medicament delivery data may be received from one or more sensors.

In some embodiments, the medicament delivery data includes a log of past medicament deliveries. Alternatively, or in addition, the log of past medicament deliveries may include the type, size, frequency, concentration of the past medicament deliveries, and/or any other type of medicament delivery data that may be recorded by a tracking or log system. Each delivery in the log may include a time stamp indicating when the medicament dose was administered to the subject. The time stamp may include the time, date, and/or therapy period. In some embodiments, the medicament delivery data includes a log of previously recommended medicament doses. In some embodiments, the medicament delivery data may include whether the previously recommended medicament doses were administered to the subject, if a modified dose was administered, or if a previously recommended medicament dose was omitted from the subject's treatment. Alternatively, or in addition, the log of previously recommended medicament doses may include the type, size, and/or concentration of the previously recommended medicament doses. In some embodiments, the medicament delivery data may include one or more indications of insulin delivery events (e.g., recommended or administered insulin doses). The insulin delivery events may be long-acting insulin events, fast-acting insulin events, or delivery events corresponding to any other type of insulin. Further, the delivery events may be associated with food-intake, exercise, a glycemic excursion, basal, or any other type of delivery event. In some embodiments, the medicament delivery data includes a certain number of indications of past medicament delivery events (e.g., indications of the past 5-20 recommended or administered medicament doses). It should be noted that the medicament delivery data can include indications of medicament delivery events delivered via an ambulatory medicament device, the smart pen 1602, and/or any other delivery method.

In some embodiments, the medicament delivery data includes an indication of the insulin on board of the subject (e.g., the amount of insulin that is still active or predicted to still be active in the subject's body after a medicament bolus). The medicament delivery data may include the total amount of insulin on board, the amount of fast-acting insulin on board, the amount of long-acting insulin on board, or the amount of any other type of insulin on board (e.g., rapid-acting insulin, intermediate-acting insulin, ultra-long-acting insulin, etc.). Alternatively, or in addition, the medicament delivery data may include past amounts of the insulin on board of the subject. For example, the medicament delivery data may include data corresponding with the insulin on board of the subject for a therapy period.

In some embodiments, the medicament delivery data includes one or more values of one or more control parameters used by a pharmacokinetic model. As described above, the control parameters of the pharmacokinetic model may be at least partly based on the type of insulin delivered or recommended, the glucose level of the subject, the physiological characteristics of the subject, the health condition of the subject, one or more physiological parameters of the subject, the time of the administration or recommendation of the medicament dose, the location at which the infusion set is placed, the amount of insulin administered and the like. Alternatively, or in addition, control parameters used by the pharmacokinetic model may be based at least partly on the subject's medicament absorption rate (e.g., how quickly the medicament is absorbed into the subject's blood plasma or the amount of time the insulin remains effective with the subject). In some embodiments, the control parameters of the pharmacokinetic model may be at least partly based on how quickly the subject's body processes the medicament.

In some embodiments, the glucose level control system 1600 receives the medicament delivery data continuously. Alternatively, the glucose level control system 1600 may receive medicament delivery data on an intermittent basis. For example, the glucose level control system 1600 may receive medicament delivery data every minute, every five minutes, every thirty minutes, every hour, every five hours, once a day, etc.). In some embodiments, the medicament delivery data can include data recorded during a particular time period. The time period may be on the order of minutes, hours, days, or therapy periods.

At block 1906, the glucose level control system 1600 determines a recommended medicament dose based on the glucose level data and the medicament delivery data. In some embodiments, the processor 530 of the glucose level control system 1600 uses a control algorithm to determine a recommended medicament dose for the subject. In some embodiments, the control algorithm is a pharmacokinetic model or is at least partly based on a pharmacokinetic model. Additionally, the control algorithm may be configured to control the glucose levels of a subject (e.g., by determining and outputting indications of recommended medicament doses). As described above, the control algorithm can generate at least one of a recommended basal dose, a recommended correction dose, and/or a recommended meal or food-intake dose. The recommended medicament dose can include regulatory agents, such as fast-acting insulin and/or long-acting insulin. Further, the recommended medicament dose may be determined using a model-predictive control (MPC) or any other type of control algorithm, such as one or more of the control algorithms disclosed in the Controller Disclosures. In some embodiments, the control algorithm can generate a recommended medicament dose of a counter-regulatory agent, such as glucagon. Alternatively, or in addition, the recommended medicament dose can be a dose of carbohydrate therapy (e.g., the consumption of carbohydrates to address hypoglycemic events or potential hypoglycemic events). For example, the glucose level control system 1600 may determine a recommendation for the subject to consume a specified amount of food. The recommended amount of food may be a particular food item, particular food type, particular quantity of food, or a particular amount of macronutrients. Alternatively, or in addition, the recommendation may be for a particular amount of counter-regulatory agent (e.g., glucagon).

In some embodiments, the glucose level control system 1600 may use the glucose level data and/or the medicament delivery data as control parameter values or to determine control parameter values used by the control algorithm. For instance, one or more control parameters can be set based at least in part on the subject's glucose level data. For example, one or more control parameters may be set based at least in part on the subject's glucose level history and/or on the subject's current glucose level. Alternatively, or in addition, one or more control parameters can be based at least in part on the medicament delivery data. For example, one or more control parameters may be set based at least in part on a one or more indications of medicament delivery events, an indication of the insulin on board of the subject, an indication of insulin absorption rate by the subject, and/or any other data that may be used to set one or more control parameters. Similarly, one or more control parameters may be set based at least in part on any other medicament delivery data.

In some embodiments, the glucose level control system 1600 may determine a recommended medicament dose based on one or more control parameters. Further, the recommended medicament dose may be determined based at least in part on the subject's glucose level data, glucose level trends, and/or glucose level history. In some embodiments, the block 1906 may include one or more of the embodiments previously described with respect to the block 1806.

Alternatively, or in addition, the glucose level control system 1600 may determine the recommended medicament dose based at least in part on the medicament delivery data. For example, the glucose level control system 1600 may use one or more indications of medicament delivery events to determine the recommended medicament dose. The glucose level control system 1600 may determine a recommended medicament dose based at least in part on the average size of the medicament delivery events. For example, if the average size of the previously administered medicament doses is five units of long-acting insulin, the glucose level control system 1600 may determine a recommended medicament dose that is about five units of long-acting insulin. Alternatively, or in addition, the glucose level control system 1600 may determine a recommended medicament dose based at least in part on the trends of the medicament delivery events. For example, if the size of the previously administered medicament doses is trending upward (e.g., each administered medicament dose is 5% larger than the previously administered medicament dose), the glucose level control system 1600 may determine a recommended medicament dose that continues the trend (e.g., the recommended medicament dose may be about 5% larger than the previously administered medicament dose). Determining a recommended medicament dose based on past trends allows the glucose level control system 1600 to adjust a subject's medicament intake slowly and safely. In some embodiments, the glucose level control system 1600 may continue to increase or decrease the size of the recommended medicament doses until the subject's disease is effectively managed (e.g., the subject's glucose levels stay within a desired and expected range). Moreover, in some cases, the evaluation of past medicament delivery events may include consideration of the glucose level of the subject and the rate of change of the glucose level of the subject. Thus, a recommended dose of medicament may be increased or decreased based at least in part on the subject's glucose level and/or changing glucose level.

In some embodiments, the glucose level control system 1600 may determine the recommended medicament dose based at least in part on an indication of the insulin on board of a subject. For example, the glucose level control system 1600 may determine a recommended medicament dose that maintains the subject's insulin on board below a maximum insulin on board (“JOB”) value or threshold reducing the risk of hypoglycemia. In some embodiments, if the insulin on board of the subject is high (e.g., more than 50% or 75% of the maximum insulin on board threshold), the glucose level control system 1600 may determine a smaller recommended medicament dose compared to a recommended medicament dose when a subject's insulin on board value is low (e.g., below 50% or 75% of the maximum insulin on board threshold). As another example, the recommended dose of medicament may be reduced to prevent the IOB from exceeding the maximum insulin on board threshold. Alternatively, or in addition, the control algorithm executed by the glucose level control system 1600 may account for a maximum IOB threshold in generating a recommended dose of medicament to prevent or reduce the possibility of the IOB exceeding the maximum IOB threshold.

In some embodiments, the glucose level control system 1600 may use one or more values of one or more control parameters used by a pharmacokinetic model to determine the recommended medicament dose. For example, if the subject is physically active the glucose level control system 1600 may determine a smaller recommended medicament dose than if the subject has a sedentary lifestyle. In some cases, imminent exercise (e.g., exercise within the next four hours) may affect one or more control parameters of a pharmacokinetic model. For example, a workout may temporarily (e.g., for the next 6-48 hours) increase a subject's insulin sensitivity. Thus, in some embodiments, the glucose level control system 1600 may determine a smaller recommended medicament dose if the user indicates that the subject may exercise soon (e.g., within the next two hours) or has recently exercised (e.g., within the past twelve hours). In some embodiments, the glucose level control system 1600 may determine recommended medicament doses at different times based on the control parameters used by the pharmacokinetic model. For example, if the glucose level control system 1600 determines, using a pharmacokinetic model, that medicament doses administered at a certain time (e.g., morning) maintain the subject's glucose levels within a desired range more effectively than medicament doses administered at other times, the glucose level control system 1600 may determine a recommended medicament dose during that certain time (e.g., morning). Alternatively, or in addition, as described above, the glucose level control system 1600 may determine a recommended medicament dose based on the physiological data of a subject.

At block 1908, the glucose level control system 1600 receives an indication that the user intends to deliver a dose of medicament via a non-automated medicament delivery device. In other words, the medicament delivery device may require user interaction to deliver medicament. For example, the medicament delivery device may be a smart pen 1602. The glucose level control system 1600 may receive the indication via a dosing control interface. The dosing control interface may be a graphical user interface (GUI) that may be displayed on a display. One non-limiting example user interface is illustrated in FIG. 22 and described in more detail below. In some embodiments, the user specifies, via the dosing control interface, the medicament delivery device (e.g., insulin pen) used to administer the recommended medicament dose. In some embodiments, the glucose level control system 1600 prompts the user to confirm delivery of the medicament and/or the device used to administer the medicament. For example, the glucose level control system 1600 may use an alert (e.g., a flashing light or a sound) to remind the user to administer insulin.

At block 1910, the glucose level control system 1600 may output an indication of the recommended medicament dose in response to receiving the indication that the user intends to deliver the recommended medicament dose via a non-automated delivery device. For example, the glucose level control system 1600 can output the indication of the recommended medicament dose to a display of the glucose level control system 1600. Alternatively, or in addition, the glucose level control system 1600 can transmit the indication of the recommended medicament dose to the smart pen 1602 enabling the smart pen 1602 to display the recommended medicament dose. The smart pen 1602 can display the indication of the recommended dose of medicament on the display 1710 of the smart pen 1602. It should be noted that the glucose level control system 1600 can output the indication of the recommended medicament dose according to any previously described process or embodiment (e.g., block 1808).

A user can use the outputted indication to configure the smart pen 1602 according to the recommended medicament dose (e.g., configure the smart pen 1602 to administer the recommended medicament dose). Alternatively, the smart pen 1602 may be automatically configured based on the recommended medicament dose. For example, the smart pen 1602 may automatically configure itself to deliver the recommended medicament dose in response to receipt of the indication of the recommended medicament dose from, for example, the glucose level control system 1600. Accordingly, if the user desires to administer the recommended medicament dose, the user can initiate administration of the automatically configured medicament dose without manually configuring the smart pen 1602 with the recommended medicament dose. For example, the glucose level control system 1600 can generate and send a dose control signal based on the recommended medicament dose to the smart pen 1602. In some embodiments, the dose control signal automatically configures the smart pen 1602 according to the determined recommended medicament dose. In some cases, a user may confirm the recommended medicament dose using a dosing control interface (e.g., a user interface) of the glucose level control system 1600 and/or smart pen 1602. The glucose level control system 1600 may configure the smart pen 1602 to administer the recommended medicament dose in response to the confirmation by the user of the recommended medicament dose. Thus, the glucose level control system 1600 can reduce user errors (e.g., by reducing user inputs) and streamline the medicament delivery process when using an insulin pen or smart pen 1602.

In some embodiments, the smart pen 1602 cannot administer a dose of medicament without receiving a confirmation of the recommended medicament dose via a user interface of the smart pen 1602 or the glucose level control system 1600. For example, the smart pen 1602 may have a safety lock that prevents the user from administering the recommended medicament dose unless the smart pen 1602 has received a confirmation from the dosing control interface of the glucose level control system 1600. In some embodiments where the smart pen 1602 includes the safety lock, the smart pen 1602 may request a user confirm the type, size, and/or concentration of the recommended medicament dose prior to administering the medicament dose. The confirmation may provide a safety check to prevent the glucose level control system 1600 from configuring the smart pen 1602 with an incorrect dose of medicament (e.g., a dose of medicament that is not consistent with the subject's treatment plan). Alternatively, or in addition, the safety lock may prevent users who have diminished faculties or are children from confirming the recommended medicament dose. In some embodiments, only an authorized user may confirm the recommended medicament dose. For example, the glucose level control system 1600 may identify an authorized user (e.g., the subject or a caregiver of the subject) by requiring the user to insert a password or by any other verification method.

In some embodiments, block 1908 and block 1910 are optional. For example, the glucose level control system 1600 may automatically (e.g., without receiving user-input or confirmation) output the indication of the recommended medicament dose to a display (e.g., the display of the glucose level control system 1600 or the smart pen 1602). Alternatively, or in addition, the glucose level control system 1600 may automatically transmit a dose control signal to an automated ambulatory medicament device. In some embodiments, the glucose level control system 1600 automatically configures the smart pen 1602 or another non-automated delivery device via a dose control signal without receiving user-input indicating the delivery device and/or confirmation. As another example, the operations associated with the blocks 1902-1906 may be repeated one or more times. As the operations associated with the block 1906 are repeated, the recommended dose of medicament may be updated or modified. Upon receiving an interaction with a user interface indicating that a user intends to administer the medicament, the operations associated with the blocks 1908 and 1910 may be performed. Thus, in some cases, operations associated with the blocks 1902-1906 may be performed a plurality of times before the operations associated with the blocks 1908 and 1910 are performed.

In some cases, the glucose level control system 1600 can perform operations associated with the blocks 1902-1910 in response to certain events. For example, glucose level control system 1600 can initiate the process 1900 in response to receiving medicament delivery data. Alternatively, or in addition, the process 1900 can be initiated during the glucose level control system's 1600 startup procedure or after the glucose level control system's 1600 settings have been updated. Further, the process 1900 can be initiated at particular time intervals or with a particular schedule. For example, the process 1900 may be repeated every 30 minutes, each hour, every 6 hours, every 12 hours, each day, every other day, any time between the aforementioned examples, or with any other frequency. In some embodiments, the glucose level control system 1600 performs operations associated with the process 1900 during mealtimes or times of regular food-intake (e.g., 8 am, 12 pm, and 5 pm, etc.). Alternatively, or in addition, the process 1900 can be initiated after a user performs a certain action (e.g., the user inputs an isolated glucose measurement into the glucose level control system 1600). In some embodiments, the process 1900 may be performed when the subject's glucose levels hit a certain glucose level threshold. For example, if a subject's glucose level is in a certain threshold, the glucose level control system 1600 may alert the user and request the user to specify a delivery device type to be used for the recommended medicament dose. Overall, process 1900 can generate one or more medicament dose recommendations for controlling the glucose level of a subject.

### Medicament Dose Reminder Process

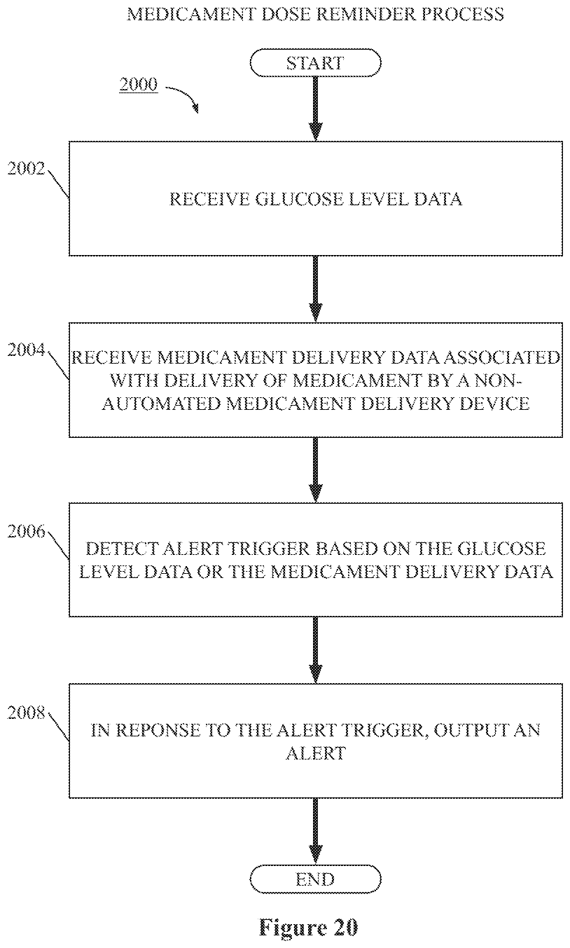

FIG. 20 presents a flowchart of an example medicament dose reminder process 2000 in accordance with certain embodiments. The process 2000 may be performed by any system that can generate a dose reminder for administering a dose of medicament using a medicament delivery device that may require user interaction with a dosing control interface (e.g., a user interface) to deliver medicament. In other words, in some cases, the medicament delivery device may be a device that requires manual or user interaction to administer the medicament. In other cases, the medicament delivery device may include options for automatic, manual, or automatic and manual delivery of medicament. In some non-limiting examples, the process 2000 may be performed by the glucose level control system 1600, an ambulatory medicament system, an ambulatory medical device, or an ambulatory medical pump. In some embodiments, the process 2000 may be at least partly performed by one or more elements of the glucose level control system 1600, such as the processor 530, one or more controllers 518, the memory 540, or an input/output circuitry 532. In some cases, at least certain operations of the process 2000 may be performed by a separate computing system that receives therapy data corresponding to the subject. Although one or more different systems may perform one or more operations of the process 2000, to simplify discussions and not to limit the present disclosure, the process 200 is described with respect to particular systems.

The process 2000 begins at block 2002 where, for example, the glucose level control system 1600 receives glucose level data associated with the glucose level of the subject. As described above, the glucose level control system 1600 may receive the glucose level data from a glucose sensor that is operatively connected to the subject (e.g., CGM sensor) and/or from one or more isolated glucose measurements. In some embodiments, the block 2002 may include one or more of the embodiments previously described with respect to the block 1802 or the block 1902.

At block 2004, the glucose level control system 1600 receives medicament delivery data associated with delivery of medicament by a non-automated or manual medicament delivery device. As described above, the medicament delivery data may be received via user interaction with a user interface. Alternatively, or in addition, the glucose level control system 1600 may retrieve medicament delivery data from a memory 540 and/or a remote storage device. The medicament delivery data may include an indication of medicament type (e.g., insulin, an insulin analog, a fast-acting insulin, a long-acting insulin, a counter-regulatory agent, etc.), timing of one or more medicament delivery events, quantity of medicament delivered during each medicament delivery event, or any other type of medicament delivery information that may facilitate determining a state of the subject and/or future medicament delivery. In some embodiments, the medicament delivery data is associated with glucose level control agents that have been or may be recommended and/or administered to the subject. Alternatively, or in addition, the non-automated medicament delivery device may require user interaction to deliver medicament. For example, the medicament delivery device may be a smart pen 1602. In some cases, the glucose level control system glucose level control system 1600 may automatically deliver the medicament in response to a user confirmation of the medicament dose. In some embodiments, the block 2004 may include one or more of the embodiments previously described with respect to the block 1904.

At block 2006, the glucose level control system 1600 detects one or more alert triggers based at least in part on the glucose level data and/or the medicament delivery data. The glucose level control system 1600 detects the occurrence of an alert trigger by at least determining whether one or more trigger criteria are satisfied. In some embodiments, a trigger criterion may be satisfied if the glucose level control system 1600 determines that a dosing frequency is less than a dosing frequency threshold. For example, if the dosing frequency threshold is three medicament doses per day, and a subject has received less than three medicament doses near the end of the day (e.g., one to six hours before the end of the day), the trigger criterion may be satisfied. Similarly, if the dosing frequency is one medicament dose every four hours, the trigger criterion may be satisfied once a four-hour period has passed without the subject receiving a medicament dose. The frequency threshold may be any number of medicament doses during a therapy period, and may vary based on the type of medicament or the duration of action of the medicament (e.g., 6 hours, 12 hours, 24 hours, 48 hours, 72 hours, etc.). For example, the frequency threshold may be zero, five, ten, twenty, or more than twenty medicament doses of fast-acting insulin during a therapy period. In some embodiments, the therapy period may be a time period of minutes, hours, or days. Alternatively, or in addition, the therapy period may be a period between two events. For instance, a therapy period may be a period between meals or the period between when a subject wakes up and goes to sleep. In some embodiments, the therapy period may include one or more of the embodiments previously described with respect to the block 702 or the block 710.

In some embodiments, a trigger criterion may be satisfied if the glucose level control system 1600 determines that a dosing frequency is more than a dosing frequency threshold. For example, a trigger criterion may be satisfied if the dosing frequency threshold is two medicament doses per day and the glucose level control system 1600 receives an indication that a user intends to or has administered the third medicament dose of the day. It should be noted that the glucose level control system 1600 may have different frequency thresholds for different medicament types, sizes, and/or concentrations. Alternatively, or in addition, the glucose level control system 1600 may have multiple frequency thresholds corresponding to one or more trigger criteria.

In some embodiments, a trigger criterion may be satisfied if the glucose level control system 1600 determines that the glucose level of the subject does not satisfy a target setpoint by at least a threshold level. The target setpoint may be a setpoint range with a maximum and/or minimum glucose level. For example, the setpoint range may have a minimum glucose level that corresponds to a glucose level that may cause hypoglycemia or is associated with an above-threshold risk of hypoglycemia in a subject (e.g., a glucose level below 70 mg/dL). The trigger criterion may be satisfied if the subject's glucose level drops below the minimum glucose level. Alternatively, or in addition, the trigger criterion may be satisfied if the subject's glucose level drops below the minimum glucose level of the setpoint range for a period of time (e.g., 30 minutes or more). Alternatively, or in addition, the setpoint range may have a maximum glucose level that corresponds to a glucose level that may cause hyperglycemia in a subject (e.g., a glucose level above 180 mg/dL) or is associated with an increased risk of hyperglycemia. The trigger criterion may be satisfied if the subject's glucose level rises above the maximum glucose level. Alternatively, or in addition, the trigger criterion may be satisfied if the subject's glucose level rises above the maximum glucose level of the setpoint range for a period of time (e.g., 30 minutes or more). The period of time required to satisfy the trigger criterion may change depending on the difference between a target setpoint and the subject's glucose level. For example, the trigger criterion may be satisfied more quickly as the difference between the subject's glucose level and the maximum glucose level increases. For instance, the period of time required to satisfy the trigger criterion may be 30 minutes when the subject's glucose level is more than 20 mg/dL above the maximum glucose level and 1 hour when the subject's glucose level is less than 20 mg/dL above the maximum glucose level.

In some embodiments, the target setpoint is a glucose level that corresponds to a desired glucose level (e.g., a glucose level that corresponds to a subject's treatment plan) for the subject's current condition (e.g., two hours after eating a meal and/or receiving a medicament dose). For example, the target setpoint may be a glucose level of 100 mg/dL for a subject who has not had food-intake in the past six to ten hours (e.g., a fasting subject). When the target setpoint is a specific glucose level, the trigger criterion may be satisfied if the difference between the target setpoint and the subject's glucose level is larger than a threshold level. For instance, the threshold level may be a difference between the target setpoint and the subject's glucose level of 10 mg/dL, 20 mg/dL, 30 mg/dL or larger than 30 mg/dL. In some embodiments, the trigger criterion may be satisfied if the difference between the target setpoint and the subject's glucose level is larger than a threshold level for a period of time (e.g., 1 hour or more). It should be noted that the glucose level control system 1600 may have multiple target setpoints and/or setpoint ranges corresponding to one or more trigger criteria.

In some embodiments, a trigger criterion may be satisfied if the glucose level control system 1600 determines that a time since a prior medicament dose exceeds a dosing time threshold. For example, if the dosing time threshold for fast-acting insulin is six hours, the trigger criterion may be satisfied if the subject has not received a fast-acting insulin dose in the last six hours. The dosing time threshold may be a time period of one or more minutes, hours, or days. The glucose level control system 1600 may have one or more dosing time thresholds. For example, the glucose level control system 1600 may have a different dosing time threshold for each medicament type (e.g., fast-acting insulin, long-acting insulin, etc.). Similarly, the glucose level control system 1600 may have different dosing time thresholds for different medicament dose sizes and/or concentrations. For example, a medicament dose of 4 units of fast-acting insulin may have a longer dosing time threshold than a medicament dose of 2 units of fast-acting insulin.

In some embodiments, a trigger criterion may be satisfied if the glucose level control system 1600 determines that the time between a prior medicament dose and an intended or administered dose is less than a dosing time threshold. For example, the trigger criterion may be satisfied if the dosing time threshold is six hours and the glucose level control system 1600 receives an indication that the user intends to administer a medicament dose to a subject four hours after the last medicament dose was administered. As described above, the glucose level control system 1600 may have different dosing time thresholds for different medicament types, sizes, and/or concentrations.

In some embodiments, a trigger criterion may be satisfied if the glucose level control system 1600 determines that a recommended medicament dose satisfies a minimum dose threshold. For example, a trigger criterion may be satisfied if the minimum dose threshold is nine units of long-acting insulin and the glucose level control system 1600 determines a recommended medicament dose of ten units of long-acting insulin. The glucose level control system 1600 may have one or more minimum dose thresholds. For example, the glucose level control system 1600 may have a different minimum dose threshold for each medicament type (e.g., fast-acting insulin, long-acting insulin, etc.). Similarly, the glucose level control system 1600 may have different minimum dose thresholds for different medicament dose sizes and/or concentrations.

In some embodiments, a trigger criterion may be satisfied if the glucose level control system 1600 determines that a change in the glucose level of the subject satisfies a threshold rate of change. For example, a trigger criterion may be satisfied if the threshold rate of change is a glucose level increase of 60 mg/dL per hour and the subject's glucose level increases more than 60 mg/dL per hour. Similarly, a trigger criterion may be satisfied if the threshold rate of change is a glucose level decrease of 60 mg/dL per hour and the subject's glucose level decreases more than 60 mg/dL per hour. It should be noted that the threshold rate of change may vary to accommodate different preferences and therapy treatment plans of a subject. For example, the threshold rate of change may be a glucose level increase and/or decrease of 30 mg/dL, 60 mg/dL, 90 mg/dL, 120 mg/dL, or greater than 120 mg/dL during a certain time period. The time period over which the rate of change is evaluated may vary to accommodate different preferences and therapy treatment plans. For example, the time period may be 30 minutes, 1 hour, 2 hours, 4 hours, 8 hours, or more than 8 hours. In some embodiments, the glucose level control system 1600 may have a different rate of change threshold for a glucose level increase than a glucose level decrease. For example, the threshold rate of change may be an increase of 100 mg/dL per hour and a decrease of 50 mg/dL per hour. Alternatively, or in addition, the glucose level control system 1600 may adjust the threshold rate of change based on the subject's glucose level data and/or the medicament delivery data. For example, the threshold rate of change may be smaller (e.g., a decrease of 30 mg/dL per hour instead of a decrease of 60 mg/dL per hour) if the subject's glucose level is near hypoglycemic levels (e.g., a glucose level of 70 mg/dL or below).

In some embodiments, a trigger criterion may be satisfied if one or more of the previously described trigger criteria are satisfied. For example, a trigger criterion may be satisfied if the glucose level control system 1600 determines that the time since the prior medicament dose exceeds a dosing time threshold and that the recommended dose of the medicament satisfies a minimum dose threshold. In some embodiments, the glucose level control system 1600 may use a control algorithm to determine the thresholds of the trigger criteria (e.g., the dosing frequency threshold, the minimum dose threshold, etc.). For example, the glucose level control system 1600 may determine a dosing frequency threshold based at least in part on the past medicament dosing history of the subject. Similarly, the glucose level control system 1600 may use a control algorithm to determine the glucose level target setpoint or setpoint range of the trigger criteria.

At block 2008, the glucose level control system 1600 outputs an alert in response to one or more alert triggers. The glucose level control system 1600 may transmit the alert to an ambulatory medicament device, such as the smart pen 1602. Alternatively, or in addition, the glucose level control system 1600 may transmit the alert to any authorized electronic device that may be in communication with the glucose level control system 1600. For example, the alert may be transmitted to a subject's or user's smartphone, smartglasses, smartwatch, laptop, tablet, or any other electronic device capable of receiving an alert. In some embodiments, the glucose level control system 1600 may transmit the alert over a network (e.g., the Internet, a cellular network, or any other wide area network). In some embodiments, the alert may be output on the display of the glucose level control system 1600, the display 1710 of the smart pen 1602, and/or the display of another electronic device.

The alert may help a user follow a subject's treatment plan more effectively by reminding the user to administer one or more recommended medicament doses to a subject or informing the user that the subject did not follow the treatment plan. For example, the alert may be a text alert that reads, “dosing time threshold has been exceeded, please administer the recommended dose.” In some embodiments, the text alert includes an indication of the recommended medicament dose. For example, the text alert may read, “please administer the recommended medicament dose of four units of insulin.” In some embodiments, the text alert includes an indication of the accumulation of one or more recommended medicament doses. For example, the text alert may read, “accumulated medicament dose has reached the minimum dose threshold of 10 units of fast-acting insulin, please administer accumulated medicament dose.” As described above, the accumulated medicament dose may be an accumulation of one or more recommended correction medicament doses and/or one or more basal medicament doses.

In some embodiments, the text alert includes a recommended amount of food-intake for the subject. For example, the text alert may read, “glucose level is below the minimum glucose level, please consume a snack or meal.” Alternatively, or in addition, the text alert may specify one or more quantitative characteristics of the food that should be consumed. For example, the text alert may instruct the subject to consume a specific amount of one or more macronutrients. For instance, the text alert may read “please consume a snack with at least 10 g of carbohydrates.” In some embodiments, the glucose level control system 1600 provides treatment plan recommendations to the user via one or more alerts. For example, if a smart-pen-based medicament therapy (e.g., long-acting insulin therapy) is not effectively managing a subject's disease, the glucose level control system 1600 may, via a text alert, recommend that the subject use an ambulatory medicament pump or a patch pump instead of or in combination with the smart pen 1602.

In some embodiments, the alert may be an audio alert. For example, the audio alert may cause the glucose level control system 1600, the smart pen 1602, an ambulatory medicament device, and/or another electronic device to play a sound. Alternatively, or in addition, the alert may be a visual alert. More specifically, the visual alert may cause a light to turn on or flash on the glucose level control system 1600, the smart pen 1602, an ambulatory medicament device, and/or another electronic device. Alternatively, or in addition, the visual alert may cause an icon (e.g., an exclamation mark icon) and/or text to be displayed on the display of the glucose level control system 1600 and/or the display of the smart pen 1602. In some embodiments, the alert may be a haptic alert. For example, the haptic alert may cause the glucose level control system 1600, the smart pen 1602, an ambulatory medicament device, and/or another electronic device to vibrate.

In some embodiments, the glucose level control system 1600 may transmit a dose control signal to an ambulatory medicament device if the glucose level control system 1600 detects the occurrence of one or more alert triggers. For example, the glucose level control system 1600 may transmit a dose control signal that configures the ambulatory medicament device to administer fast-acting insulin, or other type of insulin or insulin analog, if the subject's glucose level is above the maximum glucose level of the setpoint range and/or has an above threshold positive slope. Similarly, the glucose level control system 1600 may transmit a dose control signal that configures the ambulatory medicament device to administer a counter-regulatory agent if the subject's glucose level is below the minimum glucose level of the setpoint range and/or has an above threshold negative slope. In some embodiments, the dose control signal may preconfigure the smart pen 1602 based on a recommended medicament dose when the glucose level control system 1600 detects the occurrence of one or more alert triggers.

Alternatively, or in addition, a glucose level control system 1600 may output the alert using multiple forms of alert or multiple alert types and/or dose control signals when it detects the occurrence of one or more alert triggers. For example, the glucose level control system 1600 may output an alert that has a text, visual, audio, and/or haptic component. In some embodiments, the glucose level control system 1600 may output the alert according to one or more embodiments previously described with respect to the block 1010. Overall, the alert triggers may help the user better manage the subject's disease by helping the user follow the subject's treatment plan more effectively by reminding the user to administer the recommended medicament doses and by recommending adjustments to the subject's treatment plan.

### Meal-Based Medicament Dose Recommendation Process

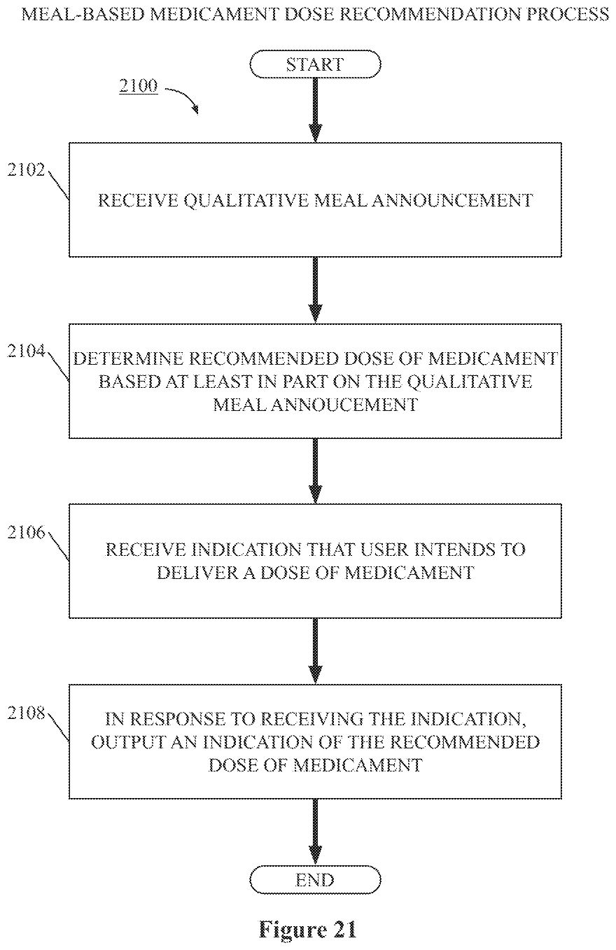

FIG. 21 presents a flowchart of an example meal-based medicament dose recommendation process 2100 in accordance with certain embodiments. The process 2100 may be performed by any system that can generate glucose control therapy recommendations for controlling the glucose level of a subject. For example, the process 2100 may be performed by the glucose level control system 1600, an ambulatory medicament system, an ambulatory medical device, or an ambulatory medical pump. In some embodiments, the process 2100 may be at least partly performed by one or more elements of the glucose level control system 1600, such as the processor 530, one or more controllers 518, the memory 540, or an input/output circuitry 532. In some cases, at least certain operations of the process 2100 may be performed by a separate computing system that receives therapy data corresponding to the subject. The therapy data may include the glucose level data and medicament deliver data corresponding to the subject. Although one or more different systems may perform one or more operations of the process 2100, to simplify discussions and not to limit the present disclosure, the process 2100 is described with respect to particular systems.

The process 2100 begins at block 2102 where, for example, the glucose level control system 1600 receives a qualitative meal announcement. A qualitative meal announcement can include the meal size of a meal or food the subject has consumed or intends to consume. A qualitative meal announcement may include any classification of food that corresponds to or approximates the size or type of food consumed. For example, a user can input that the subject has consumed or may consume a small, medium, or large meal. As another example, the user can indicate that the subject is consuming breakfast, lunch, snack, dinner, supper, etc. In yet another example, the user can indicate that the subject is consuming a high carbohydrate meal, a medium fat meal, a low protein meal, or any other subjective classification of a macronutrient. In some embodiments, the qualitative meal announcement may be supplemented by or replaced by a quantitative meal announcement that may include a measurement of food consumed and/or a measurement of macronutrient in the food consumed (or to be consumed).

In some embodiments, the qualitative meal announcement can include a classification of the meal or food-intake the subject has consumed or intends to consume. For example, the glucose level control system 1600 may classify the food-intake by the carbohydrate level of the food-intake. For example, a user can input that the subject has consumed or may consume a high-carbohydrate meal, a medium-carbohydrate meal, and/or a low-carbohydrate meal. In some embodiments, the glucose level control system 1600 provides descriptions of a high-carbohydrate meal, a medium-carbohydrate meal, and/or a low carbohydrate meal (e.g., through a user manual or via electronic messages) to help a user select the correct meal description. For example, the glucose level control system 1600 may describe a high-carbohydrate meal as a meal with more than 80 grams of carbohydrates, a medium-carbohydrate meal as a meal with 40-80 grams of carbohydrates, and a low-carbohydrate meal as a meal with less than 40 grams of carbohydrates. It should be understood that a user making a qualitative meal announcement may not accurately select the correct size food-intake. For example, the user may believe that a food-intake qualifies as a medium-carbohydrate meal but may in fact be a large-carbohydrate meal. In some embodiments, the meal sizes are defined by a subject's past eating habits. For example, a user may select a high-carbohydrate meal announcement for meals that have more grams of carbohydrates than the subject's average meal, a medium-carbohydrate meal announcement for meals that have about the same amount of carbohydrates as the subject's average meal, and a low-carbohydrates meal announcement for meals that are less grams of carbohydrates than the subject's average meal.

In some embodiments, the qualitative meal announcement may include the type of meal the subject has consumed or intends to consume. For example, a user can input that the subject has consumed or intends to consume breakfast, lunch, dinner, or a snack. The glucose level control system 1600 may provide descriptions to help the user select the correct meal type. For example, the glucose level control system 1600 may define breakfast as the first meal the subject consumes in a day, lunch as the second meal the subject consumes in the day, and a dinner as the third meal the subject consumes in the day. Alternatively, or in addition, breakfast, lunch, dinner, and/or a snack may be defined by meal times and/or meal sizes.

In some embodiments, the glucose level control system 1600 may define the type of meal based at least partly on the demographics (e.g., nationality, religion, ethnicity, etc.) and/or culture of the subject. For example, in Greek culture a dinner may be eaten later (e.g., after 9 pm) compared to dinner in other cultures. Thus, the glucose level control system 1600 may adjust the dinner meal type to correspond more closely to the subject's eating habits. For example, the glucose level control system 1600 may define dinner as a meal eaten between 6 pm to 12 am (as opposed to an American dinner which may be defined as a meal eaten between 4 μm to 8 pm). Alternatively, or in addition, the glucose level control system 1600 may adjust the type of meals available or may create new types of meals to accommodate the subject's preferences, demographics, and/or culture. For example, some religious groups may modify their meal schedules for religious fasts (e.g., Ramadan, Yom Kippur, Lent, etc.). During the fasting period, the glucose level control system 1600 may use different meal types. For example, during Ramadan, the available type of meals may be suhur or predawn meal and iftar or nightly feast instead of breakfast, lunch, and dinner.

In some embodiments, the glucose level control system 1600 may receive the qualitative meal announcement via a dosing control interface. As described above, the dosing control interface may be a graphical user interface (GUI) that may be displayed on a display. One non-limiting example user interface is illustrated in FIG. 22 and described in more detail below. In some embodiments, the dosing control interface may have a one or more input shortcuts for qualitative meal announcements. For example, the dosing control interface may have a dedicated button for a small, medium, or large meal. Thus, the user can input the qualitative meal announcement with a single button press. The button may be either a physical button or a virtual button (e.g., a touchscreen button). Alternatively, or in addition, a user may be able to click a button a number of times to input the qualitative meal announcement. For example, a user may press a button on the dosing control interface one time for a small meal, two times for a medium meal, and three times for a large meal. The input shortcuts may be used to input other meal characteristics as well. For example, a user may input a qualitative meal announcement by clicking a button one time for a low-carbohydrate meal, two times for a medium-carbohydrate meal, and three times for a high-carbohydrate meal. Similarly, a user may input a qualitative meal announcement by clicking a button one time for breakfast, two times for lunch, and three times for dinner. It should be noted that the input shortcut may be any number of clicks for any meal size, characteristic, or type. In some embodiments, the block 2102 may include one or more of the embodiments previously described with respect to the block 1804.

In some embodiments, at block 2102 the glucose level control system 1600 receives the glucose level data associated with the glucose level of the subject. As described above, the glucose level control system 1600 may receive the glucose level data from a glucose sensor that is operatively connected to the subject (e.g., CGM sensor) and/or from one or more isolated glucose measurements. In some embodiments, the block 2102 may include one or more of the embodiments previously described with respect to the block 1802, block 1902, and/or block 2002.

At block 2104, the glucose level control system 1600 may determine a recommended medicament dose based at least in part on the qualitative meal announcement. As described above, the glucose level control system 1600 determines the recommended medicament dose using a control algorithm configured to control the glucose level in the subject. In some embodiments, the glucose level control system 1600 may use the qualitative meal announcement as control parameter values or to determine control parameter values used by the control algorithm. For instance, one or more control parameters can be set based at least in part on the qualitative meal announcement.

In some embodiments, the glucose level control system 1600 may determine a recommended medicament dose at least partly based on the carbohydrate level of the meal a subject has consumed or intends to consume. For example, if the subject's meal (or food-intake) is a high-carbohydrate meal, the glucose level control system 1600 may determine a larger recommended medicament dose compared to if the subject's meal was a medium-carbohydrate meal. Similarly, if the subject's meal is a medium-carbohydrate meal, the glucose level control system 1600 may determine a larger recommended medicament dose compared to if the subject's meal was a low-carbohydrate meal. Alternatively, or in addition, the glucose level control system 1600 may adjust the size of the recommended medicament dose based on the carbohydrate level of the meal a subject has consumed or intends to consume. For example, the glucose level control system 1600 may increase the size of the recommended medicament dose if the user specifies a high-carbohydrate meal and decrease the size of the recommended medicament dose if the user specifies a low-carbohydrate meal. In some embodiments, the glucose level control system 1600 may adapt the size of the recommended medicament dose provided for each carbohydrate level of the meals over time. For example, if the user specifies a carbohydrate level of a meal, and the recommended medicament dose provided does not reduce the subject's glucose level to a desired range (e.g., the subject experiences symptoms of hyperglycemia), the size of the recommended medicament dose may be raised for future meal announcements of the same carbohydrate level. Similarly, if the user specifies a carbohydrate level of a meal, and the recommended medicament dose reduces the subject's glucose levels below a desired range (e.g., the subject experiences hypoglycemic symptoms), the size of the recommended medicament dose may be lowered for future meal announcements of the same carbohydrate level.

In some embodiments, the glucose level control system 1600 may determine a recommended medicament dose that is at least partly based on the meal type (e.g., breakfast, lunch, or dinner) of the meal a subject has consumed or intends to consume. For example, if a subject has consumed or intends to consume a breakfast meal, the glucose level control system 1600 may determine a smaller recommended medicament dose compared to if the subject has consumed or intends to consume a lunch meal. Similarly, if a subject has consumed or intends to consume a lunch meal, the glucose level control system 1600 may determine a smaller recommended medicament dose compared to if the subject has consumed or intends to consume a dinner meal. It should be noted that the recommended medicament dose based at least partly on the different meal types may be adjusted manually by a user or automatically via the control algorithm to represent the subject's eating habits more accurately. For example, some subjects may consume smaller dinner meals than lunch meals. In these circumstances, the glucose level control system 1600 may determine smaller recommended medicament doses for dinner meals than for lunch meals. Similarly, the recommended medicament dose based at least partly on the different meal types may be adjusted manually by a user or automatically via the control algorithm to represent the subject's culture or demographics more accurately. For example, in Spanish culture, the lunch meal may be the main meal of the day. Thus, in such circumstances, the glucose level control system 1600 may determine a larger recommended medicament dose for a lunch meal compared to the other meal types.

As described above, in some embodiments, the glucose level control system 1600 determines a recommended medicament dose based at least partly on the glucose level data of the subject and/or the qualitative meal announcement. Alternatively, or in addition, the glucose level control system 1600 may use the subject's total daily dose of medicament (e.g., insulin) and/or the qualitative meal announcement to determine a recommended medicament dose. For example, the glucose level control system 1600 may use the total daily dose of medicament instead of the subject's glucose level (e.g., when the glucose level is not available) to determine a recommended medicament dose. The subject's total daily dose of medicament is the expected amount of medicament a subject receives in a day according to the subject's treatment plan. Alternatively, the subject's total daily dose of medicament may be an average amount (e.g., over the past two weeks) of medicament a subject receives in a day. The subject's total daily dose of insulin may be especially helpful in determining a recommended medicament dose if the subject's glucose level data is not available. In some embodiments, the glucose level control system 1600 may use the subject's total daily dose of insulin and/or the subject's glucose level as control parameter values or to determine control parameter values used by the control algorithm. For instance, one or more control parameters can be set based at least in part on the subject's total daily dose of insulin and/or the subject's glucose level.

In some embodiments, the glucose level control system 1600 determines the total daily dose of medicament from therapy data (e.g., administered medicament doses) associated with a set of qualified periods. A qualified period may be a time period or a therapy period that has corresponding therapy data that satisfies one or more qualification criteria. The qualification criteria may improve the accuracy of the total daily dose of medicament by removing less reliable therapy data. In some embodiments, the glucose level control system 1600 qualifies one or more time periods from a set of time periods to obtain the set of qualified time periods. The glucose level control system 1600 may retrieve one or more time periods from the memory 540 of the glucose level control system 1600. Alternatively, or in addition, the glucose level control system 1600 may receive one or more time periods from the smart pen 1602 or from any electronic device that is in communication with the glucose level control system 1600.

In some embodiments, a qualification criterion may be satisfied if the glucose level control system 1600 determines that the length of a time period is longer than a minimum length of time. For example, the qualification criterion may be satisfied if the length of the time period is twelve hours and the qualification criterion's minimum length of time is eight hours. Alternatively, or in addition, a qualification criterion may be satisfied if the glucose level control system 1600 determines that the length of a time period is about a certain length of time. For example, the glucose level control system 1600 may qualify a time period to be a qualified day (e.g., a qualified period of about 24 hours) if the time period has a length of about 24 hours (e.g., ±one hour). In some embodiments, a set of qualified periods may be a set of qualified days. For example, the subject's total daily dose of medicament may be the average amount of medicament the subject has received in the past fourteen qualified days. It should be noted that the number of qualified periods in a set of qualified periods may vary to accommodate subjects' different treatment plans and preferences. For example, a set of qualified periods may have 1 qualified period, 2 qualified periods, 5 qualified periods, 10 qualified periods, 14 qualified periods, or more than 14 qualified periods.

In some embodiments, a qualification criterion may be satisfied if the glucose level control system 1600 determines that the glucose level control system 1600 has been connected to (e.g., has been in communication with) an ambulatory medicament pump for at least a threshold period of time during the time period. The length of the threshold period of time may vary to accommodate different treatment plans and user preferences. For instance, the threshold period of time may be 1 to 24 hours, or longer than 24 hours. For example, the qualification criterion may be satisfied if the glucose level control system 1600 was connected to an ambulatory medicament pump for at least six hours during the time period. Alternatively, or in addition, the length of the threshold period of time may be a fraction of the length of the time period. For example, the qualification criterion may be satisfied if the glucose level control system 1600 was connected to an ambulatory medicament pump for at least 80% of the time period.

In some embodiments, a qualification criterion may be satisfied if the glucose level control system 1600 determines that the ambulatory medicament pump has been operatively connected to deliver therapy (e.g., administer medicament) to the subject for at least a threshold period of time during the time period. The length of the threshold period of time may vary to accommodate different treatment plans and user preferences. For instance, the threshold period of time may be 1 to 24 hours, or longer than 24 hours. For example, the qualification criterion may be satisfied if the ambulatory medicament pump was operatively connected to deliver therapy to the subject for at least eight hours during the time period. Alternatively, or in addition, the length of the threshold period of time may be a fraction of the length of the time period. For example, the qualification criterion may be satisfied if the ambulatory medicament pump was operatively connected to deliver therapy to the subject for at least 70% of the time period.

In some embodiments, a qualification criterion may be satisfied if the glucose level control system 1600 determines that the glucose level control system 1600 has been connected to a glucose level sensor (e.g., CGM sensor) for at least a threshold period of time during the time period. Alternatively, or in addition, the qualification criterion may include determining that the glucose level sensor has been operational and/or receiving glucose level data for at least the threshold percentage or amount of time within the time period. It should be understood that the amount of time may or may not be continuous. The length of the threshold period of time may vary to accommodate different treatment plans and user preferences. For instance, the threshold period of time may be 1 to 24 hours, or longer than 24 hours. For example, the qualification criterion may be satisfied if the glucose level control system 1600 has been connected to a glucose level sensor (e.g., CGM sensor) for at least ten hours during the time period. Alternatively, or in addition, the length of the threshold period of time may be a fraction of the length of the time period. For example, the qualification criterion may be satisfied if the glucose level control system 1600 has been connected to a glucose level sensor (e.g., CGM sensor) for at least 60% of the time period.

In some embodiments, a qualification criterion may be satisfied if the glucose level control system 1600 determines that the glucose level control system 1600 has received glucose level data that corresponds to at least a threshold period of time during the time period. The length of the threshold period of time may vary to accommodate different treatment plans and user preferences. For instance, the threshold period of time may be 1 to 24 hours, or longer than 24 hours. For example, the qualification criterion may be satisfied if the glucose level control system 1600 has received glucose level data that corresponds to at least a twelve-hour period during the time period. Alternatively, or in addition, the length of the threshold period of time may be a fraction of the length of the time period. For example, the qualification criterion may be satisfied if the glucose level control system 1600 has received glucose level data for at least 50% of the time period.

In some embodiments, a qualification criterion may be satisfied if the glucose level control system 1600 determines that the rate of change of the glucose level of the subject is below the threshold rate of change for at least a threshold period of time during the time period. The rate of change of the subject's glucose level may be indicated by or calculated from the subject's therapy data. In some embodiments, the glucose level control system 1600 may qualify a time period if the threshold rate of change is a glucose level increase of 60 mg/dL per hour and the subject's glucose level increases less than 60 mg/dL per hour for at least the threshold period of time. Similarly, the glucose level control system 1600 may qualify the time period if the threshold rate of change is a glucose level decrease of 60 mg/dL per hour and the subject's glucose level decreases less than 60 mg/dL per hour for at least the threshold period of time. It should be noted that the threshold rate of change for the qualification criteria may vary to accommodate different preferences and therapy treatment plans of the subject. For example, the threshold rate of change for the qualification criteria may be a glucose level increase and/or decrease of 30 mg/dL, 60 mg/dL, 90 mg/dL, 120 mg/dL, or greater than 120 mg/dL during a certain time period. The time period may vary to accommodate different preferences and therapy treatment plans. For example, the time period may be 30 minutes, 1 hour, 2 hours, 4 hours, 8 hours, or more than 8 hours. In some embodiments, the glucose level control system 1600 may have a different rate of change threshold for a glucose level increase than a glucose level decrease. For example, the threshold rate of change for the qualification criteria may be an increase of 100 mg/dL per hour and a decrease of 50 mg/dL per hour.

In some embodiments, the length of the threshold period of time may vary to accommodate different treatment plans and user preferences. For instance, the threshold period of time may be 1 to 24 hours, or longer than 24 hours. Alternatively, or in addition, the length of the threshold period of time may be a fraction of the length of the time period. For example, the qualification criterion may be satisfied if the threshold rate of change for the qualification criterion is a glucose level increase of 60 mg/dL per hour and the subject's glucose level increases less than 60 mg/dL per hour for at least 45% of the time period. In some embodiments, the length of the threshold period of time is the entire length of the time period. Thus, the qualification criterion may be satisfied if the threshold rate of change for the qualification criterion is a glucose level increase of 80 mg/dL per hour and the subject's glucose level increases less than 80 mg/dL per hour for the entire time period.

In some embodiments, the threshold periods of time for the different qualification criteria (e.g., glucose level control system 1600 has been connected to the ambulatory medicament pump for a threshold period, ambulatory medicament pump has been operatively connected to deliver therapy for a threshold period, glucose level control system 1600 has been connected to glucose level sensor for a threshold period, etc.) may have different lengths. For example, a first threshold period of time may be six hours and a second threshold period of time may be eight hours. Alternatively, two or more of the aforementioned threshold periods of time may have the same length. For example, two or more of the aforementioned threshold periods of time may be 12 hours, 24 hours, or 36 hours, etc.

In some embodiments, a qualification criterion may be satisfied if the glucose level control system 1600 determines that the therapy data of the time period differs from one or more instances of qualified therapy data (e.g., therapy data that corresponds to a qualified time period) by no more than a threshold difference. Alternatively, or in addition, the glucose level control system 1600 may qualify a time period if it determines that the therapy data of the time period differs from the average of one or more instances of qualified therapy data by no more than a threshold difference.

In some embodiments, the glucose level control system 1600 may determine the difference between an aspect of the subject's glucose level data from a time period and a corresponding aspect of the subject's glucose level data from one or more qualified time periods. The glucose level control system may qualify the time period if the difference is below a threshold difference. The aspect of the subject's glucose level may include the maximum, minimum, average, and/or standard deviation of the glucose level during the time period. For instance, the glucose level control system 1600 may compare the subject's maximum glucose level during the time period and a qualified time period to determine if the difference of the two maximum glucose levels is less than the threshold difference (e.g., ±30 mg/dL). If the difference between the maximum glucose levels is less than the threshold difference, the glucose level control system 1600 may qualify the time period as a qualified time period. In contrast, if the difference between the maximum glucose levels is greater than the threshold difference, the glucose level control system 1600 may not qualify the time period as a qualified time period.

In some embodiments, the glucose level control system 1600 may compare an aspect of the subject's glucose level data (e.g., maximum, minimum, etc.) corresponding to a time period to an average of the same aspect from a set of qualified time periods. For instance, the glucose level control system 1600 may compare the subject's minimum glucose level during the time period and the average minimum glucose level of a set of qualified days to determine if the difference between the minimum glucose levels is less than the threshold difference (e.g., ±50 mg/dL). It should be noted that the threshold difference may vary to accommodate users' different needs and preferences. For instance, the threshold difference for a comparison based on the subject's glucose level data may be anywhere between ±0 mg/dL to ±120 mg/dL, or more than ±120 mg/dL. The threshold difference may be different for each qualification criterion. For example, the threshold difference for the minimum glucose level may be ±50 mg/dL while the threshold difference for the standard deviation of the subject's glucose level may be ±20 mg/dL.

In some embodiments, the glucose level control system 1600 may determine if a time period satisfies a qualification criterion by comparing one or more aspects of the medicament delivery data corresponding to the time period and one or more qualified time periods. For example, the glucose level control system 1600 may compare the total amount of insulin the subject received during the time period and a qualified time period to determine if the difference between the total amount of insulin received is less than the threshold difference (e.g., ±75 units of insulin). Alternatively, or in addition, the glucose level control system 1600 may compare the total number of administered medicament doses during a time period and a qualified time period to determine if the difference between the total number of administered medicament doses is less than the threshold difference (e.g., ±3 administered medicament doses per day).

In some embodiments, the glucose level control system 1600 may qualify a time period if the glucose level control system 1600 determines that the glucose level of the subject indicated by the therapy data is within a setpoint range for at least a threshold percentage of the time period. The threshold percentage may be any percentage (e.g., 0%-100%) to accommodate different needs and preferences. For example, a qualification criterion may be satisfied if the glucose level of the subject stays within the setpoint range for 75% of the time period. In some embodiments, the threshold percentage may be a threshold period of time. For example, the qualification criterion may be satisfied if the glucose level of the subject stays within the setpoint range for at least six hours during the time period.

In some embodiments, the glucose level control system 1600 may qualify a time period based at least in part on one or more user-defined qualification criteria. In some embodiments, a user may enter the user-defined qualification criteria via a user interface of the glucose level control system 1600. Alternatively, a user may enter the user-defined qualification criteria via a user interface on another electronic device (e.g., via an application on a smartphone). User-defined qualification criteria may include any of the qualification criteria described above. Alternatively, or in addition, user-defined qualification criteria may include a determination that one or more administered medicament doses during a time period were of a certain type, amount, concentration, or brand of medicament. For example, a user-defined criteria may be satisfied if the administered medicament doses corresponding to the time period had a concentration of U-300 or below. Similarly, a user-defined criteria may be satisfied if the administered medicament doses corresponding to the time period were doses of long-acting insulin. It should be noted that user-define qualification criteria may include any criteria corresponding to the therapy data, glucose level data, medicament data, and/or user-inputted data corresponding to the time period.

In some embodiments, the glucose level control system 1600 may qualify one or more user-selected time periods. For example, a user may, via a user interface, review a log of past time periods and select time periods that most closely represent the subject's typical schedule (e.g., time periods where the subject's eating habits, sleep schedule, and/or workout habits were not out of the ordinary for the subject). Alternatively, or in addition, a user may manually remove qualified periods from a qualified set. For example, if the glucose level control system 1600 qualified a time period that does not represent the subject's normal schedule and habits, the user may remove the time period from the qualified set. For instance, a user may remove a qualified time period if the subject received a medical procedure during the time period. Similarly, a user may remove a qualified time period if the subject's physical activity during the time period was out of the ordinary (e.g., the subject participated in a marathon during the time period). It should be noted that a user may add or remove a time period from a qualified set for any reason.

In some embodiments, the glucose level control system 1600 determines a recommended medicament dose that is a fraction of the total daily dose of medicament It should be noted that the size of the fraction for the recommended medicament dose may vary to accommodate different subjects' needs and preferences. For example, a recommended medicament dose may be 20%, 40%, 60%, 80%, or more than 80% of the total daily dose of medicament. In some embodiments, the glucose level control system 1600 determines the size of the fraction of the total daily dose for the recommended medicament dose based at least in part on the qualitative meal announcement. For example, the glucose level control system 1600 may determine a recommended medicament dose that is 15% of the total daily dose of insulin when a breakfast meal announcement is received, a recommended medicament dose that is 30% of the total daily dose of insulin when a lunch meal announcement is received, and a recommended medicament dose that is 40% of the total daily dose of insulin when a dinner meal announcement is received. Similarly, the glucose level control system 1600 may determine a recommended medicament dose that is 5% of the total daily dose of insulin when a snack meal announcement is received, a recommended medicament dose that is 10% of the total daily dose of insulin when a small meal announcement is received, a recommended medicament dose that is 20% of the total daily dose of insulin when a medium meal announcement is received, and a recommended medicament dose that is 35% of the total daily dose of insulin when a large meal announcement is received.

In some embodiments, the glucose level control system compares a subject's current daily dose of insulin to the subject's total daily dose of insulin to determine a recommended medicament dose. For example, the glucose level control system 1600 may determine a larger recommended medicament dose if the difference between the subject's current daily dose of insulin and the subject's total daily dose of insulin is large (e.g., the difference is greater than 50 units of insulin). Similarly, the glucose level control system 1600 may determine a smaller recommended medicament dose if the difference between the subject's current daily dose of insulin and the subject's total daily dose of insulin is small (e.g., the difference is less than 50 units of insulin). Alternatively, or in addition, the glucose level control system 1600 may determine a recommended medicament dose of zero units of insulin if the subject's current daily dose of insulin is larger than the subject's total daily dose of insulin. In some embodiments, the block 2104 may include one or more of the embodiments previously described with respect to the block 1806, the block 1906, and the block 2006.

At block 2106, the glucose level control system 1600 may receive an indication that the user intends to deliver a medicament dose to the subject. In some embodiments, the user indicates the medicament delivery device that may be used to deliver the medicament dose. For example, the user may indicate that the medicament delivery device is a non-automated medicament delivery device (e.g., the smart pen 1602 or a non-automated insulin pump). The glucose level control system 1600 may receive the indication that the user intends to deliver a medicament dose via a dosing control interface. Alternatively, or in addition, the user may indicate information about the intended medicament dose via the dosing control interface. For example, the user may specify that the intended medicament dose is the recommended medicament dose (e.g., by entering a medicament dose amount) determined at block 2104. In some embodiments, the user may input medicament delivery data (e.g., the type, size, and/or concentration of the medicament dose) that corresponds with the intended medicament dose. In some embodiments, the block 2106 may include one or more of the embodiments previously described with respect to the block 1904 and the block 1908.

At block 2108, the glucose level control system 1600 outputs an indication of the recommended medicament dose in response to receiving the indication that the user intends to deliver a dose of medicament. For example, the glucose level control system 1600 can transmit the indication of the recommended medicament dose to a medicament delivery device, such as the smart pen 1602. The indication of the recommended medicament dose enables the smart pen 1602 to display the recommended medicament dose. The smart pen 1602 can display the indication of the recommended dose of medicament on the display 1710 of the smart pen 1602. Alternatively, or in addition, the smart pen 1602 may be automatically configured based on the recommended medicament dose. In some embodiments, the block 2108 may include one or more of the embodiments previously described with respect to the block 1808 and the block 1910.

### User Interface

FIG. 22 illustrates an example user interface 2200 for medicament dose recommendations in accordance with certain embodiments. One or more of the embodiments of the glucose level control system 1600 disclosed herein may include the user interface 2200 and may receive data from the user interface 2200. The glucose level control system 1600, the smart pen 1602, and/or an electronic device in communication with the glucose level control system 1600 may generate or display the user interface 2200. In some embodiments, the user interface 2200 includes four buttons (e.g., buttons 2215 and indicator buttons 2220). The user interface 2200 represents just one non-limiting example of user interfaces that may be generated. It is possible for other user interfaces to be generated that include more or less data and/or user interaction options such as a touchscreen, buttons, or UI elements. For example, the tables 2205 and 2210 may be displayed on two separate user interface screens that may be separately generated and/or accessed.

The dose history of the smart pen 1602 may be displayed in the table 2205 and may indicate medicament doses that were administered to the subject via the smart pen 1602. Alternatively, or in addition, the table 2205 may display any medicament delivery data corresponding to the smart pen 1602, an ambulatory medicament device, or any medicament delivery device. For example, the table 2205 may include the date and time that one or more medicament doses were administered. Alternatively, or in addition, the table 2205 may include the medicament type (e.g., long-acting insulin, fast-acting insulin, etc.) and medicament size. In some embodiments, the table 2205 may include the medicament concentration, dose type (e.g., meal dose, basal rate dose, or correction dose), medicament brand (e.g., Novolog, Humalog, etc.), or any other information corresponding to the medicament dose. In some embodiments, a table 2205 may display the dose history corresponding to a user-selected time period (e.g., an hour, day, week, etc.). A user can select a time period using the buttons (e.g., buttons 2215 or indicator buttons 2220) or UI elements of the user interface.

In some embodiments, the user interface 2200 may display one or more recommended medicament doses. The recommended medicament doses may be displayed in the table 2210. The table 2210 may display any medicament delivery data corresponding to the recommended medicament dose. For example, the table 2210 may include the medicament type (e.g., long-acting insulin, fast-acting insulin, etc.) and the medicament size of the recommended medicament dose. Alternatively, or in addition, the table 2210 may include the date and time that the recommended medicament dose was generated by the glucose level control system 1600. The table 2210 may also, or alternatively, include a recommended date and time to administer the recommended medicament dose. In some embodiments, the table 2210 may include the medicament concentration, dose type (e.g., meal dose, basal rate dose, and/or correction dose), medicament brand, or any other information corresponding to the recommended medicament dose. Alternatively, or in addition, the table 2210 may indicate a recommended delivery method (e.g., the smart pen 1602, an ambulatory medicament device, or any other delivery device). In some embodiments, a table 2210 may display a log of past recommended medicament doses and any medicament delivery data corresponding to the past recommended medicament doses.

As described above, the user interface 2200 may include buttons 2215, indicator buttons 2220, and/or UI elements (e.g., a touchscreen, on-screen options, etc.). Such buttons 2215, indicator buttons 2220, and UI elements may allow the user to interact with the interface 2200. For example, buttons 2215 may allow the user to indicate that the user acknowledges the recommended medicament dose and/or has or will administer the recommended medicament dose. The buttons 2215 may allow the user to indicate that the user has or will administer a medicament dose that differs from the recommended medicament dose. The user may also, or alternatively, input (e.g., via the indicator buttons 2220 or a number pad) the different medicament dose. Further, the buttons 2215, indicator button 2220, and/or UI elements may allow the user to indicate that the user has decided to dismiss or ignore the medicament dose recommendation. Further, UI elements may include a confirmation interface asking the user to confirm the user's selection. The user's indication via the buttons 2215, indicator button 2220, and/or UI elements may be recorded in a log and/or transmitted to another electronic device that may be accessible by another user (e.g., a clinician or other healthcare provider, a parent or guardian, or other authorized user). In some embodiments, the user interface 2200 may display information about the glucose level control system 1600, such as the amount of battery life remaining. The user interface may also, or alternatively, display a subject's and/or user's name and the local time and date.

### Terminology

It is to be understood that not necessarily all objects or advantages may be achieved in accordance with any particular embodiment described herein. Thus, for example, those skilled in the art will recognize that certain embodiments may be configured to operate in a manner that achieves or optimizes one advantage or group of advantages as taught herein without necessarily achieving other objects or advantages as may be taught or suggested herein. Further, it should be understood that certain embodiments may be combinable or combined with certain other embodiments described herein.

All of the processes described herein may be embodied in, and fully automated via, software code modules executed by a computing system that includes one or more computers or processors. The code modules may be stored in any type of non-transitory computer-readable medium or other computer storage device. Some or all the methods may be embodied in specialized computer hardware. Further, the computing system may include, be implemented as part of, or communicate with a glucose level control system, an automated blood glucose system, an ambulatory medicament system, or an ambulatory medical device.

Many other variations than those described herein will be apparent from this disclosure. For example, depending on the embodiment, certain acts, events, or functions of any of the algorithms described herein can be performed in a different sequence, can be added, merged, or left out altogether (for example, not all described acts or events are necessary for the practice of the algorithms). Moreover, in certain embodiments, acts or events can be performed concurrently, for example, through multi-threaded processing, interrupt processing, or multiple processors or processor cores or on other parallel architectures, rather than sequentially. In addition, different tasks or processes can be performed by different machines and/or computing systems that can function together.

The various illustrative logical blocks and modules described in connection with the embodiments disclosed herein can be implemented or performed by a machine, such as a processing unit or processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor can be a microprocessor, but in the alternative, the processor can be a controller, microcontroller, or state machine, combinations of the same, or the like. A processor can include electrical circuitry configured to process computer-executable instructions. In another embodiment, a processor includes an FPGA or other programmable device that performs logic operations without processing computer-executable instructions. A processor can also be implemented as a combination of computing devices, for example, a combination of a DSP and a microprocessor, a plurality of microprocessors, one or more microprocessors in conjunction with a DSP core, or any other such configuration. Although described herein primarily with respect to digital technology, a processor may also include primarily analog components. A computing environment can include any type of computer system, including, but not limited to, a computer system based on a microprocessor, a mainframe computer, a digital signal processor, a portable computing device, a device controller, or a computational engine within an appliance, to name a few.

Conditional language such as, among others, “can,” “could,” “might” or “may,” unless specifically stated otherwise, are otherwise understood within the context as used in general to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment.

Disjunctive language such as the phrase “at least one of X, Y, or Z,” unless specifically stated otherwise, is otherwise understood with the context as used in general to present that an item, term, etc., may be either X, Y, or Z, or any combination thereof (for example, X, Y, and/or Z). Thus, such disjunctive language is not generally intended to, and should not, imply that certain embodiments require at least one of X, at least one of Y, or at least one of Z to each be present.

Any process descriptions, elements or blocks in the flow diagrams described herein and/or depicted in the attached figures should be understood as potentially representing modules, segments, or portions of code which include one or more executable instructions for implementing specific logical functions or elements in the process. Alternate implementations are included within the scope of the embodiments described herein in which elements or functions may be deleted, executed out of order from that shown, or discussed, including substantially concurrently or in reverse order, depending on the functionality involved as would be understood by those skilled in the art.

Unless otherwise explicitly stated, articles such as “a” or “an” should generally be interpreted to include one or more described items. Accordingly, phrases such as “a device configured to” are intended to include one or more recited devices. Such one or more recited devices can also be collectively configured to carry out the stated recitations. For example, “a processor configured to carry out recitations A, B and C” can include a first processor configured to carry out recitation A working in conjunction with a second processor configured to carry out recitations B and C.

It should be emphasized that many variations and modifications may be made to the above-described embodiments, the elements of which are to be understood as being among other acceptable examples. All such modifications and variations are intended to be included herein within the scope of this disclosure.

What is claimed is:

1. A glucose level control system configured to generate glucose control therapy recommendations for controlling a glucose level of a subject, the glucose level control system comprising:
a memory configured to store specific computer-executable instructions; and
a hardware processor in communication with the memory and configured to execute the specific computer-executable instructions to at least:
receive glucose level data associated with the glucose level of the subject;
receive, via user interaction with a dose setup interface, a user-specified medicament-related input related to an amount of insulin to be provided to the subject;
determine, using a control algorithm, a recommended dose of medicament based at least in part on the user-specified medicament-related input and the glucose level data, wherein the control algorithm is configured to control glucose level in the subject based on one or more control parameters of the control algorithm, and wherein at least one control parameter is set based at least in part on the glucose level data; and
output an indication of the recommended dose of medicament.

2. The glucose level control system of claim 1, wherein the hardware processor is further configured to execute the specific computer-executable instructions to at least:
receive second glucose level data associated with the glucose level of the subject;
receive, via second user interaction with the dose setup interface, a second user-specified medicament-related input that specifies a size of a second amount of insulin to be provided to the subject; and
determine a second recommended dose of medicament based on the second user-specified medicament-related input and the second glucose level data.

3. The glucose level control system of claim 2, wherein the hardware processor is further configured to execute the specific computer-executable instructions to at least:
determine that the second recommended dose of medicament matches the second user-specified medicament-related input within a threshold degree; and
output an indication of the second recommended dose of medicament.

4. The glucose level control system of claim 2, wherein the hardware processor is further configured to execute the specific computer-executable instructions to at least:
determine that the second recommended dose of medicament matches the second user-specified medicament-related input within a threshold degree; and
output an indication that the second user-specified medicament-related input satisfies an approval condition.

5. The glucose level control system of claim 2, wherein the hardware processor is further configured to execute the specific computer-executable instructions to at least:
determine that the second recommended dose of medicament does not match the second user-specified medicament-related input within a threshold degree; and
output an indication of a modification to the second user-specified medicament-related input based at least in part on a difference between the second recommended dose of medicament and the second user-specified medicament-related input.

6. The glucose level control system of claim 1, wherein at least one control parameter is set based at least in part on the user-specified medicament-related input.

7. The glucose level control system of claim 1, wherein the glucose level data is received from a glucose sensor operatively coupled to the subject.

8. The glucose level control system of claim 1, wherein the glucose level data comprises one or more isolated glucose measurements.

9. The glucose level control system of claim 1, wherein the user-specified medicament-related input comprises a fast-acting insulin dose size.

10. The glucose level control system of claim 1, wherein the user-specified medicament-related input comprises a long-acting insulin dose size.

11. The glucose level control system of claim 1, wherein the user-specified medicament-related input comprises a user-specified volume of insulin.

12. The glucose level control system of claim 1, wherein the user-specified medicament-related input comprises a quantitative indication of carbohydrates, and wherein the recommended dose of medicament is based at least in part on the quantitative indication of the carbohydrates.

13. The glucose level control system of claim 1, wherein the user-specified medicament-related input comprises a user-specified quantitative characterization of an amount of food-intake and a macronutrient content of the food-intake, and wherein the recommended dose of medicament is based at least in part on the user-specified quantitative characterization of the amount of food-intake and the macronutrient content of the food-intake.

14. The glucose level control system of claim 1, wherein the control algorithm is further configured to account for a first accumulation of recommended doses of medicament for the subject over time.

15. The glucose level control system of claim 14, wherein the control algorithm accounts for the first accumulation of recommended doses of medicament by reducing the recommended dose of medicament based at least in part on the first accumulation of recommended doses of medicament.

16. The glucose level control system of claim 15, wherein outputting the indication of the recommended dose of medicament comprises outputting the reduced recommended dose of medicament.

17. The glucose level control system of claim 15, wherein the reduced recommended dose of medicament is added to the first accumulation of recommended doses of medicament to obtain a second accumulation of recommended doses of medicament.

18. The glucose level control system of claim 17, wherein said outputting the indication of the recommended dose of medicament comprises outputting an indication of the second accumulation of recommended doses of medicament.

19. The glucose level control system of claim 14, wherein the control algorithm accounts for the accumulation of recommended doses of medicament by adding the recommended dose of medicament to the first accumulation of recommended doses of medicament to obtain a second accumulation of recommended doses of medicament.

20. The glucose level control system of claim 19, wherein said outputting the indication of the recommended dose of medicament comprises outputting an indication of the second accumulation of recommended doses of medicament.

21. The glucose level control system of claim 14, wherein the first accumulation of recommended doses of medicament comprises an accumulation of basal insulin doses.

22. The glucose level control system of claim 21, wherein the first accumulation of recommended doses of medicament further comprises at least one correction dose of medicament.

23. The glucose level control system of claim 1, wherein the control algorithm is further configured to adapt at least one of the one or more control parameters in response to determined glycemic control associated with medicament provided during a prior therapy period.

24. The glucose level control system of claim 23, wherein the determined glycemic control is determined based at least in part on the glucose level data, prior glucose level data associated with the prior therapy period, or both the glucose level data and the prior glucose level data.

25. The glucose level control system of claim 24, wherein one or more of the glucose level data or the prior glucose level data is received from a glucose sensor operatively coupled to the subject.

26. The glucose level control system of claim 24, wherein one or more of the glucose level data or the prior glucose level data is determined from one or more isolated glucose measurements.

27. The glucose level control system of claim 1, wherein the hardware processor is further configured to output the indication of the recommended dose of medicament on a display of the glucose level control system.

28. The glucose level control system of claim 1, wherein the hardware processor is further configured to output the indication of the recommended dose of medicament by at least causing the indication of the recommended dose of medicament to be transmitted to an electronic device in communication with the glucose level control system.

29. The glucose level control system of claim 28, wherein the electronic device comprises a smart pen.

30. The glucose level control system of claim 29, wherein the electronic device comprises a smart insulin pen.
